SEQUENCE LISTING (98 sequences)

SEQ ID NO: 1 - RNA, 24 nt, Artificial Sequence
  miR-29a mimic second/ sense/ passenger strand
  modified_base (1)..(1): 2′-O-methyl-uridine
  modified_base (2)..(2): 2′-O-methyl-adenosine
uaaccgauuucagauggugcuauu

SEQ ID NO: 2 - RNA, 22 nt, Artificial Sequence
  miR-29a mimic second/ sense/ passenger strand
  modified_base (1)..(1): 2′-O-methyl-uridine
  modified_base (2)..(2): 2′-O-methyl-adenosine
  modified_base (4)..(4): 2′-O-methyl-cytidine
  modified_base (5)..(5): 2′-O-methyl-cytidine
  modified_base (7)..(7): 2′-O-methyl-uridine
  modified_base (8)..(8): 2′-O-methyl-uridine
  modified_base (9)..(9): 2′-O-methyl-uridine
  modified_base (11)..(11): 2′-O-methyl-cytidine
  modified_base (15)..(15): 2′-O-methyl-uridine
  modified_base (18)..(18): 2′-O-methyl-uridine
  modified_base (19)..(19): 2′-O-methyl-cytidine
  modified_base (20)..(20): 2′-O-methyl-cytidine
  modified_base (21)..(21): 2′-O-methyl-uridine
uaaccguuuacagaugguccua

SEQ ID NO: 3 - RNA, 22 nt, Artificial Sequence
  miR-29a mimic second/ sense/ passenger strand
  modified_base (1)..(1): 2′-O-methyl-uridine
  modified_base (2)..(2): 2′-O-methyl-adenosine
  modified_base (4)..(4): 2′-O-methyl-cytidine
  modified_base (5)..(5): 2′-O-methyl-cytidine
  modified_base (7)..(7): 2′-O-methyl-uridine
  modified_base (8)..(8): 2′-O-methyl-uridine
  modified_base (9)..(9): 2′-O-methyl-uridine
  modified_base (11)..(11): 2′-O-methyl-cytidine
  modified_base (15)..(15): 2′-O-methyl-uridine
  modified_base (18)..(18): 2′-O-methyl-uridine
  modified_base (19)..(19): 2′-O-methyl-cytidine
  modified_base (20)..(20): 2′-O-methyl-cytidine
  modified_base (21)..(21): 2′-O-methyl-uridine
  misc_feature (22)..(22): Cholesterol conjugate with a 6 carbon linker
uaaccguuuacagaugguccua

SEQ ID NO: 4 - RNA, 24 nt, Artificial Sequence
  miR-29a mimic second/ sense/ passenger strand
  modified_base (1)..(1): 2′-O-methyl-uridine
  modified_base (2)..(2): 2′-O-methyl-adenosine
  misc_feature (22)..(24): joined through phosphorothioate bonds
  misc_feature (24)..(24): Cholesterol conjugate with a 6 carbon linker linked via phosphorothioate bond
uaaccgauuucagauggugcuauu

SEQ ID NO: 5 - RNA, 22 nt, Artificial Sequence
  miR-29a mimic second/ sense/ passenger strand
  modified_base (1)..(1): 2′-O-methyl-uridine
  modified_base (2)..(2): 2′-O-methyl-adenosine
uaaccgauuucagauggugcua

SEQ ID NO: 6 - RNA, 24 nt, Artificial Sequence
  miR-29a mimic first/ antisense/ guide strand
  misc_feature (1)..(1): 5′-terminal monophosphate
uagcaccaucugaaaucgguuauu

SEQ ID NO: 7 - RNA, 24 nt, Artificial Sequence
  miR-29a mimic first/ antisense/ guide strand
  misc_feature (1)..(1): 5′-terminal monophosphate
  modified_base (1)..(1): 2′-fluoro-uridine
  modified_base (4)..(4): 2′-fluoro-cytidine
  modified_base (6)..(6): 2′-fluoro-cytidine
  modified_base (7)..(7): 2′-fluoro-cytidine
  modified_base (9)..(9): 2′-fluoro-uridine
  modified_base (10)..(10): 2′-fluoro-cytidine
  modified_base (11)..(11): 2′-fluoro-uridine
  modified_base (16)..(16): 2′-fluoro-uridine
  modified_base (17)..(17): 2′-fluoro-cytidine
  modified_base (20)..(20): 2′-fluoro-uridine
  modified_base (21)..(21): 2′-fluoro-uridine
  misc_feature (22)..(24): joined through phosphorothioate bonds
uagcaccaucugaaaucgguuauu

SEQ ID NO: 8 - RNA, 24 nt, Artificial Sequence
  miR-29a mimic first/ antisense/ guide strand
  modified_base (1)..(1): 2′-fluoro-uridine
  modified_base (4)..(4): 2′-fluoro-cytidine
  modified_base (6)..(6): 2′-fluoro-cytidine
  modified_base (7)..(7): 2′-fluoro-cytidine
  modified_base (9)..(9): 2′-fluoro-uridine
  modified_base (10)..(10): 2′-fluoro-cytidine
  modified_base (11)..(11): 2′-fluoro-uridine
  modified_base (16)..(16): 2′-fluoro-uridine
  modified_base (17)..(17): 2′-fluoro-cytidine
  modified_base (20)..(20): 2′-fluoro-uridine
  modified_base (21)..(21): 2′-fluoro-uridine
  misc_feature (22)..(24): joined through phosphorothioate bonds
uagcaccaucugaaaucgguuauu

SEQ ID NO: 9 - RNA, 24 nt, Artificial Sequence
  miR-29a mimic first/ antisense/ guide strand
uagcaccaucugaaaucgguuauu

SEQ ID NO: 10 - RNA, 25 nt, Artificial Sequence
  miR-29b mimic second/ sense/ passenger strand
  modified_base (1)..(1): 2′-O-methyl-adenosine
  modified_base (2)..(2): 2′-O-methyl-adenosine
aacacugauuucaaauggugcuauu

SEQ ID NO: 11 - RNA, 23 nt, Artificial Sequence
  miR-29b mimic second/ sense/ passenger strand
  modified_base (1)..(1): 2′-O-methyl-adenosine
  modified_base (2)..(2): 2′-O-methyl-adenosine
  modified_base (3)..(3): 2′-O-methyl-cytidine
  modified_base (5)..(5): 2′-O-methyl-cytidine
  modified_base (6)..(6): 2′-O-methyl-uridine
  modified_base (9)..(9): 2′-O-methyl-uridine
  modified_base (10)..(10): 2′-O-methyl-uridine
  modified_base (11)..(11): 2′-O-methyl-uridine
  modified_base (12)..(12): 2′-O-methyl-cytidine
  modified_base (16)..(16): 2′-O-methyl-uridine
  modified_base (19)..(19): 2′-O-methyl-uridine
  modified_base (21)..(21): 2′-O-methyl-cytidine
  modified_base (22)..(22): 2′-O-methyl-uridine
  misc_feature (23)..(23): Cholesterol conjugate with a 6 carbon linker
aacacugauuucaaauggugcua

SEQ ID NO: 12 - RNA, 25 nt, Artificial Sequence
  miR-29b mimic second/ sense/ passenger strand
  modified_base (1)..(1): 2′-O-methyl-adenosine
  modified_base (2)..(2): 2′-O-methyl-adenosine
  misc_feature (23)..(25): joined through phosphorothioate bonds
  misc_feature (25)..(25): Cholesterol conjugate with a 6 carbon linker via phosphorothioate bond
aacacugauuucaaauggugcuauu

SEQ ID NO: 13 - RNA, 23 nt, Artificial Sequence
  miR-29b mimic second/ sense/ passenger strand
  modified_base (1)..(1): 2′-O-methyl-adenosine
  modified_base (2)..(2): 2′-O-methyl-adenosine
  modified_base (3)..(3): 2′-O-methyl-cytidine
  modified_base (5)..(5): 2′-O-methyl-cytidine
  modified_base (6)..(6): 2′-O-methyl-uridine
  modified_base (8)..(8): 2′-O-methyl-uridine
  modified_base (9)..(9): 2′-O-methyl-uridine
  modified_base (10)..(10): 2′-O-methyl-uridine
  modified_base (12)..(12): 2′-O-methyl-cytidine
  modified_base (16)..(16): 2′-O-methyl-uridine
  modified_base (19)..(19): 2′-O-methyl-uridine
  modified_base (20)..(20): 2′-O-methyl-cytidine
  modified_base (21)..(21): 2′-O-methyl-cytidine
  modified_base (22)..(22): 2′-O-methyl-uridine
aacacuguuuacgggugguccua

SEQ ID NO: 14 - RNA, 23 nt, Artificial Sequence
  miR-29b mimic second/ sense/ passenger strand
  modified_base (1)..(1): 2′-O-methyl-adenosine
  modified_base (2)..(2): 2′-O-methyl-adenosine
  modified_base (3)..(3): 2′-O-methyl-cytidine
  modified_base (5)..(5): 2′-O-methyl-cytidine
  modified_base (6)..(6): 2′-O-methyl-uridine
  modified_base (8)..(8): 2′-O-methyl-uridine
  modified_base (9)..(9): 2′-O-methyl-uridine
  modified_base (10)..(10): 2′-O-methyl-uridine
  modified_base (12)..(12): 2′-O-methyl-cytidine
  modified_base (16)..(16): 2′-O-methyl-uridine
  modified_base (19)..(19): 2′-O-methyl-uridine
  modified_base (20)..(20): 2′-O-methyl-cytidine
  modified_base (21)..(21): 2′-O-methyl-cytidine
  modified_base (22)..(22): 2′-O-methyl-uridine
  misc_feature (23)..(23): Cholesterol conjugate with a 6 carbon linker
aacacuguuuacgggugguccua

SEQ ID NO: 15 - RNA, 23 nt, Artificial Sequence
  miR-29b mimic second/ sense/ passenger strand
  modified_base (1)..(1): 2′-O-methyl-adenosine
  modified_base (2)..(2): 2′-O-methyl-adenosine
  modified_base (3)..(3): 2′-O-methyl-cytidine
  modified_base (5)..(5): 2′-O-methyl-cytidine
  modified_base (6)..(6): 2′-O-methyl-uridine
  modified_base (8)..(8): 2′-O-methyl-uridine
  modified_base (9)..(9): 2′-O-methyl-uridine
  modified_base (10)..(10): 2′-O-methyl-uridine
  modified_base (12)..(12): 2′-O-methyl-cytidine
  modified_base (16)..(16): 2′-O-methyl-uridine
  modified_base (19)..(19): 2′-O-methyl-uridine
  modified_base (20)..(20): 2′-O-methyl-cytidine
  modified_base (21)..(21): 2′-O-methyl-cytidine
  modified_base (22)..(22): 2′-O-methyl-uridine
  misc_feature (23)..(23): Cholesterol conjugate with a 6 carbon linker
aacacuguuuacaaaugguccua

SEQ ID NO: 16 - RNA, 25 nt, Artificial Sequence
  miR-29b mimic second/ sense/ passenger strand
  modified_base (1)..(1): 2′-O-methyl-adenosine
  modified_base (2)..(2): 2′-O-methyl-adenosine
  modified_base (3)..(3): 2′-O-methyl-cytidine
  modified_base (5)..(5): 2′-O-methyl-cytidine
  modified_base (6)..(6): 2′-O-methyl-uridine
  modified_base (8)..(8): 2′-O-methyl-uridine
  modified_base (9)..(9): 2′-O-methyl-uridine
  modified_base (10)..(10): 2′-O-methyl-uridine
  modified_base (12)..(12): 2′-O-methyl-cytidine
  modified_base (16)..(16): 2′-O-methyl-uridine
  modified_base (19)..(19): 2′-O-methyl-uridine
  modified_base (20)..(20): 2′-O-methyl-cytidine
  modified_base (21)..(21): 2′-O-methyl-cytidine
  modified_base (22)..(22): 2′-O-methyl-uridine
  modified_base (24)..(24): deoxythymidine
  modified_base (25)..(25): deoxythymidine
  misc_feature (25)..(25): Cholesterol conjugate with a 6 carbon linker
aacacuguuuacaaaugguccuann

SEQ ID NO: 17 - RNA, 25 nt, Artificial Sequence
  miR-29b mimic second/ sense/ passenger strand
  misc_feature (1)..(1): Cholesterol conjugate with a 6 carbon linker
  modified_base (1)..(1): deoxythymidine
  modified_base (2)..(2): deoxythymidine
  modified_base (3)..(3): 2′-O-methyl-adenosine
  modified_base (4)..(4): 2′-O-methyl-adenosine
  modified_base (5)..(5): 2′-O-methyl-cytidine
  modified_base (7)..(7): 2′-O-methyl-cytidine
  modified_base (8)..(8): 2′-O-methyl-uridine
  modified_base (10)..(10): 2′-O-methyl-uridine
  modified_base (11)..(11): 2′-O-methyl-uridine
  modified_base (12)..(12): 2′-O-methyl-uridine
  modified_base (14)..(14): 2′-O-methyl-cytidine
  modified_base (18)..(18): 2′-O-methyl-uridine
  modified_base (21)..(21): 2′-O-methyl-uridine
  modified_base (22)..(22): 2′-O-methyl-cytidine
  modified_base (23)..(23): 2′-O-methyl-cytidine
  modified_base (24)..(24): 2′-O-methyl-uridine
nnaacacuguuuacaaaugguccua

SEQ ID NO: 18 - RNA, 23 nt, Artificial Sequence
  miR-29b mimic second/ sense/ passenger strand
  modified_base (1)..(1): 2′-O-methyl-adenosine
  modified_base (2)..(2): 2′-O-methyl-adenosine
  modified_base (3)..(3): 2′-O-methyl-cytidine
  modified_base (5)..(5): 2′-O-methyl-cytidine
  modified_base (6)..(6): 2′-O-methyl-uridine
  modified_base (8)..(8): 2′-O-methyl-uridine
  modified_base (9)..(9): 2′-O-methyl-uridine
  modified_base (10)..(10): 2′-O-methyl-uridine
  modified_base (12)..(12): 2′-O-methyl-cytidine
  modified_base (16)..(16): 2′-O-methyl-uridine
  modified_base (19)..(19): 2′-O-methyl-uridine
  modified_base (20)..(20): 2′-O-methyl-cytidine
  modified_base (21)..(21): 2′-O-methyl-cytidine
  modified_base (22)..(22): 2′-O-methyl-uridine
  misc_feature (23)..(23): Cholesterol conjugate with a 9 carbon linker
aacacuguuuacaaaugguccua

SEQ ID NO: 19 - RNA, 23 nt, Artificial Sequence
  miR-29b mimic second/ sense/ passenger strand
  modified_base (2)..(2): 2′-O-methyl-adenosine
  modified_base (4)..(4): 2′-O-methyl-adenosine
  modified_base (6)..(6): 2′-O-methyl-uridine
  modified_base (8)..(8): 2′-O-methyl-adenosine
  modified_base (10)..(10): 2′-O-methyl-uridine
  modified_base (12)..(12): 2′-O-methyl-cytidine
  modified_base (14)..(14): 2′-O-methyl-adenosine
  modified_base (16)..(16): 2′-O-methyl-uridine
  modified_base (18)..(18): 2′-O-methyl-guanosine
  modified_base (20)..(20): 2′-O-methyl-guanosine
  modified_base (22)..(22): 2′-O-methyl-uridine
  misc_feature (23)..(23): Cholesterol conjugate with a 6 carbon linker
aacacugauuucaaauggugcua

SEQ ID NO: 20 - RNA, 25 nt, Artificial Sequence
  miR-29b mimic second/ sense/ passenger strand
  modified_base (2)..(2): 2′-O-methyl-adenosine
  modified_base (4)..(4): 2′-O-methyl-adenosine
  modified_base (6)..(6): 2′-O-methyl-uridine
  modified_base (8)..(8): 2′-O-methyl-adenosine
  modified_base (10)..(10): 2′-O-methyl-uridine
  modified_base (12)..(12): 2′-O-methyl-cytidine
  modified_base (14)..(14): 2′-O-methyl-adenosine
  modified_base (16)..(16): 2′-O-methyl-uridine
  modified_base (18)..(18): 2′-O-methyl-guanosine
  modified_base (20)..(20): 2′-O-methyl-guanosine
  modified_base (22)..(22): 2′-O-methyl-uridine
  misc_feature (23)..(25): joined through phosphorothioate bonds
  misc_feature (23)..(23): Cholesterol conjugate with a 6 carbon linker
aacacugauuucaaauggugcuauu

SEQ ID NO: 21 - RNA, 23 nt, Artificial Sequence
  miR-29b mimic second/ sense/ passenger strand
  modified_base (1)..(1): 2′-O-methyl-adenosine
  modified_base (2)..(2): 2′-O-methyl-adenosine
  modified_base (3)..(3): 2′-O-methyl-cytidine
  modified_base (5)..(5): 2′-O-methyl-cytidine
  modified_base (6)..(6): 2′-O-methyl-uridine
  modified_base (8)..(8): 2′-O-methyl-uridine
  modified_base (9)..(9): 2′-O-methyl-uridine
  modified_base (10)..(10): 2′-O-methyl-uridine
  modified_base (12)..(12): 2′-O-methyl-cytidine
  modified_base (16)..(16): 2′-O-methyl-uridine
  modified_base (19)..(19): 2′-O-methyl-uridine
  modified_base (20)..(20): 2′-O-methyl-cytidine
  modified_base (21)..(21): 2′-O-methyl-cytidine
  modified_base (22)..(22): 2′-O-methyl-uridine
  misc_feature (23)..(23): Cholesterol conjugate with a 6 carbon linker
aacacuguuuacaaaugguccua

SEQ ID NO: 22 - RNA, 23 nt, Artificial Sequence
  miR-29b mimic second/ sense/ passenger strand
  modified_base (1)..(1): 2′-O-methyl-adenosine
  modified_base (2)..(2): 2′-O-methyl-adenosine
aacacugauuucaaauggugcua

SEQ ID NO: 23 - RNA, 25 nt, Artificial Sequence
  miR-29b mimic first/ antisense/ guide strand
  misc_feature (1)..(1): 5′-terminal monophosphate
uagcaccauuugaaaucaguguuuu

SEQ ID NO: 24 - RNA, 25 nt, Artificial Sequence
  miR-29b mimic first/ antisense/ guide strand
  misc_feature (1)..(1): 5′-terminal monophosphate
  modified_base (1)..(1): 2′-fluoro-uridine
  modified_base (4)..(4): 2′-fluoro-cytidine
  modified_base (6)..(6): 2′-fluoro-cytidine
  modified_base (7)..(7): 2′-fluoro-cytidine
  modified_base (9)..(9): 2′-fluoro-uridine
  modified_base (10)..(10): 2′-fluoro-uridine
  modified_base (11)..(11): 2′-fluoro-uridine
  modified_base (16)..(16): 2′-fluoro-uridine
  modified_base (17)..(17): 2′-fluoro-cytidine
  modified_base (20)..(20): 2′-fluoro-uridine
  modified_base (22)..(22): 2′-fluoro-uridine
  misc_feature (23)..(25): joined through phosphorothioate bonds
  modified_base (23)..(23): 2′-fluoro-uridine
uagcaccauuugaaaucaguguuuu

SEQ ID NO: 25 - RNA, 25 nt, Artificial Sequence
  miR-29b mimic first/ antisense/ guide strand
  modified_base (1)..(1): 2′-fluoro-uridine
  modified_base (4)..(4): 2′-fluoro-cytidine
  modified_base (6)..(6): 2′-fluoro-cytidine
  modified_base (7)..(7): 2′-fluoro-cytidine
  modified_base (9)..(9): 2′-fluoro-uridine
  modified_base (10)..(10): 2′-fluoro-uridine
  modified_base (11)..(11): 2′-fluoro-uridine
  modified_base (16)..(16): 2′-fluoro-uridine
  modified_base (17)..(17): 2′-fluoro-cytidine
  modified_base (20)..(20): 2′-fluoro-uridine
  modified_base (22)..(22): 2′-fluoro-uridine
  misc_feature (23)..(25): joined through phosphorothioate bonds
  modified_base (23)..(23): 2′-fluoro-uridine
uagcaccauuugaaaucaguguuuu

SEQ ID NO: 26 - RNA, 25 nt, Artificial Sequence
  miR-29b mimic first/ antisense/ guide strand
  misc_feature (1)..(1): 5′-terminal monophosphate
  modified_base (1)..(1): 2′-fluoro-uridine
  modified_base (4)..(4): 2′-fluoro-cytidine
  modified_base (6)..(6): 2′-fluoro-cytidine
  modified_base (7)..(7): 2′-fluoro-cytidine
  modified_base (9)..(9): 2′-fluoro-cytidine
  modified_base (10)..(10): 2′-fluoro-cytidine
  modified_base (11)..(11): 2′-fluoro-cytidine
  modified_base (16)..(16): 2′-fluoro-uridine
  modified_base (17)..(17): 2′-fluoro-cytidine
  modified_base (20)..(20): 2′-fluoro-uridine
  modified_base (22)..(22): 2′-fluoro-uridine
  misc_feature (23)..(25): joined through phosphorothioate bonds
  modified_base (23)..(23): 2′-fluoro-uridine
uagcaccacccgaaaucaguguuuu

SEQ ID NO: 27 - RNA, 25 nt, Artificial Sequence
  miR-29b mimic first/ antisense/ guide strand
uagcaccauuugaaaucaguguuuu

SEQ ID NO: 28 - RNA, 23 nt, Artificial Sequence
  miR-29b mimic first/ antisense/ guide strand
  modified_base (1)..(1): 2′-O-methyl-uridine
  modified_base (3)..(3): 2′-O-methyl-guanosine
  modified_base (5)..(5): 2′-O-methyl-adenosine
  modified_base (7)..(7): 2′-O-methyl-cytidine
  modified_base (9)..(9): 2′-O-methyl-uridine
  modified_base (11)..(11): 2′-O-methyl-uridine
  modified_base (13)..(13): 2′-O-methyl-adenosine
  modified_base (15)..(15): 2′-O-methyl-adenosine
  modified_base (17)..(17): 2′-O-methyl-cytidine
  modified_base (19)..(19): 2′-O-methyl-guanosine
  modified_base (21)..(21): 2′-O-methyl-guanosine
  modified_base (23)..(23): 2′-O-methyl-uridine
uagcaccauuugaaaucaguguu

SEQ ID NO: 29 - RNA, 25 nt, Artificial Sequence
  miR-29b mimic first/ antisense/ guide strand
  modified_base (1)..(1): 2′-O-methyl-uridine
  modified_base (3)..(3): 2′-O-methyl-guanosine
  modified_base (5)..(5): 2′-O-methyl-adenosine
  modified_base (7)..(7): 2′-O-methyl-cytidine
  modified_base (9)..(9): 2′-O-methyl-uridine
  modified_base (11)..(11): 2′-O-methyl-uridine
  modified_base (13)..(13): 2′-O-methyl-adenosine
  modified_base (15)..(15): 2′-O-methyl-adenosine
  modified_base (17)..(17): 2′-O-methyl-cytidine
  modified_base (19)..(19): 2′-O-methyl-guanosine
  modified_base (21)..(21): 2′-O-methyl-guanosine
  misc_feature (23)..(25): joined through phosphorothioate bonds
  modified_base (23)..(23): 2′-O-methyl-uridine
uagcaccauuugaaaucaguguuuu

SEQ ID NO: 30 - RNA, 25 nt, Artificial Sequence
  miR-29b mimic first/ antisense/ guide strand
  modified_base (1)..(1): 2′-O-methyl-uridine
  modified_base (4)..(4): 2′-O-methyl-cytidine
  modified_base (6)..(6): 2′-O-methyl-cytidine
  modified_base (7)..(7): 2′-O-methyl-cytidine
  modified_base (9)..(9): 2′-O-methyl-uridine
  modified_base (10)..(10): 2′-O-methyl-uridine
  modified_base (11)..(11): 2′-O-methyl-uridine
  modified_base (16)..(16): 2′-O-methyl-uridine
  modified_base (17)..(17): 2′-O-methyl-cytidine
  modified_base (20)..(20): 2′-O-methyl-uridine
  modified_base (22)..(22): 2′-O-methyl-uridine
  misc_feature (23)..(25): joined through phosphorothioate bonds
  modified_base (23)..(23): 2′-O-methyl-uridine
uagcaccauuugaaaucaguguuuu

SEQ ID NO: 31 - RNA, 23 nt, Artificial Sequence
  miR-29b mimic first/ antisense/ guide strand
  modified_base (1)..(1): 2′-fluoro-uridine
  modified_base (4)..(4): 2′-fluoro-cytidine
  modified_base (6)..(6): 2′-fluoro-cytidine
  modified_base (7)..(7): 2′-fluoro-cytidine
  modified_base (9)..(9): 2′-fluoro-uridine
  modified_base (10)..(10): 2′-fluoro-uridine
  modified_base (11)..(11): 2′-fluoro-uridine
  modified_base (16)..(16): 2′-fluoro-uridine
  modified_base (17)..(17): 2′-fluoro-cytidine
  modified_base (20)..(20): 2′-fluoro-uridine
  modified_base (22)..(22): 2′-fluoro-uridine
  modified_base (23)..(23): 2′-fluoro-uridine
uagcaccauuugaaaucaguguu

SEQ ID NO: 32 - RNA, 25 nt, Artificial Sequence
  miR-29b mimic first/ antisense/ guide strand
uagcaccauuugaaaucaguguuuu

SEQ ID NO: 33 - RNA, 24 nt, Artificial Sequence
  miR-29c mimic second/ sense/ passenger strand
  modified_base (1)..(1): 2′-O-methyl-uridine
  modified_base (2)..(2): 2′-O-methyl-adenosine
uaaccgauuucaaauggugcuauu

SEQ ID NO: 34 - RNA, 22 nt, Artificial Sequence
  miR-29c mimic second/ sense/ passenger strand
  modified_base (1)..(1): 2′-O-methyl-uridine
  modified_base (2)..(2): 2′-O-methyl-adenosine
  modified_base (4)..(4): 2′-O-methyl-cytidine
  modified_base (5)..(5): 2′-O-methyl-cytidine
  modified_base (7)..(7): 2′-O-methyl-uridine
  modified_base (8)..(8): 2′-O-methyl-uridine
  modified_base (9)..(9): 2′-O-methyl-uridine
  modified_base (11)..(11): 2′-O-methyl-cytidine
  modified_base (15)..(15): 2′-O-methyl-uridine
  modified_base (18)..(18): 2′-O-methyl-uridine
  modified_base (19)..(19): 2′-O-methyl-cytidine
  modified_base (20)..(20): 2′-O-methyl-cytidine
  modified_base (21)..(21): 2′-O-methyl-uridine
uaaccguuuacaaaugguccua

SEQ ID NO: 35 - RNA, 22 nt, Artificial Sequence
  miR-29c mimic second/ sense/ passenger strand
  modified_base (1)..(1): 2′-O-methyl-uridine
  modified_base (2)..(2): 2′-O-methyl-adenosine
  modified_base (4)..(4): 2′-O-methyl-cytidine
  modified_base (5)..(5): 2′-O-methyl-cytidine
  modified_base (7)..(7): 2′-O-methyl-uridine
  modified_base (8)..(8): 2′-O-methyl-uridine
  modified_base (9)..(9): 2′-O-methyl-uridine
  modified_base (11)..(11): 2′-O-methyl-cytidine
  modified_base (15)..(15): 2′-O-methyl-uridine
  modified_base (18)..(18): 2′-O-methyl-uridine
  modified_base (19)..(19): 2′-O-methyl-cytidine
  modified_base (20)..(20): 2′-O-methyl-cytidine
  modified_base (21)..(21): 2′-O-methyl-uridine
  misc_feature (22)..(22): Cholesterol conjugate with a 6 carbon linker
uaaccguuuacaaaugguccua

SEQ ID NO: 36 - RNA, 24 nt, Artificial Sequence
  miR-29c mimic second/ sense/ passenger strand
  modified_base (1)..(1): 2′-O-methyl-uridine
  modified_base (2)..(2): 2′-O-methyl-adenosine
  misc_feature (22)..(24): joined through phosphorothioate bonds
  misc_feature (24)..(24): Cholesterol conjugate with a 6 carbon linker via phosphorothioate bond
uaaccgauuucaaauggugcuauu

SEQ ID NO: 37 - RNA, 22 nt, Artificial Sequence
  miR-29c mimic second/ sense/ passenger strand
  modified_base (1)..(1): 2′-O-methyl-uridine
  modified_base (2)..(2): 2′-O-methyl-adenosine
uaaccgauuucaaauggugcua

SEQ ID NO: 38 - RNA, 24 nt, Artificial Sequence
  miR-29c mimic first/ antisense/ guide strand
  misc_feature (1)..(1): 5′-terminal monophosphate
uagcaccauuugaaaucgguuauu

SEQ ID NO: 39 - RNA, 24 nt, Artificial Sequence
  miR-29c mimic first/ antisense/ guide strand
  misc_feature (1)..(1): 5′-terminal monophosphate
  modified_base (1)..(1): 2′-fluoro-uridine
  modified_base (4)..(4): 2′-fluoro-cytidine
  modified_base (6)..(6): 2′-fluoro-cytidine
  modified_base (7)..(7): 2′-fluoro-cytidine
  modified_base (9)..(9): 2′-fluoro-uridine
  modified_base (10)..(10): 2′-fluoro-uridine
  modified_base (11)..(11): 2′-fluoro-uridine
  modified_base (16)..(16): 2′-fluoro-uridine
  modified_base (17)..(17): 2′-fluoro-cytidine
  modified_base (20)..(20): 2′-fluoro-uridine
  modified_base (21)..(21): 2′-fluoro-uridine
  misc_feature (22)..(24): joined through phosphorothioate bonds
uagcaccauuugaaaucgguuauu

SEQ ID NO: 40 - RNA, 24 nt, Artificial Sequence
  miR-29c mimic first/ antisense/ guide strand
  modified_base (1)..(1): 2′-fluoro-uridine
  modified_base (4)..(4): 2′-fluoro-cytidine
  modified_base (6)..(6): 2′-fluoro-cytidine
  modified_base (7)..(7): 2′-fluoro-cytidine
  modified_base (9)..(9): 2′-fluoro-uridine
  modified_base (10)..(10): 2′-fluoro-uridine
  modified_base (11)..(11): 2′-fluoro-uridine
  modified_base (16)..(16): 2′-fluoro-uridine
  modified_base (17)..(17): 2′-fluoro-cytidine
  modified_base (20)..(20): 2′-fluoro-uridine
  modified_base (21)..(21): 2′-fluoro-uridine
  misc_feature (22)..(24): joined through phosphorothioate bonds
uagcaccauuugaaaucgguuauu

SEQ ID NO: 41 - RNA, 24 nt, Artificial Sequence
  miR-29c mimic first/ antisense/ guide strand
uagcaccauuugaaaucgguuauu

SEQ ID NO: 42 - RNA, 23 nt, Artificial Sequence
  miR-29 mimic sense/ passenger/ second strand
  misc_feature (1)..(3): joined through phosphorothioate bonds
  modified_base (1)..(1): 2′-fluoro-adenosine
  modified_base (2)..(2): 2′-O-methyl-adenosine
  modified_base (3)..(3): 2′-fluoro-cytidine
  modified_base (4)..(4): 2′-O-methyl-adenosine
  modified_base (5)..(5): 2′-fluoro-cytidine
  modified_base (6)..(6): 2′-O-methyl-uridine
  modified_base (7)..(7): 2′-fluoro-guanosine
  modified_base (8)..(8): 2′-O-methyl-uridine
  modified_base (9)..(9): 2′-fluoro-uridine
  modified_base (10)..(10): 2′-fluoro-uridine
  modified_base (11)..(11): 2′-fluoro-adenosine
  modified_base (12)..(12): 2′-O-methyl-cytidine
  modified_base (13)..(13): 2′-fluoro-adenosine
  modified_base (14)..(14): 2′-O-methyl-adenosine
  modified_base (15)..(15): 2′-fluoro-adenosine
  modified_base (16)..(16): 2′-O-methyl-uridine
  modified_base (17)..(17): 2′-fluoro-guanosine
  modified_base (18)..(18): 2′-O-methyl-guanosine
  modified_base (19)..(19): 2′-fluoro-uridine
  modified_base (20)..(20): 2′-O-methyl-cytidine
  modified_base (21)..(21): 2′-fluoro-cytidine
  modified_base (22)..(22): 2′-O-methyl-uridine
  modified_base (23)..(23): 2′-fluoro-adenosine
  misc_feature (23)..(23): Three GalNac conjugates linked via phosphorothioate bonds
aacacuguuuacaaaugguccua

SEQ ID NO: 43 - RNA, 23 nt, Artificial Sequence
  miR-29 mimic sense/ passenger/ second strand
  misc_feature (1)..(3): joined through phosphorothioate bonds
  modified_base (1)..(1): 2′-fluoro-adenosine
  modified_base (2)..(2): 2′-O-methyl-adenosine
  modified_base (3)..(3): 2′-fluoro-cytidine
  modified_base (4)..(4): 2′-O-methyl-adenosine
  modified_base (5)..(5): 2′-fluoro-cytidine
  modified_base (6)..(6): 2′-O-methyl-uridine
  modified_base (7)..(7): 2′-fluoro-guanosine
  modified_base (8)..(8): 2′-O-methyl-uridine
  modified_base (9)..(9): 2′-fluoro-uridine
  modified_base (10)..(10): 2′-O-methyl-uridine
  modified_base (11)..(11): 2′-fluoro-adenosine
  modified_base (12)..(12): 2′-fluoro-cytidine
  modified_base (13)..(13): 2′-fluoro-adenosine
  modified_base (14)..(14): 2′-O-methyl-adenosine
  modified_base (15)..(15): 2′-fluoro-adenosine
  modified_base (16)..(16): 2′-O-methyl-uridine
  modified_base (17)..(17): 2′-fluoro-guanosine
  modified_base (18)..(18): 2′-O-methyl-guanosine
  modified_base (19)..(19): 2′-fluoro-uridine
  modified_base (20)..(20): 2′-O-methyl-cytidine
  modified_base (21)..(21): 2′-fluoro-cytidine
  modified_base (22)..(22): 2′-O-methyl-uridine
  modified_base (23)..(23): 2′-fluoro-adenosine
  misc_feature (23)..(23): Three GalNac conjugates linked via phosphorothioate bonds
aacacuguuuacaaaugguccua

SEQ ID NO: 44 - RNA, 23 nt, Artificial Sequence
  miR-29 mimic sense/ passenger/ second strand
  misc_feature (1)..(3): joined through phosphorothioate bonds
  modified_base (1)..(1): 2′-fluoro-adenosine
  modified_base (2)..(2): 2′-O-methyl-adenosine
  modified_base (3)..(3): 2′-fluoro-cytidine
  modified_base (4)..(4): 2′-O-methyl-adenosine
  modified_base (5)..(5): 2′-fluoro-cytidine
  modified_base (6)..(6): 2′-O-methyl-uridine
  modified_base (7)..(7): 2′-fluoro-guanosine
  modified_base (8)..(8): 2′-O-methyl-uridine
  modified_base (9)..(9): 2′-fluoro-uridine
  modified_base (10)..(10): 2′-fluoro-uridine
  modified_base (11)..(11): 2′-fluoro-adenosine
  modified_base (12)..(12): 2′-O-methyl-cytidine
  modified_base (13)..(13): 2′-fluoro-adenosine
  modified_base (14)..(14): 2′-O-methyl-adenosine
  modified_base (15)..(15): 2′-fluoro-adenosine
  modified_base (16)..(16): 2′-O-methyl-uridine
  modified_base (17)..(17): 2′-fluoro-guanosine
  modified_base (18)..(18): 2′-O-methyl-guanosine
  modified_base (19)..(19): 2′-fluoro-uridine
  modified_base (20)..(20): 2′-O-methyl-cytidine
  modified_base (21)..(21): 2′-fluoro-cytidine
  modified_base (22)..(22): 2′-O-methyl-uridine
  modified_base (23)..(23): 2′-fluoro-adenosine
aacacuguuuacaaaugguccua

SEQ ID NO: 45 - RNA, 23 nt, Artificial Sequence
  miR-29 mimic sense/ passenger/ second strand
  misc_feature (1)..(3): joined through phosphorothioate bonds
  modified_base (1)..(1): 2′-fluoro-adenosine
  modified_base (2)..(2): 2′-O-methyl-adenosine
  modified_base (3)..(3): 2′-fluoro-cytidine
  modified_base (4)..(4): 2′-O-methyl-adenosine
  modified_base (5)..(5): 2′-fluoro-cytidine
  modified_base (6)..(6): 2′-O-methyl-uridine
  modified_base (7)..(7): 2′-fluoro-guanosine
  modified_base (8)..(8): 2′-O-methyl-uridine
  modified_base (9)..(9): 2′-fluoro-uridine
  modified_base (10)..(10): 2′-O-methyl-uridine
  modified_base (11)..(11): 2′-fluoro-adenosine
  modified_base (12)..(12): 2′-fluoro-cytidine
  modified_base (13)..(13): 2′-fluoro-adenosine
  modified_base (14)..(14): 2′-O-methyl-adenosine
  modified_base (15)..(15): 2′-fluoro-adenosine
  modified_base (16)..(16): 2′-O-methyl-uridine
  modified_base (17)..(17): 2′-fluoro-guanosine
  modified_base (18)..(18): 2′-O-methyl-guanosine
  modified_base (19)..(19): 2′-fluoro-uridine
  modified_base (20)..(20): 2′-O-methyl-cytidine
  modified_base (21)..(21): 2′-fluoro-cytidine
  modified_base (22)..(22): 2′-O-methyl-uridine
  modified_base (23)..(23): 2′-fluoro-adenosine
aacacuguuuacaaaugguccua

SEQ ID NO: 46 - RNA, 23 nt, Artificial Sequence
  miR-29 mimic sense/ passenger/ second strand
  misc_feature (1)..(3): joined through phosphorothioate bonds
  modified_base (1)..(1): 2′-fluoro-adenosine
  modified_base (2)..(2): 2′-O-methyl-adenosine
  modified_base (3)..(3): 2′-fluoro-cytidine
  modified_base (4)..(4): 2′-O-methyl-adenosine
  modified_base (5)..(5): 2′-fluoro-cytidine
  modified_base (6)..(6): 2′-O-methyl-uridine
  modified_base (7)..(7): 2′-fluoro-guanosine
  modified_base (8)..(8): 2′-O-methyl-uridine
  modified_base (9)..(9): 2′-fluoro-uridine
  modified_base (10)..(10): 2′-O-methyl-uridine
  modified_base (11)..(11): 2′-fluoro-adenosine
  modified_base (12)..(12): 2′-fluoro-cytidine
  modified_base (13)..(13): 2′-fluoro-adenosine
  modified_base (14)..(14): 2′-O-methyl-adenosine
  modified_base (15)..(15): 2′-fluoro-adenosine
  modified_base (16)..(16): 2′-O-methyl-uridine
  modified_base (17)..(17): 2′-fluoro-guanosine
  modified_base (18)..(18): 2′-O-methyl-guanosine
  modified_base (19)..(19): 2′-fluoro-uridine
  modified_base (20)..(20): 2′-O-methyl-cytidine
  modified_base (21)..(21): 2′-fluoro-cytidine
  modified_base (22)..(22): 2′-O-methyl-uridine
  modified_base (23)..(23): 2′-fluoro-adenosine
  misc_feature (23)..(23): Three GalNac conjugates linked via phosphorothioate bonds
aacacuguuuacaaaugguccua

SEQ ID NO: 47 - RNA, 23 nt, Artificial Sequence
  miR-29 mimic sense/ passenger/ second strand
  misc_feature (1)..(3): joined through phosphorothioate bonds
  modified_base (1)..(1): 2′-fluoro-adenosine
  modified_base (2)..(2): 2′-O-methyl-adenosine
  modified_base (3)..(3): 2′-fluoro-cytidine
  modified_base (4)..(4): 2′-O-methyl-adenosine
  modified_base (5)..(5): 2′-fluoro-cytidine
  modified_base (6)..(6): 2′-O-methyl-uridine
  modified_base (7)..(7): 2′-fluoro-guanosine
  modified_base (8)..(8): 2′-O-methyl-uridine
  modified_base (9)..(9): 2′-fluoro-uridine
  modified_base (10)..(10): 2′-O-methyl-uridine
  modified_base (11)..(11): 2′-fluoro-adenosine
  modified_base (12)..(12): 2′-O-methyl-cytidine
  modified_base (13)..(13): 2′-fluoro-adenosine
  modified_base (14)..(14): 2′-fluoro-adenosine
  modified_base (15)..(15): 2′-fluoro-adenosine
  modified_base (16)..(16): 2′-O-methyl-uridine
  modified_base (17)..(17): 2′-fluoro-guanosine
  modified_base (18)..(18): 2′-O-methyl-guanosine
  modified_base (19)..(19): 2′-fluoro-uridine
  modified_base (20)..(20): 2′-O-methyl-cytidine
  modified_base (21)..(21): 2′-fluoro-cytidine
  modified_base (22)..(22): 2′-O-methyl-uridine
  modified_base (23)..(23): 2′-fluoro-adenosine
aacacuguuuacaaaugguccua

SEQ ID NO: 48 - RNA, 23 nt, Artificial Sequence
  miR-29 mimic sense/ passenger/ second strand
  misc_feature (1)..(3): joined through phosphorothioate bonds
  modified_base (1)..(1): 2′-fluoro-adenosine
  modified_base (2)..(2): 2′-O-methyl-adenosine
  modified_base (3)..(3): 2′-fluoro-cytidine
  modified_base (4)..(4): 2′-O-methyl-adenosine
  modified_base (5)..(5): 2′-fluoro-cytidine
  modified_base (6)..(6): 2′-O-methyl-uridine
  modified_base (7)..(7): 2′-fluoro-guanosine
  modified_base (8)..(8): 2′-fluoro-uridine
  modified_base (9)..(9): 2′-fluoro-uridine
  modified_base (10)..(10): 2′-O-methyl-uridine
  modified_base (11)..(11): 2′-fluoro-adenosine
  modified_base (12)..(12): 2′-O-methyl-cytidine
  modified_base (13)..(13): 2′-fluoro-adenosine
  modified_base (14)..(14): 2′-fluoro-adenosine
  modified_base (15)..(15): 2′-fluoro-adenosine
  modified_base (16)..(16): 2′-O-methyl-uridine
  modified_base (17)..(17): 2′-fluoro-guanosine
  modified_base (18)..(18): 2′-O-methyl-guanosine
  modified_base (19)..(19): 2′-fluoro-uridine
  modified_base (20)..(20): 2′-O-methyl-cytidine
  modified_base (21)..(21): 2′-fluoro-cytidine
  modified_base (22)..(22): 2′-O-methyl-uridine
  modified_base (23)..(23): 2′-fluoro-adenosine
aacacuguuuacaaaugguccua

SEQ ID NO: 49 - RNA, 23 nt, Artificial Sequence
  miR-29 mimic sense/ passenger/ second strand
  misc_feature (1)..(3): joined through phosphorothioate bonds
  modified_base (1)..(1): deoxyadenosine
  modified_base (2)..(2): 2′-O-methyl-adenosine
  modified_base (3)..(3): 2′-fluoro-cytidine
  modified_base (4)..(4): 2′-O-methyl-adenosine
  modified_base (5)..(5): 2′-fluoro-cytidine
  modified_base (6)..(6): 2′-O-methyl-uridine
  modified_base (7)..(7): deoxyguanosine
  modified_base (8)..(8): 2′-O-methyl-uridine
  modified_base (9)..(9): 2′-fluoro-uridine
  modified_base (10)..(10): 2′-O-methyl-uridine
  modified_base (11)..(11): deoxyadenosine
  modified_base (12)..(12): 2′-fluoro-cytidine
  modified_base (13)..(13): deoxyadenosine
  modified_base (14)..(14): 2′-O-methyl-adenosine
  modified_base (15)..(15): deoxyadenosine
  modified_base (16)..(16): 2′-O-methyl-uridine
  modified_base (17)..(17): deoxyguanosine
  modified_base (18)..(18): 2′-O-methyl-guanosine
  modified_base (19)..(19): 2′-fluoro-uridine
  modified_base (20)..(20): 2′-O-methyl-cytidine
  modified_base (21)..(21): 2′-fluoro-cytidine
  misc_feature (22)..(23): joined through phosphorothioate bonds
  modified_base (22)..(22): 2′-O-methyl-uridine
  modified_base (23)..(23): deoxyadenosine
aacacuguuuacaaaugguccua

SEQ ID NO: 50 - RNA, 23 nt, Artificial Sequence
  miR-29 mimic sense/ passenger/ second strand
  misc_feature (1)..(3): joined through phosphorothioate bonds
  modified_base (1)..(1): 2′-O-methyl-adenosine
  modified_base (2)..(2): deoxyadenosine
  modified_base (3)..(3): 2′-O-methyl-cytidine
  modified_base (4)..(4): deoxyadenosine
  modified_base (5)..(5): 2′-O-methyl-cytidine
  modified_base (6)..(6): 2′-fluoro-uridine
  modified_base (7)..(7): 2′-O-methyl-guanosine
  modified_base (8)..(8): 2′-fluoro-uridine
  modified_base (9)..(9): 2′-O-methyl-uridine
  modified_base (10)..(10): 2′-fluoro-uridine
  modified_base (11)..(11): 2′-O-methyl-adenosine
  modified_base (12)..(12): 2′-fluoro-cytidine
  modified_base (13)..(13): deoxyadenosine
  modified_base (14)..(14): deoxyadenosine
  modified_base (15)..(15): 2′-O-methyl-adenosine
  modified_base (16)..(16): 2′-fluoro-uridine
  modified_base (17)..(17): 2′-O-methyl-guanosine
  modified_base (18)..(18): deoxyguanosine
  modified_base (19)..(19): 2′-O-methyl-uridine
  modified_base (20)..(20): 2′-fluoro-cytidine
  modified_base (21)..(21): 2′-O-methyl-cytidine
  misc_feature (22)..(23): joined through phosphorothioate bonds
  modified_base (22)..(22): 2′-fluoro-uridine
  modified_base (23)..(23): deoxyadenosine
aacacuguuuacaaaugguccua

SEQ ID NO: 51 - RNA, 23 nt, Artificial Sequence
  miR-29 mimic sense/ passenger/ second strand
  misc_feature (1)..(3): joined through phosphorothioate bonds
  modified_base (1)..(1): 2′-O-methyl-adenosine
  modified_base (2)..(2): deoxyadenosine
  modified_base (3)..(3): 2′-O-methyl-cytidine
  modified_base (4)..(4): deoxyadenosine
  modified_base (5)..(5): 2′-O-methyl-cytidine
  modified_base (6)..(6): 2′-fluoro-uridine
  modified_base (7)..(7): 2′-O-methyl-guanosine
  modified_base (8)..(8): 2′-fluoro-uridine
  modified_base (9)..(9): 2′-O-methyl-uridine
  modified_base (10)..(10): 2′-fluoro-uridine
  misc_feature (11)..(14): joined through phosphorothioate bonds
  modified_base (11)..(11): 2′-O-methyl-adenosine
  modified_base (12)..(12): 2′-fluoro-cytidines
  modified_base (13)..(13): deoxyadenosine
  modified_base (14)..(14): deoxyadenosine
  modified_base (15)..(15): 2′-O-methyl-adenosine
  modified_base (16)..(16): 2′-fluoro-uridine
  modified_base (17)..(17): 2′-O-methyl-guanosine
  modified_base (18)..(18): deoxyguanosine
  modified_base (19)..(19): 2′-O-methyl-uridine
  modified_base (20)..(20): 2′-fluoro-cytidine
  modified_base (21)..(21): 2′-O-methyl-cytidine
  misc_feature (22)..(23): joined through phosphorothioate bonds
  modified_base (22)..(22): 2′-fluoro-uridine
  modified_base (23)..(23): deoxyadenosine
aacacuguuuacaaaugguccua

SEQ ID NO: 52 - RNA, 23 nt, Artificial Sequence
  miR-29 mimic sense/ passenger/ second strand
  misc_feature (1)..(3): joined through phosphorothioate bonds
  modified_base (1)..(1): 2′-fluoro-adenosine
  modified_base (2)..(2): 2′-O-methyl-adenosine
  modified_base (3)..(3): 2′-fluoro-cytidine
  modified_base (4)..(4): 2′-O-methyl-adenosine
  modified_base (5)..(5): 2′-fluoro-cytidine
  modified_base (6)..(6): 2′-O-methyl-uridine
  modified_base (7)..(7): 2′-fluoro-guanosine
  modified_base (8)..(8): 2′-O-methyl-uridine
  modified_base (9)..(9): 2′-fluoro-uridine
  modified_base (10)..(10): 2′-O-methyl-uridine
  modified_base (11)..(11): 2′-fluoro-adenosine
  modified_base (12)..(12): 2′-fluoro-cytidine
  modified_base (13)..(13): 2′-fluoro-adenosine
  modified_base (14)..(14): 2′-O-methyl-adenosine
  modified_base (15)..(15): 2′-fluoro-adenosine
  modified_base (16)..(16): 2′-O-methyl-uridine
  modified_base (17)..(17): 2′-fluoro-guanosine
  modified_base (18)..(18): 2′-O-methyl-guanosine
  modified_base (19)..(19): 2′-fluoro-uridine
  modified_base (20)..(20): 2′-O-methyl-cytidine
  modified_base (21)..(21): 2′-fluoro-cytidine
  modified_base (22)..(22): 2′-O-methyl-uridine
  modified_base (23)..(23): 2′-fluoro-adenosine
  misc_feature (23)..(23): Cholesterol conjugate with a 6 carbon linker
aacacuguuuacaaaugguccua

SEQ ID NO: 53 - RNA, 23 nt, Artificial Sequence
  miR-29 mimic sense/ passenger/ second strand
  misc_feature (1)..(3): joined through phosphorothioate bonds
  modified_base (1)..(1): 2′-fluoro-adenosine
  modified_base (2)..(2): 2′-O-methyl-adenosine
  modified_base (3)..(3): 2′-fluoro-cytidine
  modified_base (4)..(4): 2′-O-methyl-adenosine
  modified_base (5)..(5): 2′-fluoro-cytidine
  modified_base (6)..(6): 2′-O-methyl-uridine
  modified_base (7)..(7): 2′-fluoro-guanosine
  modified_base (8)..(8): 2′-O-methyl-uridine
  modified_base (9)..(9): 2′-fluoro-uridine
  modified_base (10)..(10): 2′-O-methyl-uridine
  modified_base (11)..(11): 2′-fluoro-adenosine
  modified_base (12)..(12): 2′-fluoro-cytidine
  modified_base (13)..(13): 2′-fluoro-adenosine
  modified_base (14)..(14): 2′-O-methyl-adenosine
  modified_base (15)..(15): 2′-fluoro-adenosine
  modified_base (16)..(16): 2′-O-methyl-uridine
  modified_base (17)..(17): 2′-fluoro-guanosine
  modified_base (18)..(18): 2′-O-methyl-guanosine
  modified_base (19)..(19): 2′-fluoro-uridine
  modified_base (20)..(20): 2′-O-methyl-cytidine
  modified_base (21)..(21): 2′-fluoro-cytidine
  modified_base (22)..(22): 2′-O-methyl-uridine
  modified_base (23)..(23): 2′-fluoro-adenosine
  misc_feature (23)..(23): Cholesterol conjugate with a 6 carbon linker via phosphorothioate bond
aacacuguuuacaaaugguccua

SEQ ID NO: 54 - RNA, 23 nt, Artificial Sequence
  miR-29 mimic sense/ passenger/ second strand
  modified_base (1)..(1): 2′-O-methyl-adenosine
  modified_base (2)..(2): 2′-O-methyl-adenosine
  modified_base (3)..(3): 2′-O-methyl-cytidine
  modified_base (5)..(5): 2′-O-methyl-cytidine
  modified_base (6)..(6): 2′-O-methyl-uridine
  modified_base (8)..(8): 2′-O-methyl-uridine
  modified_base (9)..(9): 2′-O-methyl-uridine
  modified_base (10)..(10): 2′-O-methyl-uridine
  modified_base (12)..(12): 2′-O-methyl-cytidine
  modified_base (16)..(16): 2′-O-methyl-uridine
  modified_base (19)..(19): 2′-O-methyl-uridine
  modified_base (20)..(20): 2′-O-methyl-cytidine
  modified_base (21)..(21): 2′-O-methyl-cytidine
  modified_base (22)..(22): 2′-O-methyl-uridine
  misc_feature (23)..(23): Cholesterol conjugate with a 6 carbon linker
aacacuguuuacaaaugguccua

SEQ ID NO: 55 - RNA, 25 nt, Artificial Sequence
  miR-29 mimic antisense/ guide/ first strand
  misc_feature (1)..(3): joined through phosphorothioate bonds
  modified_base (1)..(1): 2′-O-methyl-uridine
  modified_base (2)..(2): 2′-fluoro-adenosine
  modified_base (3)..(3): 2′-O-methyl-guanosine
  modified_base (4)..(4): 2′-fluoro-cytidine
  modified_base (5)..(5): 2′-O-methyl-adenosine
  modified_base (6)..(6): 2′-fluoro-cytidine
  modified_base (7)..(7): 2′-O-methyl-cytidine
  modified_base (8)..(8): 2′-fluoro-adenosine
  modified_base (9)..(9): 2′-O-methyl-uridine
  modified_base (10)..(10): 2′-fluoro-uridine
  modified_base (11)..(11): 2′-O-methyl-uridine
  modified_base (12)..(12): 2′-O-methyl-guanosine
  modified_base (13)..(13): 2′-O-methyl-adenosine
  modified_base (14)..(14): 2′-fluoro-adenosine
  modified_base (15)..(15): 2′-O-methyl-adenosine
  modified_base (16)..(16): 2′-fluoro-uridine
  modified_base (17)..(17): 2′-O-methyl-cytidine
  modified_base (18)..(18): 2′-fluoro-adenosine
  modified_base (19)..(19): 2′-O-methyl-guanosine
  modified_base (20)..(20): 2′-fluoro-uridine
  modified_base (21)..(21): 2′-O-methyl-guanosine
  modified_base (22)..(22): 2′-fluoro-uridine
  misc_feature (23)..(25): joined through phosphorothioate bonds
  modified_base (23)..(23): 2′-O-methyl-uridine
  modified_base (24)..(24): 2′-O-methyl-uridine
  modified_base (25)..(25): 2′-O-methyl-uridine
uagcaccauuugaaaucaguguuuu

SEQ ID NO: 56 - RNA, 25 nt, Artificial Sequence
  miR-29 mimic antisense/ guide/ first strand
  misc_feature (1)..(3): joined through phosphorothioate bonds
  modified_base (1)..(1): 2′-O-methyl-uridine
  modified_base (2)..(2): 2′-fluoro-adenosine
  modified_base (3)..(3): 2′-O-methyl-guanosine
  modified_base (4)..(4): locked nucleic acid cytidine
  modified_base (5)..(5): 2′-O-methyl-adenosine
  modified_base (6)..(6): 2′-fluoro-cytidine
  modified_base (7)..(7): 2′-O-methyl-cytidine
  modified_base (8)..(8): 2′-fluoro-adenosine
  modified_base (9)..(9): 2′-O-methyl-uridine
  modified_base (10)..(10): 2′-fluoro-uridine
  modified_base (11)..(11): 2′-O-methyl-uridine
  modified_base (12)..(12): 2′-O-methyl-guanosine
  modified_base (13)..(13): 2′-O-methyl-adenosine
  modified_base (14)..(14): 2′-fluoro-adenosine
  modified_base (15)..(15): 2′-O-methyl-adenosine
  modified_base (16)..(16): 2′-fluoro-uridine
  modified_base (17)..(17): 2′-O-methyl-cytidine
  modified_base (18)..(18): 2′-fluoro-adenosine
  modified_base (19)..(19): 2′-O-methyl-guanosine
  modified_base (20)..(20): 2′-fluoro-uridine
  modified_base (21)..(21): 2′-O-methyl-guanosine
  modified_base (22)..(22): 2′-fluoro-uridine
  misc_feature (23)..(25): joined through phosphorothioate bonds
  modified_base (23)..(23): 2′-O-methyl-uridine
  modified_base (24)..(24): 2′-O-methyl-uridine
  modified_base (25)..(25): 2′-O-methyl-uridine
uagcaccauuugaaaucaguguuuu

SEQ ID NO: 57 - RNA, 25 nt, Artificial Sequence
  miR-29 mimic antisense/ guide/ first strand
  misc_feature (1)..(3): joined through phosphorothioate bonds
  modified_base (1)..(1): 2′-O-methyl-uridine
  modified_base (2)..(2): 2′-fluoro-adenosine
  modified_base (3)..(3): 2′-O-methyl-guanosine
  modified_base (4)..(4): locked nucleic acid cytidine
  modified_base (5)..(5): 2′-O-methyl-adenosine
  modified_base (6)..(6): 2′-fluoro-cytidine
  modified_base (7)..(7): 2′-O-methyl-cytidine
  modified_base (8)..(8): 2′-fluoro-adenosine
  modified_base (9)..(9): 2′-O-methyl-uridine
  modified_base (10)..(10): 2′-fluoro-uridine
  modified_base (11)..(11): 2′-O-methyl-uridine
  modified_base (12)..(12): 2′-O-methyl-guanosine
  modified_base (13)..(13): 2′-O-methyl-adenosine
  modified_base (14)..(14): 2′-fluoro-adenosine
  modified_base (15)..(15): 2′-O-methyl-adenosine
  modified_base (16)..(16): locked nucleic acid thymidine
  modified_base (17)..(17): 2′-O-methyl-cytidine
  modified_base (18)..(18): 2′-fluoro-adenosine
  modified_base (19)..(19): 2′-O-methyl-guanosine
  modified_base (20)..(20): 2′-fluoro-uridine
  modified_base (21)..(21): 2′-O-methyl-guanosine
  modified_base (22)..(22): 2′-fluoro-uridine
  misc_feature (23)..(25): joined through phosphorothioate bonds
  modified_base (23)..(23): 2′-O-methyl-uridine
  modified_base (24)..(24): 2′-O-methyl-uridine
  modified_base (25)..(25): 2′-O-methyl-uridine
uagcaccauuugaaancaguguuuu

SEQ ID NO: 58 - RNA, 25 nt, Artificial Sequence
  miR-29 mimic antisense/ guide/ first strand
  misc_feature (1)..(1): 5′-terminal monophosphate
  modified_base (1)..(1): 2′-fluoro-uridine
  modified_base (4)..(4): 2′-fluoro-cytidine
  modified_base (6)..(6): 2′-fluoro-cytidine
  modified_base (7)..(7): 2′-fluoro-cytidine
  modified_base (9)..(9): 2′-fluoro-uridine
  modified_base (10)..(10): 2′-fluoro-uridine
  modified_base (11)..(11): 2′-fluoro-uridine
  modified_base (16)..(16): 2′-fluoro-uridine
  modified_base (17)..(17): 2′-fluoro-cytidine
  modified_base (20)..(20): 2′-fluoro-uridine
  modified_base (22)..(22): 2′-fluoro-uridine
  misc_feature (23)..(25): joined through phosphorothioate bonds
  modified_base (23)..(23): 2′-fluoro-uridine
  misc_feature (25)..(25): 3′ Fluorescein linked through phosphorothioate bond
uagcaccauuugaaaucaguguuuu

SEQ ID NO: 59 - RNA, 25 nt, Artificial Sequence
  miR-29 mimic antisense/ guide/ first strand
  misc_feature (1)..(3): joined through phosphorothioate bonds
  modified_base (1)..(1): 2′-O-methyl-uridine
  modified_base (2)..(2): 2′-fluoro-adenosine
  modified_base (3)..(3): 2′-O-methyl-guanosine
  modified_base (4)..(4): 2′-fluoro-cytidine
  modified_base (5)..(5): 2′-O-methyl-adenosine
  modified_base (6)..(6): 2′-fluoro-cytidine
  modified_base (7)..(7): 2′-O-methyl-cytidine
  modified_base (8)..(8): 2′-fluoro-adenosine
  modified_base (9)..(9): 2′-O-methyl-uridine
  modified_base (10)..(10): 2′-fluoro-uridine
  modified_base (11)..(11): 2′-O-methyl-uridine
  modified_base (12)..(12): 2′-O-methyl-guanosine
  modified_base (13)..(13): 2′-O-methyl-adenosine
  modified_base (14)..(14): 2′-fluoro-adenosine
  modified_base (15)..(15): 2′-O-methyl-adenosine
  modified_base (16)..(16): 2′-fluoro-uridine
  modified_base (17)..(17): 2′-O-methyl-cytidine
  modified_base (18)..(18): 2′-fluoro-adenosine
  modified_base (19)..(19): 2′-O-methyl-guanosine
  modified_base (20)..(20): 2′-fluoro-uridine
  modified_base (21)..(21): 2′-O-methyl-guanosine
  modified_base (22)..(22): 2′-fluoro-uridine
  misc_feature (23)..(25): joined through phosphorothioate bonds
  modified_base (23)..(23): 2′-O-methyl-uridine
  modified_base (24)..(24): 2′-O-methyl-uridine
  modified_base (25)..(25): 2′-O-methyl-uridine
  misc_feature (25)..(25): 3′ Fluorescein linked through phosphorothioate bond
uagcaccauuugaaaucaguguuuu

SEQ ID NO: 60 - RNA, 25 nt, Artificial Sequence
  miR-29 mimic antisense/ guide/ first strand
  misc_feature (1)..(3): joined through phosphorothioate bonds
  modified_base (1)..(1): 2′-O-methyl-uridine
  modified_base (2)..(2): 2′-fluoro-adenosine
  modified_base (3)..(3): 2′-O-methyl-guanosine
  modified_base (4)..(4): 2′-fluoro-cytidine
  modified_base (5)..(5): 2′-O-methyl-adenosine
  modified_base (6)..(6): 2′-fluoro-cytidine
  modified_base (7)..(7): 2′-O-methyl-cytidine
  modified_base (8)..(8): 2′-fluoro-adenosine
  modified_base (9)..(9): 2′-O-methyl-uridine
  modified_base (10)..(10): 2′-fluoro-uridine
  modified_base (11)..(11): 2′-O-methyl-uridine
  modified_base (12)..(12): 2′-fluoro-guanosine
  modified_base (13)..(13): 2′-O-methyl-adenosine
  modified_base (14)..(14): 2′-O-methyl-adenosine
  modified_base (15)..(15): 2′-O-methyl-adenosine
  modified_base (16)..(16): 2′-fluoro-uridine
  modified_base (17)..(17): 2′-O-methyl-cytidine
  modified_base (18)..(18): 2′-fluoro-adenosine
  modified_base (19)..(19): 2′-O-methyl-guanosine
  modified_base (20)..(20): 2′-fluoro-uridine
  modified_base (21)..(21): 2′-O-methyl-guanosine
  modified_base (22)..(22): 2′-fluoro-uridine
  misc_feature (23)..(25): joined through phosphorothioate bonds
  modified_base (23)..(23): 2′-O-methyl-uridine
  modified_base (24)..(24): 2′-O-methyl-uridine
  modified_base (25)..(25): 2′-O-methyl-uridine
uagcaccauuugaaaucaguguuuu

SEQ ID NO: 61 - RNA, 25 nt, Artificial Sequence
  miR-29 mimic antisense/ guide/ first strand
  misc_feature (1)..(3): joined through phosphorothioate bonds
  modified_base (1)..(1): 2′-O-methyl-uridine
  modified_base (2)..(2): 2′-fluoro-adenosine
  modified_base (3)..(3): 2′-O-methyl-guanosine
  modified_base (4)..(4): 2′-fluoro-cytidine
  modified_base (5)..(5): 2′-O-methyl-adenosine
  modified_base (6)..(6): 2′-fluoro-cytidine
  modified_base (7)..(7): 2′-O-methyl-cytidine
  modified_base (8)..(8): 2′-fluoro-adenosine
  modified_base (9)..(9): 2′-O-methyl-uridine
  modified_base (10)..(10): 2′-O-methyl-uridine
  modified_base (11)..(11): 2′-O-methyl-uridine
  modified_base (12)..(12): 2′-fluoro-guanosine
  modified_base (13)..(13): 2′-O-methyl-adenosine
  modified_base (14)..(14): 2′-fluoro-adenosine
  modified_base (15)..(15): 2′-O-methyl-adenosine
  modified_base (16)..(16): 2′-fluoro-uridine
  modified_base (17)..(17): 2′-O-methyl-cytidine
  modified_base (18)..(18): 2′-fluoro-adenosine
  modified_base (19)..(19): 2′-O-methyl-guanosine
  modified_base (20)..(20): 2′-fluoro-uridine
  modified_base (21)..(21): 2′-O-methyl-guanosine
  modified_base (22)..(22): 2′-fluoro-uridine
  misc_feature (23)..(25): joined through phosphorothioate bonds
  modified_base (23)..(23): 2′-O-methyl-uridine
  modified_base (24)..(24): 2′-O-methyl-uridine
  modified_base (25)..(25): 2′-O-methyl-uridine
uagcaccauuugaaaucaguguuuu

SEQ ID NO: 62 - RNA, 25 nt, Artificial Sequence
  miR-29 mimic antisense/ guide/ first strand
  misc_feature (1)..(3): joined through phosphorothioate bonds
  modified_base (1)..(1): 2′-O-methyl-uridine
  modified_base (2)..(2): 2′-fluoro-adenosine
  modified_base (3)..(3): 2′-O-methyl-guanosine
  modified_base (4)..(4): 2′-fluoro-cytidine
  modified_base (5)..(5): 2′-O-methyl-adenosine
  modified_base (6)..(6): 2′-fluoro-cytidine
  modified_base (7)..(7): 2′-O-methyl-cytidine
  modified_base (8)..(8): 2′-fluoro-adenosine
  modified_base (9)..(9): 2′-O-methyl-uridine
  modified_base (10)..(10): 2′-fluoro-uridine
  modified_base (11)..(11): 2′-O-methyl-uridine
  modified_base (12)..(12): 2′-fluoro-guanosine
  modified_base (13)..(13): 2′-O-methyl-adenosine
  modified_base (14)..(14): 2′-fluoro-adenosine
  modified_base (15)..(15): 2′-O-methyl-adenosine
  modified_base (16)..(16): 2′-O-methyl-uridine
  modified_base (17)..(17): 2′-O-methyl-cytidine
  modified_base (18)..(18): 2′-fluoro-adenosine
  modified_base (19)..(19): 2′-O-methyl-guanosine
  modified_base (20)..(20): 2′-fluoro-uridine
  modified_base (21)..(21): 2′-O-methyl-guanosine
  modified_base (22)..(22): 2′-fluoro-uridine
  misc_feature (23)..(25): joined through phosphorothioate bonds
  modified_base (23)..(23): 2′-O-methyl-uridine
  modified_base (24)..(24): 2′-O-methyl-uridine
  modified_base (25)..(25): 2′-O-methyl-uridine
uagcaccauuugaaaucaguguuuu

SEQ ID NO: 63 - RNA, 25 nt, Artificial Sequence
  miR-29 mimic antisense/ guide/ first strand
  misc_feature (1)..(3): joined through phosphorothioate bonds
  modified_base (1)..(1): 2′-O-methyl-uridine
  modified_base (2)..(2): deoxyadenosine
  modified_base (3)..(3): 2′-O-methyl-guanosine
  modified_base (4)..(4): 2′-fluoro-cytidine
  modified_base (5)..(5): 2′-O-methyl-adenosine
  modified_base (6)..(6): 2′-fluoro-cytidine
  modified_base (7)..(7): 2′-O-methyl-cytidine
  modified_base (8)..(8): deoxyadenosine
  modified_base (9)..(9): 2′-O-methyl-uridine
  modified_base (10)..(10): 2′-fluoro-uridine
  modified_base (11)..(11): 2′-O-methyl-uridine
  modified_base (12)..(12): 2′-O-methyl-guanosine
  modified_base (13)..(13): 2′-O-methyl-adenosine
  modified_base (14)..(14): deoxyadenosine
  modified_base (15)..(15): 2′-O-methyl-adenosine
  modified_base (16)..(16): 2′-fluoro-uridine
  modified_base (17)..(17): 2′-O-methyl-cytidine
  modified_base (18)..(18): deoxyadenosine
  modified_base (19)..(19): 2′-O-methyl-guanosine
  modified_base (20)..(20): 2′-fluoro-uridine
  modified_base (21)..(21): 2′-O-methyl-guanosine
  modified_base (22)..(22): 2′-fluoro-uridine
  misc_feature (23)..(25): joined through phosphorothioate bonds
  modified_base (23)..(23): 2′-O-methyl-uridine
  modified_base (24)..(24): 2′-O-methyl-uridine
  modified_base (25)..(25): 2′-O-methyl-uridine
uagcaccauuugaaaucaguguuuu

SEQ ID NO: 64 - RNA, 25 nt, Artificial Sequence
  miR-29 mimic antisense/ guide/ first strand
  misc_feature (1)..(3): joined through phosphorothioate bonds
  modified_base (1)..(1): 2′-O-methyl-uridine
  modified_base (2)..(2): 2′-O-methyl-adenosine
  modified_base (3)..(3): deoxyguanosine
  modified_base (4)..(4): 2′-O-methyl-cytidine
  modified_base (5)..(5): deoxyadenosine
  modified_base (6)..(6): 2′-O-methyl-cytidine
  modified_base (7)..(7): 2′-fluoro-cytidine
  modified_base (8)..(8): 2′-O-methyl-adenosine
  modified_base (9)..(9): 2′-fluoro-uridine
  modified_base (10)..(10): deoxythymidine
  modified_base (11)..(11): deoxythymidine
  modified_base (12)..(12): deoxyguanosine
  modified_base (13)..(13): deoxyadenosine
  modified_base (14)..(14): 2′-O-methyl-adenosine
  modified_base (15)..(15): deoxyadenosine
  modified_base (16)..(16): 2′-O-methyl-uridine
  modified_base (17)..(17): 2′-fluoro-cytidine
  modified_base (18)..(18): 2′-O-methyl-adenosine
  modified_base (19)..(19): deoxyguanosine
  modified_base (20)..(20): 2′-O-methyl-uridine
  modified_base (21)..(21): deoxyguanosine
  modified_base (22)..(22): 2′-O-methyl-uridine
  misc_feature (23)..(25): joined through phosphorothioate bonds
  modified_base (23)..(23): 2′-fluoro-uridine
  modified_base (24)..(24): 2′-O-methyl-uridine
  modified_base (25)..(25): 2′-O-methyl-uridine
uagcaccaunngaaaucaguguuuu

SEQ ID NO: 65 - RNA, 25 nt, Artificial Sequence
  miR-29 mimic antisense/ guide/ first strand
  misc_feature (1)..(3): joined through phosphorothioate bonds
  modified_base (1)..(1): 2′-O-methyl-uridine
  modified_base (2)..(2): 2′-O-methyl-adenosine
  modified_base (3)..(3): deoxyguanosine
  modified_base (4)..(4): 2′-O-methyl-cytidine
  modified_base (5)..(5): deoxyadenosine
  modified_base (6)..(6): 2′-O-methyl-cytidine
  modified_base (7)..(7): 2′-fluoro-cytidine
  modified_base (8)..(8): 2′-O-methyl-adenosine
  modified_base (9)..(9): 2′-fluoro-uridine
  misc_feature (10)..(13): joined through phosphorothioate bonds
  modified_base (10)..(10): deoxythymidine
  modified_base (11)..(11): deoxythymidine
  modified_base (12)..(12): deoxyguanosine
  modified_base (13)..(13): deoxyadenosine
  modified_base (14)..(14): 2′-O-methyl-adenosine
  modified_base (15)..(15): deoxyadenosine
  modified_base (16)..(16): 2′-O-methyl-uridine
  modified_base (17)..(17): 2′-fluoro-cytidine
  modified_base (18)..(18): 2′-O-methyl-adenosine
  modified_base (19)..(19): deoxyguanosine
  modified_base (20)..(20): 2′-O-methyl-uridine
  modified_base (21)..(21): deoxyguanosine
  modified_base (22)..(22): 2′-O-methyl-uridine
  misc_feature (23)..(25): joined through phosphorothioate bonds
  modified_base (23)..(23): 2′-fluoro-uridine
  modified_base (24)..(24): 2′-O-methyl-uridine
  modified_base (25)..(25): 2′-O-methyl-uridine
uagcaccaunngaaaucaguguuuu

SEQ ID NO: 66 - RNA, 23 nt, Artificial Sequence
  synthetic microRNA-29b mimic sense/ second/ passenger strand
  misc_feature (1)..(1): BiPPB bicyclic peptide conjugate linked through a C6 diene linker
  modified_base (1)..(1): 2′-O-methyl-adenosine
  modified_base (2)..(2): 2′-O-methyl-adenosine
  modified_base (3)..(3): 2′-O-methyl-cytidine
  modified_base (5)..(5): 2′-O-methyl-cytidine
  modified_base (6)..(6): 2′-O-methyl-uridine
  modified_base (8)..(8): 2′-O-methyl-uridine
  modified_base (9)..(9): 2′-O-methyl-uridine
  modified_base (10)..(10): 2′-O-methyl-uridine
  modified_base (12)..(12): 2′-O-methyl-cytidine
  modified_base (16)..(16): 2′-O-methyl-uridine
  modified_base (19)..(19): 2′-O-methyl-uridine
  modified_base (20)..(20): 2′-O-methyl-cytidine
  modified_base (21)..(21): 2′-O-methyl-cytidine
  modified_base (22)..(22): 2′-O-methyl-uridine
aacacuguuuacaaaugguccua

SEQ ID NO: 67 - RNA, 24 nt, Artificial Sequence
  synthetic microRNA-29b mimic sense/ second/ passenger strand
  modified_base (1)..(1): 2′-O-methyl-adenosine
  modified_base (2)..(2): 2′-O-methyl-adenosine
  modified_base (3)..(3): 2′-O-methyl-cytidine
  modified_base (5)..(5): 2′-O-methyl-cytidine
  modified_base (6)..(6): 2′-O-methyl-uridine
  modified_base (8)..(8): 2′-O-methyl-uridine
  modified_base (9)..(9): 2′-O-methyl-uridine
  modified_base (10)..(10): 2′-O-methyl-uridine
  modified_base (12)..(12): 2′-O-methyl-cytidine
  modified_base (16)..(16): 2′-O-methyl-uridine
  modified_base (19)..(19): 2′-O-methyl-uridine
  modified_base (20)..(20): 2′-O-methyl-cytidine
  modified_base (21)..(21): 2′-O-methyl-cytidine
  modified_base (22)..(22): 2′-O-methyl-uridine
  misc_feature (1)..(1): BiPPB bicyclic peptide conjugate linked through a C6 diene linker via phosphorothioate bond
aacacuguuuacaaaugguccuac

SEQ ID NO: 68 - RNA, 23 nt, Artificial Sequence
  synthetic microRNA-29b mimic sense/ second/ passenger strand
  misc_feature (1)..(3): joined through phosphorothioate bonds
  misc_feature (1)..(1): BiPPB bicyclic peptide conjugate linked through a C6 diene linker
  modified_base (1)..(1): 2′-fluoro-adenosine
  modified_base (2)..(2): 2′-O-methyl-adenosine
  modified_base (3)..(3): 2′-fluoro-cytidine
  modified_base (4)..(4): 2′-O-methyl-adenosine
  modified_base (5)..(5): 2′-fluoro-cytidine
  modified_base (6)..(6): 2′-O-methyl-uridine
  modified_base (7)..(7): 2′-fluoro-guanosine
  modified_base (8)..(8): 2′-O-methyl-uridine
  modified_base (9)..(9): 2′-fluoro-uridine
  modified_base (10)..(10): 2′-O-methyl-uridine
  modified_base (11)..(11): 2′-fluoro-adenosine
  modified_base (12)..(12): 2′-fluoro-cytidine
  modified_base (13)..(13): 2′-fluoro-adenosine
  modified_base (14)..(14): 2′-O-methyl-adenosine
  modified_base (15)..(15): 2′-fluoro-adenosine
  modified_base (16)..(16): 2′-O-methyl-uridine
  modified_base (17)..(17): 2′-fluoro-guanosine
  modified_base (18)..(18): 2′-O-methyl-guanosine
  modified_base (19)..(19): 2′-fluoro-uridine
  modified_base (20)..(20): 2′-O-methyl-cytidine
  modified_base (21)..(21): 2′-fluoro-cytidine
  modified_base (22)..(22): 2′-O-methyl-uridine
  modified_base (23)..(23): 2′-fluoro-adenosine
aacacuguuuacaaaugguccua

SEQ ID NO: 69 - RNA, 23 nt, Artificial Sequence
  synthetic microRNA-29b mimic sense/ second/ passenger strand
  misc_feature (1)..(3): joined through phosphorothioate bonds
  misc_feature (1)..(1): BiPPB bicyclic peptide conjugate linked through a C6 diene linker
  modified_base (1)..(1): 2′-fluoro-adenosine
  modified_base (2)..(2): 2′-O-methyl-adenosine
  modified_base (3)..(3): 2′-fluoro-cytidine
  modified_base (4)..(4): 2′-O-methyl-adenosine
  modified_base (5)..(5): 2′-fluoro-cytidine
  modified_base (6)..(6): 2′-O-methyl-uridine
  modified_base (7)..(7): 2′-fluoro-guanosine
  modified_base (8)..(8): 2′-O-methyl-uridine
  modified_base (9)..(9): 2′-fluoro-uridine
  modified_base (10)..(10): 2′-O-methyl-uridine
  modified_base (11)..(11): 2′-fluoro-adenosine
  modified_base (12)..(12): 2′-fluoro-cytidine
  modified_base (13)..(13): 2′-fluoro-adenosine
  modified_base (14)..(14): 2′-O-methyl-adenosine
  modified_base (15)..(15): 2′-fluoro-adenosine
  modified_base (16)..(16): 2′-O-methyl-uridine
  modified_base (17)..(17): 2′-fluoro-guanosine
  modified_base (18)..(18): 2′-O-methyl-guanosine
  modified_base (19)..(19): 2′-fluoro-uridine
  modified_base (20)..(20): 2′-O-methyl-cytidine
  modified_base (21)..(21): 2′-fluoro-cytidine
  modified_base (22)..(22): 2′-O-methyl-uridine
  modified_base (23)..(23): 2′-fluoro-adenosine
aacacuguuuacaaaugguccua

SEQ ID NO: 70 - RNA, 19 nt, Artificial Sequence
  synthetic microRNA-29b mimic sense/ second/ passenger strand
  misc_feature (1)..(3): joined through phosphorothioate bonds
  misc_feature (1)..(1): BiPPB bicyclic peptide conjugate linked through a C6 diene linker
  modified_base (1)..(1): 2′-fluoro-adenosine
  modified_base (2)..(2): 2′-O-methyl-uridine
  modified_base (3)..(3): 2′-fluoro-guanosine
  modified_base (4)..(4): 2′-O-methyl-uridine
  modified_base (5)..(5): 2′-fluoro-adenosine
  modified_base (6)..(6): 2′-O-methyl-cytidine
  modified_base (7)..(7): 2′-fluoro-cytidine
  modified_base (8)..(8): 2′-O-methyl-uridine
  modified_base (9)..(9): 2′-fluoro-guanosine
  modified_base (10)..(10): 2′-O-methyl-adenosine
  modified_base (11)..(11): 2′-fluoro-adenosine
  modified_base (12)..(12): 2′-O-methyl-uridine
  modified_base (13)..(13): 2′-fluoro-uridine
  modified_base (14)..(14): 2′-O-methyl-guanosine
  modified_base (15)..(15): 2′-fluoro-cytidine
  modified_base (16)..(16): 2′-O-methyl-adenosine
  modified_base (17)..(17): 2′-fluoro-cytidine
  modified_base (18)..(18): 2′-O-methyl-adenosine
  modified_base (19)..(19): 2′-fluoro-adenosine
auguaccugaauugcacaa

SEQ ID NO: 71 - RNA, 24 nt, Artificial Sequence
  synthetic microRNA-29b mimic sense/ second/ passenger strand
  misc_feature (1)..(3): joined through phosphorothioate bonds
  modified_base (1)..(1): 2′-fluoro-adenosine
  modified_base (2)..(2): 2′-O-methyl-adenosine
  modified_base (3)..(3): 2′-fluoro-cytidine
  modified_base (4)..(4): 2′-O-methyl-adenosine
  modified_base (5)..(5): 2′-fluoro-cytidine
  modified_base (6)..(6): 2′-O-methyl-uridine
  modified_base (7)..(7): 2′-fluoro-guanosine
  modified_base (8)..(8): 2′-O-methyl-uridine
  modified_base (9)..(9): 2′-fluoro-uridine
  modified_base (10)..(10): 2′-O-methyl-uridine
  modified_base (11)..(11): 2′-fluoro-adenosine
  modified_base (12)..(12): 2′-fluoro-cytidine
  modified_base (13)..(13): 2′-fluoro-adenosine
  modified_base (14)..(14): 2′-O-methyl-adenosine
  modified_base (15)..(15): 2′-fluoro-adenosine
  modified_base (16)..(16): 2′-O-methyl-uridine
  modified_base (17)..(17): 2′-fluoro-guanosine
  modified_base (18)..(18): 2′-O-methyl-guanosine
  modified_base (19)..(19): 2′-fluoro-uridine
  modified_base (20)..(20): 2′-O-methyl-cytidine
  modified_base (21)..(21): 2′-fluoro-cytidine
  modified_base (22)..(22): 2′-O-methyl-uridine
  modified_base (23)..(23): 2′-fluoro-adenosine
  misc_feature (23)..(23): BiPPB biclycic peptide conjugate linked through a C7 diene linker via a phosphorothioate bond
aacacuguuuacaaaugguccuac

SEQ ID NO: 72 - RNA, 23 nt, Artificial Sequence
  synthetic microRNA-29b mimic sense/ second/ passenger strand
  misc_feature (1)..(3): joined through phosphorothioate bonds
  modified_base (1)..(1): 2′-fluoro-adenosine
  modified_base (2)..(2): 2′-O-methyl-adenosine
  modified_base (3)..(3): 2′-fluoro-cytidine
  modified_base (4)..(4): 2′-O-methyl-adenosine
  modified_base (5)..(5): 2′-fluoro-cytidine
  modified_base (6)..(6): 2′-O-methyl-uridine
  modified_base (7)..(7): 2′-fluoro-guanosine
  modified_base (8)..(8): 2′-O-methyl-uridine
  modified_base (9)..(9): 2′-fluoro-uridine
  modified_base (10)..(10): 2′-O-methyl-uridine
  modified_base (11)..(11): 2′-fluoro-adenosine
  modified_base (12)..(12): 2′-fluoro-cytidine
  modified_base (13)..(13): 2′-fluoro-adenosine
  modified_base (14)..(14): 2′-O-methyl-adenosine
  modified_base (15)..(15): 2′-fluoro-adenosine
  modified_base (16)..(16): 2′-O-methyl-uridine
  modified_base (17)..(17): 2′-fluoro-guanosine
  modified_base (18)..(18): 2′-O-methyl-guanosine
  modified_base (19)..(19): 2′-fluoro-uridine
  modified_base (20)..(20): 2′-O-methyl-cytidine
  modified_base (21)..(21): 2′-fluoro-cytidine
  modified_base (22)..(22): 2′-O-methyl-uridine
  modified_base (23)..(23): 2′-fluoro-adenosine
  misc_feature (23)..(23): Retioic acid conjugated via a C3 amino linker via a phosphorothioate bond
aacacuguuuacaaaugguccua

SEQ ID NO: 73 - RNA, 23 nt, Artificial Sequence
  synthetic microRNA-29b mimic sense/ second/ passenger strand
  misc_feature (1)..(3): joined through phosphorothioate bonds
  modified_base (1)..(1): 2′-fluoro-adenosine
  modified_base (2)..(2): 2′-O-methyl-adenosine
  modified_base (3)..(3): 2′-fluoro-cytidine
  modified_base (4)..(4): 2′-O-methyl-adenosine
  modified_base (5)..(5): 2′-fluoro-cytidine
  modified_base (6)..(6): 2′-O-methyl-uridine
  modified_base (7)..(7): 2′-fluoro-guanosine
  modified_base (8)..(8): 2′-O-methyl-uridine
  modified_base (9)..(9): 2′-fluoro-uridine
  modified_base (10)..(10): 2′-O-methyl-uridine
  modified_base (11)..(11): 2′-fluoro-adenosine
  modified_base (12)..(12): 2′-fluoro-cytidine
  modified_base (13)..(13): 2′-fluoro-adenosine
  modified_base (14)..(14): 2′-O-methyl-adenosine
  modified_base (15)..(15): 2′-fluoro-adenosine
  modified_base (16)..(16): 2′-O-methyl-uridine
  modified_base (17)..(17): 2′-fluoro-guanosine
  modified_base (18)..(18): 2′-O-methyl-guanosine
  modified_base (19)..(19): 2′-fluoro-uridine
  modified_base (20)..(20): 2′-O-methyl-cytidine
  modified_base (21)..(21): 2′-fluoro-cytidine
  modified_base (22)..(22): 2′-O-methyl-uridine
  modified_base (23)..(23): 2′-fluoro-adenosine
  misc_feature (23)..(23): Docosanoic Acid conjugated via a phosphorothioate bond
aacacuguuuacaaaugguccua

SEQ ID NO: 74 - RNA, 25 nt, Artificial Sequence
  synthetic microRNA-29b Antisense/ first/ guide strand
  misc_feature (1)..(1): 5′-terminal monophosphate
  modified_base (1)..(1): 2′-fluoro-uridine
  modified_base (4)..(4): 2′-fluoro-cytidine
  modified_base (6)..(6): 2′-fluoro-cytidine
  modified_base (7)..(7): 2′-fluoro-cytidine
  modified_base (9)..(9): 2′-fluoro-uridine
  modified_base (10)..(10): 2′-fluoro-uridine
  modified_base (11)..(11): 2′-fluoro-uridine
  modified_base (16)..(16): 2′-fluoro-uridine
  modified_base (17)..(17): 2′-fluoro-cytidine
  modified_base (20)..(20): 2′-fluoro-uridine
  modified_base (22)..(22): 2′-fluoro-uridine
  misc_feature (23)..(25): joined through phosphorothioate bonds
  modified_base (23)..(23): 2′-fluoro-uridine
uagcaccauuugaaaucaguguuuu

SEQ ID NO: 75 - RNA, 25 nt, Artificial Sequence
  synthetic microRNA-29b Antisense/ first/ guide strand
  misc_feature (1)..(3): joined through phosphorothioate bonds
  misc_feature (1)..(1): 5′-terminal monophosphate
  modified_base (1)..(1): 2′-O-methyl-uridine
  modified_base (2)..(2): 2′-fluoro-adenosine
  modified_base (3)..(3): 2′-O-methyl-guanosine
  modified_base (4)..(4): 2′-fluoro-cytidine
  modified_base (5)..(5): 2′-O-methyl-adenosine
  modified_base (6)..(6): 2′-fluoro-cytidine
  modified_base (7)..(7): 2′-O-methyl-cytidine
  modified_base (8)..(8): 2′-fluoro-adenosine
  modified_base (9)..(9): 2′-O-methyl-uridine
  modified_base (10)..(10): 2′-fluoro-uridine
  modified_base (11)..(11): 2′-O-methyl-uridine
  modified_base (12)..(12): 2′-O-methyl-guanosine
  modified_base (13)..(13): 2′-O-methyl-adenosine
  modified_base (14)..(14): 2′-fluoro-adenosine
  modified_base (15)..(15): 2′-O-methyl-adenosine
  modified_base (16)..(16): 2′-fluoro-uridine
  modified_base (17)..(17): 2′-O-methyl-cytidine
  modified_base (18)..(18): 2′-fluoro-adenosine
  modified_base (19)..(19): 2′-O-methyl-guanosine
  modified_base (20)..(20): 2′-fluoro-uridine
  modified_base (21)..(21): 2′-O-methyl-guanosine
  modified_base (22)..(22): 2′-fluoro-uridine
  misc_feature (23)..(25): joined through phosphorothioate bonds
  modified_base (23)..(23): 2′-O-methyl-uridine
  modified_base (24)..(24): 2′-O-methyl-uridine
  modified_base (25)..(25): 2′-O-methyl-uridine
uagcaccauuugaaaucaguguuuu

SEQ ID NO: 76 - RNA, 21 nt, Artificial Sequence
  synthetic microRNA-29b Antisense/ first/ guide strand
  misc_feature (1)..(3): joined through phosphorothioate bonds
  misc_feature (1)..(1): 5′-terminal monophosphate
  modified_base (1)..(1): 2′-O-methyl-uridine
  modified_base (2)..(2): 2′-fluoro-uridine
  modified_base (3)..(3): 2′-O-methyl-guanosine
  modified_base (4)..(4): 2′-fluoro-adenosine
  modified_base (5)..(5): 2′-O-methyl-guanosine
  modified_base (6)..(6): 2′-fluoro-cytidine
  modified_base (7)..(7): 2′-O-methyl-adenosine
  modified_base (8)..(8): 2′-fluoro-adenosine
  modified_base (9)..(9): 2′-O-methyl-uridine
  modified_base (10)..(10): 2′-fluoro-uridine
  modified_base (11)..(11): 2′-O-methyl-cytidine
  modified_base (12)..(12): 2′-fluoro-adenosine
  modified_base (13)..(13): 2′-O-methyl-cytidine
  modified_base (14)..(14): 2′-fluoro-guanosine
  modified_base (15)..(15): 2′-O-methyl-uridine
  modified_base (16)..(16): 2′-fluoro-uridine
  modified_base (17)..(17): 2′-O-methyl-cytidine
  modified_base (18)..(18): 2′-fluoro-adenosine
  misc_feature (19)..(21): joined through phosphorothioate bonds
  modified_base (19)..(19): 2′-O-methyl-uridine
  modified_base (20)..(20): 2′-O-methyl-uridine
  modified_base (21)..(21): 2′-O-methyl-uridine
uugagcaauucacguucauuu

SEQ ID NO: 77 - RNA, 25 nt, Artificial Sequence
  synthetic microRNA-29b Antisense/ first/ guide strand
  modified_base (1)..(1): 2′-fluoro-uridine
  modified_base (4)..(4): 2′-fluoro-cytidine
  modified_base (6)..(6): 2′-fluoro-cytidine
  modified_base (7)..(7): 2′-fluoro-cytidine
  modified_base (9)..(9): 2′-fluoro-uridine
  modified_base (10)..(10): 2′-fluoro-uridine
  modified_base (11)..(11): 2′-fluoro-uridine
  modified_base (16)..(16): 2′-fluoro-uridine
  modified_base (17)..(17): 2′-fluoro-cytidine
  modified_base (20)..(20): 2′-fluoro-uridine
  modified_base (22)..(22): 2′-fluoro-uridine
  misc_feature (23)..(25): joined through phosphorothioate bonds
  modified_base (23)..(23): 2′-fluoro-uridine
uagcaccauuugaaaucaguguuuu

SEQ ID NO: 78 - protein, 6 aa, Artificial Sequence
  peptide agent
  MISC_FEATURE (3)..(3): Xaa is Asn or Arg
SRXLID

SEQ ID NO: 79 - protein, 8 aa, Artificial Sequence
  peptide agent
  misc_feature (1)..(1): Xaa can be any naturally occurring amino acid capable of forming a peptide bond
  misc_feature (4)..(4): Xaa is Asn or Arg
  misc_feature (8)..(8): Xaa can be any naturally occurring amino acid capable of forming a peptide bond
XSRXLIDX

SEQ ID NO: 80 - protein, 20 aa, Artificial Sequence
  linker
  misc_feature (2)..(20): May be present or up to 19 may be absent
GGGGGGGGGGGGGGGGGGGG

SEQ ID NO: 81 - protein, 40 aa, Artificial Sequence
  linker
  misc_feature (3)..(4): May be present or absent
  misc_feature (5)..(6): May be present or absent
  misc_feature (7)..(8): May be present or absent
  misc_feature (9)..(10): May be present or absent
  misc_feature (11)..(12): May be present or absent
  misc_feature (13)..(14): May be present or absent
  misc_feature (15)..(16): May be present or absent
  misc_feature (17)..(18): May be present or absent
  misc_feature (19)..(20): May be present or absent
  misc_feature (21)..(22): May be present or absent
  misc_feature (23)..(24): May be present or absent
  misc_feature (25)..(26): May be present or absent
  misc_feature (27)..(28): May be present or absent
  misc_feature (29)..(30): May be present or absent
  misc_feature (31)..(32): May be present or absent
  misc_feature (33)..(34): May be present or absent
  misc_feature (35)..(36): May be present or absent
  misc_feature (37)..(38): May be present or absent
  misc_feature (39)..(40): May be present or absent
GSGSGSGSGSGSGSGSGSGSGSGSGSGSGSGSGSGSGSGS

SEQ ID NO: 82 - protein, 100 aa, Artificial Sequence
  linker
  misc_feature (6)..(10): May be present or absent
  misc_feature (11)..(15): May be present or absent
  misc_feature (16)..(20): May be present or absent
  misc_feature (21)..(25): May be present or absent
  misc_feature (26)..(30): May be present or absent
  misc_feature (31)..(35): May be present or absent
  misc_feature (36)..(40): May be present or absent
  misc_feature (41)..(45): May be present or absent
  misc_feature (46)..(50): May be present or absent
  misc_feature (51)..(55): May be present or absent
  misc_feature (56)..(60): May be present or absent
  misc_feature (61)..(65): May be present or absent
  misc_feature (66)..(70): May be present or absent
  misc_feature (71)..(75): May be present or absent
  misc_feature (76)..(80): May be present or absent
  misc_feature (81)..(85): May be present or absent
  misc_feature (86)..(90): May be present or absent
  misc_feature (91)..(95): May be present or absent
  misc_feature (96)..(100): May be present or absent
GSGGSGSGGSGSGGSGSGGSGSGGSGSGGSGSGGSGSGGSGSGGSGSGGSGSGGSGSGGSGSGGSGSGGSGSGGSGSGGSGSGGSGSGGSGSGGSGSGGS

SEQ ID NO: 83 - protein, 80 aa, Artificial Sequence
  linker
  misc_feature (5)..(8): May be present or absent
  misc_feature (9)..(12): May be present or absent
  misc_feature (13)..(16): May be present or absent
  misc_feature (17)..(20): May be present or absent
  misc_feature (21)..(24): May be present or absent
  misc_feature (25)..(28): May be present or absent
  misc_feature (29)..(32): May be present or absent
  misc_feature (33)..(36): May be present or absent
  misc_feature (37)..(40): May be present or absent
  misc_feature (41)..(44): May be present or absent
  misc_feature (45)..(48): May be present or absent
  misc_feature (49)..(52): May be present or absent
  misc_feature (53)..(56): May be present or absent
  misc_feature (57)..(60): May be present or absent
  misc_feature (61)..(64): May be present or absent
  misc_feature (65)..(68): May be present or absent
  misc_feature (69)..(72): May be present or absent
  misc_feature (73)..(76): May be present or absent
  misc_feature (77)..(80): May be present or absent
GGGSGGGSGGGSGGGSGGGSGGGSGGGSGGGSGGGSGGGSGGGSGGGSGGGSGGGSGGGSGGGSGGGSGGGSGGGSGGGS

SEQ ID NO: 84 - protein, 22 aa, Artificial Sequence
  PDGF-R binding bicyclic peptide
  DISULFID (1)..(8): Cys residues joined by disulfide bridge
  DISULFID (15)..(22): Cys residues joined by disulfide bridge
CSRNLIDCGGGDGGCSRNLIDC

SEQ ID NO: 85 - protein, 8 aa, Artificial Sequence
  cyclic peptide
  DISULFID (1)..(8): Cys residues joined by disulfide bridge
CSRNLIDC

SEQ ID NO: 86 - protein, 22 aa, Artificial Sequence
  bicyclic peptide
  DISULFID (1)..(8): Cys residues joined by disulfide bridge
  DISULFID (15)..(22): Cys residues joined by disulfide bridge
CSRRLIDCGGGDGGCSRRLIDC

SEQ ID NO: 87 - protein, 17 aa, Artificial Sequence
  bicyclic peptide
  misc_feature (9)..(9): Xaa is a linker of any amino acid, -(-CH2-)-, -(-CH2CH2O-)-, -[-NH-(CH2)y-NH-]- or nucleotide
CSRNLIDCXCSRNLIDC

SEQ ID NO: 88 - protein, 17 aa, Artificial Sequence
  bicyclic peptide
  misc_feature (9)..(9): Xaa is a linker of any amino acid, -(-CH2-)-, -(-CH2CH2O-)-, -[-NH-(CH2)y-NH-]- or nucleotide
CSRRLIDCXCSRRLIDC

SEQ ID NO: 89 - protein, 22 aa, Artificial Sequence
  linear peptide that can be conjugated to polynucleotide
CSRNLIDCGGGDGGCSRNLIDC

SEQ ID NO: 90 - protein, 18 aa, Artificial Sequence
  linear peptide that can be conjugated to polynucleotide
SRNLIDGGGDGGSRNLID

SEQ ID NO: 91 - protein, 22 aa, Artificial Sequence
  linear peptide that can be conjugated to polynucleotide
CSRRLIDCGGGDGGCSRRLIDC

SEQ ID NO: 92 - protein, 18 aa, Artificial Sequence
  linear peptide that can be conjugated to polynucleotide
SRRLIDGGGDGGSRRLID

SEQ ID NO: 93 - protein, 8 aa, Artificial Sequence
  linear peptide that can be conjugated to polynucleotide
CSRNLIDC

SEQ ID NO: 94 - protein, 6 aa, Artificial Sequence
  linear peptide that can be conjugated to polynucleotide
SRNLID

SEQ ID NO: 95 - protein, 7 aa, Artificial Sequence
  linear peptide that can be conjugated to polynucleotide
SRRLIDC

SEQ ID NO: 96 - protein, 6 aa, Artificial Sequence
  linear peptide that can be conjugated to polynucleotide
SRRLID

SEQ ID NO: 97 - RNA, 23 nt, Artificial Sequence
  synthetic microRNA-29b mimic sense/ second/ passenger strand
  misc_feature (1)..(3): joined through phosphorothioate bonds
  misc_feature (1)..(1): Human BiPPB biclycic peptide conjugate linked through a C7 diene linker and phosphorothioate
  modified_base (1)..(1): 2′-fluoro-adenosine
  modified_base (2)..(2): 2′-O-methyl-adenosine
  modified_base (3)..(3): 2′-fluoro-cytidine
  modified_base (4)..(4): 2′-O-methyl-adenosine
  modified_base (5)..(5): 2′-fluoro-cytidine
  modified_base (6)..(6): 2′-O-methyl-uridine
  modified_base (7)..(7): 2′-fluoro-guanosine
  modified_base (8)..(8): 2′-O-methyl-uridine
  modified_base (9)..(9): 2′-fluoro-uridine
  modified_base (10)..(10): 2′-O-methyl-uridine
  modified_base (11)..(11): 2′-fluoro-adenosine
  modified_base (12)..(12): 2′-fluoro-cytidine
  modified_base (13)..(13): 2′-fluoro-adenosine
  modified_base (14)..(14): 2′-O-methyl-adenosine
  modified_base (15)..(15): 2′-fluoro-adenosine
  modified_base (16)..(16): 2′-O-methyl-uridine
  modified_base (17)..(17): 2′-fluoro-guanosine
  modified_base (18)..(18): 2′-O-methyl-guanosine
  modified_base (19)..(19): 2′-fluoro-uridine
  modified_base (20)..(20): 2′-O-methyl-cytidine
  modified_base (21)..(21): 2′-fluoro-cytidine
  modified_base (22)..(22): 2′-O-methyl-uridine
  modified_base (23)..(23): 2′-fluoro-adenosine
aacacuguuuacaaaugguccua

SEQ ID NO: 98 - RNA, 23 nt, Artificial Sequence
  synthetic microRNA-29b mimic sense/ second/ passenger strand
  misc_feature (1)..(3): joined through phosphorothioate bonds
  misc_feature (1)..(1): Mouse BiPPB biclycic peptide conjugate linked through a C7 diene linker and phosphorothioate
  modified_base (1)..(1): 2′-fluoro-adenosine
  modified_base (2)..(2): 2′-O-methyl-adenosine
  modified_base (3)..(3): 2′-fluoro-cytidine
  modified_base (4)..(4): 2′-O-methyl-adenosine
  modified_base (5)..(5): 2′-fluoro-cytidine
  modified_base (6)..(6): 2′-O-methyl-uridine
  modified_base (7)..(7): 2′-fluoro-guanosine
  modified_base (8)..(8): 2′-O-methyl-uridine
  modified_base (9)..(9): 2′-fluoro-uridine
  modified_base (10)..(10): 2′-O-methyl-uridine
  modified_base (11)..(11): 2′-fluoro-adenosine
  modified_base (12)..(12): 2′-fluoro-cytidine
  modified_base (13)..(13): 2′-fluoro-adenosine
  modified_base (14)..(14): 2′-O-methyl-adenosine
  modified_base (15)..(15): 2′-fluoro-adenosine
  modified_base (16)..(16): 2′-O-methyl-uridine
  modified_base (17)..(17): 2′-fluoro-guanosine
  modified_base (18)..(18): 2′-O-methyl-guanosine
  modified_base (19)..(19): 2′-fluoro-uridine
  modified_base (20)..(20): 2′-O-methyl-cytidine
  modified_base (21)..(21): 2′-fluoro-cytidine
  modified_base (22)..(22): 2′-O-methyl-uridine
  modified_base (23)..(23): 2′-fluoro-adenosine
aacacuguuuacaaaugguccua

(19) United States
(12) Patent Application Publication
(10) Pub. No.: US 2020/0318113 A1
(43) Pub. Date: Oct. 8, 2020

(54) POLYNUCLEOTIDE CONJUGATES AND USES THEREOF

(71) Applicant: MIRAGEN THERAPEUTICS, INC., Boulder, CO (US)

(72) Inventors: Subhadeep Roy, Lafayette, CO (US); Kurt Vagle, Boulder, CO (US); Kevin Rigby, Boulder, CO (US); Rusty L. Montgomery, Boulder, CO (US)

(21) Appl. No.: 16/810,771

(22) Filed: Mar. 5, 2020

Related U.S. Application Data
(60) Provisional application No. 62/814,222, filed on Mar. 5, 2019.

(51) Int. Cl. C12N 15/113; A61K 47/62; A61K 47/54; A61P 37/06

(52) U.S. Cl. CPC C12N 15/113; A61K 47/62; A61K 47/542; A61K 47/543; C12N 2310/3513; A61K 47/549; C12N 2310/351; C12N 2310/3515; C12N 2310/141; A61P 37/06

(57) ABSTRACT

Provided herein are polynucleotide conjugates comprising a polynucleotide and an agent, wherein the polynucleotide comprises a synthetic double stranded miR-29 mimic and the agent facilitates the delivery of the polynucleotide to a cell type or tissue type involved in fibrosis or inflammation. Also provided are methods of making, and uses thereof.

### CROSS REFERENCE TO RELATED APPLICATIONS

This application claims the benefit of priority to U.S. Provisional Patent Application No. 62/814,222, filed on Mar. 5, 2019, the content of which is hereby incorporated by reference in its entirety for all purposes.

### BACKGROUND

MicroRNAs (miRNAs) are naturally occurring, short RNA molecules that as part of a ribonucleoprotein complex known as RISC (RNA-induced silencing complex), act to repress translation of specific mRNA molecules. Levels of miRNAs have been demonstrated to be depressed below their normal levels in a variety of diseased states. Therefore a potential mode of therapy in these indications is the introduction of synthetic miRNAs to mimic the activity of natural miRNAs (miRNA mimics). However synthetic RNA molecules that are chemically identical to naturally occurring RNAs are not effective drugs due to their poor stability towards nucleases present in serum. Thus a variety of chemical modifications of the ribose sugar moiety and the phosphate linking group have been developed to confer nuclease stability on synthetic polynucleotides. Also needed are targeting agents to achieve desired biological effects. Therefore, there remains a need for developing optimally modified and targeted double-stranded RNA molecules that act as effective microRNA mimics. Provided herein are compositions and methods that address this need.

### SUMMARY

The present disclosure provides polynucleotide conjugates comprising a polynucleotide and an agent, wherein the polynucleotide comprises a synthetic double stranded miR-29 mimic and the agent facilitates the delivery of the polynucleotide to a cell type or tissue type. In some embodiments, the cell type or tissue type is involved in fibrosis or inflammation. In some embodiments, the agent is a fibrotic pathway agent. Also provided are pharmaceutical compositions thereof, as well as methods of making and methods of use thereof.

Accordingly in one aspect, provided herein are polynucleotide conjugates comprising a polynucleotide and an agent, wherein the polynucleotide comprises a double stranded miR-29 mimic, each strand comprising a plurality of nucleotide modifications, and wherein the agent facilitates the delivery of the polynucleotide to a cell type or tissue type involved in fibrosis or inflammation.

In another aspect, provided herein are polynucleotide conjugates comprising a polynucleotide and an agent, wherein the polynucleotide comprises a double stranded miR-29 mimic, each strand comprising a plurality of nucleotide modifications, and wherein the agent comprises a fibrotic pathway agent.

In some embodiments of the polynucleotide conjugates, the agent comprises a fibrotic pathway agent. In some embodiments, the agent is selected from a peptide, a small molecule, and a fatty acid. In some embodiments, the fibrotic pathway agent is a fibrotic pathway targeting agent. In some embodiments, the fibrotic pathway agent is a fibrotic pathway activating agent. In some embodiments, the fibrotic pathway agent is a fibrotic pathway inhibiting agent. In some embodiments, the agent is a peptide.

In some embodiments of the polynucleotide conjugates, the agent is a peptide, and is a cyclic peptide. In some embodiments, the peptide comprises at least one copy of the amino acid sequence SRZLID, wherein Z is either an asparagine (N) or an arginine (R). In some embodiments, the cyclic peptide is a bicyclic peptide. In some embodiments, the cyclic peptide comprises the amino acid sequence X1SRZLIDX2-linker-X3SRZLIDX4, wherein Z is either an asparagine (N) or an arginine (R), wherein the pair of X1 and X2, and the pair of X3 and X4 can form a peptidic bond to form a bicyclic structure, and wherein the sequences SRZLID are each part of a ring, and optionally wherein the linker is of size n, wherein n is 1-20. In some embodiments, the peptide is a receptor-binding peptide. In some embodiments, the peptide is a PDGF receptor-binding peptide. In some embodiments, the PDGF receptor-binding peptide is derived from PDGF-beta.

In some embodiments of the polynucleotide conjugates, the fibrotic pathway agent is a small molecule. In some embodiments, the small molecule is a retinoid. In some embodiments, the retinoid is an all trans retinoid. In some embodiments, the retinoid is retinoic acid. In some embodiments, the fibrotic pathway agent is a fatty acid. In some embodiments, the fatty acid is a long chain fatty acid. In some embodiments, the long fatty acid comprises 8-30 carbons. In some embodiments, the long fatty acid is a long-chain dicarboxylic acid. In some embodiments, the long fatty acid is docosanoic acid.

In some embodiments, the miR29 mimics comprise a first strand comprising a mature miR-29a, miR-29b, or miR-29c sequence, and a second strand comprising a sequence that is substantially complementary to the first strand. In some embodiments, the first strand comprises a mature miR-29a. In some embodiments, the first strand comprises a mature miR-29b. In some embodiments, the first strand comprises a mature miR-29c. In some embodiments, the agent is conjugated to the first strand. In some embodiments, the agent is conjugated to the 5′-end of the first strand. In some embodiments, the agent is conjugated to the 3′-end of the first strand. In some embodiments, the agent is conjugated to the second strand. In some embodiments, the agent is conjugated to the 5′-end of the second strand. In some embodiments, the agent is conjugated to the 3′-end of the second strand. In some embodiments, the agent is conjugated to an internal position of the second strand. In some embodiments, the agent is conjugated to the miR-29 mimic via a linker. In some embodiments, the agent is conjugated to the miR-29 mimic via cholesterol.

In some embodiments, each nucleotide of the first strand is modified. In some embodiments, each nucleotide of the second strand is modified. In some embodiments, at least 50% of the nucleotides in the first strand are modified. In some embodiments, at least 50% of the nucleotides in the second strand are modified. In some embodiments, the nucleotides of at least 50% of the first and at least 50% of the second strand are modified. In some embodiments, each nucleotide of the first strand, and each nucleotide of the second strand is modified. In some embodiments, the first strand comprises a polynucleotide sequence selected from SEQ ID Nos. 6-9, 23-32, 38-41, 55-65 and 74-77. In some embodiments, the second strand comprises a polynucleotide sequence selected from SEQ ID Nos. 1-5, 10-22, 33-37, 42-54, 66-73, and 97-98. In some embodiments, the first strand comprises a polynucleotide sequence selected from SEQ ID Nos. 74-77 and 55. In some embodiments, the second strand comprises a polynucleotide sequence selected from SEQ ID Nos. 66-73, 97, 98, 45, 53 and 54. In some embodiments, the second strand comprises a polynucleotide sequence of SEQ ID NO: 68, and the first strand comprises a polynucleotide sequence of SEQ ID NO: 75. In some embodiments, the second strand comprises a polynucleotide sequence of SEQ ID NO: 69, and the first strand comprises a polynucleotide sequence of SEQ ID NO: 55. In some embodiments, the second strand comprises a polynucleotide sequence of SEQ ID NO: 71, and the first strand comprises a polynucleotide sequence of SEQ ID NO: 55. In some embodiments, the second strand comprises a polynucleotide sequence of SEQ ID NO: 72, and the first strand comprises a polynucleotide sequence of SEQ ID NO: 55. In some embodiments, the second strand comprises a polynucleotide sequence of SEQ ID NO: 97, and the first strand comprises a polynucleotide sequence of SEQ ID NO: 55. In some embodiments, the second strand comprises a polynucleotide sequence of SEQ ID NO: 98, and the first strand comprises a polynucleotide sequence of SEQ ID NO: 55. In some embodiments, the second strand comprises a polynucleotide sequence of SEQ ID NO: 53, and the first strand comprises a polynucleotide sequence of SEQ ID NO: 55. In some embodiments, the second strand comprises a polynucleotide sequence of SEQ ID NO: 42, and the first strand comprises a polynucleotide sequence of SEQ ID NO: 60. In some embodiments, the second strand comprises a polynucleotide sequence of SEQ ID NO: 43, and the first strand comprises a polynucleotide sequence of SEQ ID NO: 55. In some embodiments, the second strand comprises a polynucleotide sequence of SEQ ID NO: 44, and the first strand comprises a polynucleotide sequence of SEQ ID NO: 60. In some embodiments, the second strand comprises a polynucleotide sequence of SEQ ID NO: 45, and the first strand comprises a polynucleotide sequence selected from SEQ ID NOs: 55-57. In some embodiments, the second strand comprises a polynucleotide sequence of SEQ ID NO: 46, and the first strand comprises a polynucleotide sequence of SEQ ID NO: 55. In some embodiments, the second strand comprises a polynucleotide sequence of SEQ ID NO: 47, and the first strand comprises a polynucleotide sequence of SEQ ID NO: 61. In some embodiments, the second strand comprises a polynucleotide sequence of SEQ ID NO: 48, and the first strand comprises a polynucleotide sequence of SEQ ID NO: 62.

In another aspect, provided herein are pharmaceutical compositions comprising one or more of the polynucleotide conjugates.

In another aspect, provided herein are methods of treating fibrosis or a fibrosis-related condition comprising administering to a subject in need a therapeutically effective amount of one or more of the polynucleotide conjugates or the pharmaceutical compositions thereof.

### BRIEF DESCRIPTION OF THE DRAWINGS

FIG. 1 shows the scheme for the synthesis of C6 diene phosphoramidite and C6 diene derivatized controlled pore glass (CPG).

FIG. 2 shows the scheme for the synthesis of C7 diene derivatized controlled pore glass.

FIG. 3 shows the synthesis scheme for a double stranded polynucleotide, such as a miR-29b mimic, conjugated to BiPPB.

FIG. 4 shows the synthesis scheme for a double stranded polynucleotide, such as a miR-29b mimic, conjugated to retinoic acid.

FIGS. 5A-5G show the results of in vivo evaluation of BiPPB-conjugated miR-29b mimics in idiopathic pulmonary fibrosis (IPF) mouse model. FIG. 5A shows the schedule of administration of bleomycin and miR-29 mimics. FIG. 5B shows a graph comparing the expression of miR-29 target genes on day 14 post-bleomycin in mice which were administered different miR-29b mimics as indicated, normalized to a control group which was treated with only saline, post-bleomycin. FIG. 5C-G show graphs comparing the levels of different miR-29b mimics in lung (FIG. 5C); liver (FIG. 5D); kidney (FIG. 5E); spleen (FIG. 5F); and heart (FIG. 5G).

FIG. 6A shows the dosage schedule for bleomycin and three different dosage schedules for the administration of miR-29 mimic. FIGS. 6B and 6C show graphs comparing the expression of pro-fibrotic genes in mice after administration of either Mimic #8 or Mimic #4 using a day 14 dosage schedule (FIG. 6B) or day 21 dosage schedule (FIG. 6C), normalized to the expression of pro-fibrotic genes in control (bleomycin/saline treated) animals.

FIGS. 7A-7C show the results from real-time PCR of miR-29b from lung (FIG. 7A); kidney (FIG. 7B) and liver (FIG. 7C) tissues.

FIGS. 8A-8D show the lack of liver and kidney toxicity after administration of miR-29b mimics as measured by levels of alanine transaminase (FIG. 8A); aspartate transaminase (FIG. 8B); blood urea nitrogen (FIG. 8C); and creatinine (FIG. 8D).

FIG. 9 shows representative images of Haemotoxylin and Eosin stained; and trichome stained sections of lungs from animals treated with bleomycin and saline; or bleomycin and Mimic #4 using a day 14 dosage schedule.

FIG. 10 shows representative images of Haemotoxylin and Eosin stained; and trichrome stained sections of lungs from animals treated with bleomycin and saline; or bleomycin and Mimic #4 using a day 21 dosage schedule.

FIG. 11 shows the results from a quantitative analysis of trichrome-stained lung sections from animals treated with the indicated compounds at the indicated time-points.

FIGS. 12A-12B show the results of in vivo evaluation of BiPPB-conjugated and retinoic acid-conjugated miR-29b mimics in liver fibrosis mouse model. FIG. 12A shows the schedule of administration of CCl₄ and miR-29 mimics. FIG. 12B shows a graph comparing the expression of miR-29 target genes in mice which were administered with different miR-29b mimics, as compared with control mice which were administered CCl₄ and saline.

FIG. 13 shows a graph comparing the levels of different miR-29b mimics in kidney; liver; lung; and spleen.

FIG. 14 shows potential alternate configurations of PDGFβ-R targeting peptide-polynucleotide conjugates.

FIG. 15 shows the structure of bicyclic peptide derived from the human PDGF-β protein sequence. An Arg residue is present in the human sequence (next to Leu) when compared to the mouse sequence that contains an Asn residue at that position.

FIGS. 16A-16B show designs for double stranded polynucleotides conjugated with bis-monocyclic peptides in FIG. 16A (Design I) and FIG. 16B (Design II).

FIG. 17 shows the synthesis scheme for generating a double stranded polynucleotide conjugated with bis-monocyclic peptide of Design I.

FIG. 18 shows the synthesis scheme for generating a double stranded polynucleotide conjugated with bis-monocyclic peptide of Design II.

FIG. 19 shows structures of generalized linkers connecting cyclic peptides.

FIG. 20 shows structures of linear peptides that can be conjugated to polynucleotides.

FIG. 21 shows a schematic representation of methods to conjugate generalized linkers with polynucleotides.

FIG. 22 shows a scheme for the synthesis of docosanoic acid (DCA) conjugated miR-29b mimic.

FIG. 23 shows a scheme for the synthesis of a BiPPB conjugated to a double strand miR-29 mimic polynucleotide.

FIG. 24A shows a configuration of PDGFβ-R targeting BiPPB with a linker. FIG. 24B shows a configuration of PDGFβ-R targeting BiPPB conjugated to a polynucleotide.

FIG. 25 shows a fully drawn out configuration of PDGFβ-R targeting BiPPB.

FIG. 26A-26B shows the results of in vitro cell studies for evaluating the efficacy of human or mouse PDGF BiPPB conjugated miR-29 mimics to down-regulate Col1a1 expression.

FIG. 26A shows the result of mouse embryonic fibroblasts (MEFs). FIG. 26B shows the result of a human lung epithelial cell line (A549).

FIG. 27 shows the results of an ex vivo study for evaluating the efficacy of human or mouse PDGF BiPPB conjugated miR-29 mimics to regulate collagen level using human precision-cut lung slices.

FIG. 28 shows the dosage schedule of bleomycin and BiPPB conjugated miR-29 mimic for an in vivo study.

FIG. 29 shows the results of an in vivo study for evaluating the efficacy of human or mouse PDGF BiPPB conjugated miR-29 in bleomycin-induced fibrotic mice.

### DETAILED DESCRIPTION

The present disclosure provides polynucleotide conjugates comprising a polynucleotide and an agent, wherein the polynucleotide comprises a synthetic double stranded miR-29 mimic and the agent facilitates the delivery of the polynucleotide to a cell type or tissue type. In some embodiments, the cell type or tissue type is involved in fibrosis or inflammation. In some embodiments, the agent is a fibrotic pathway agent. Also provided are pharmaceutical compositions thereof, as well as methods of making and methods of use thereof.

### I. Double Stranded MicroRNA-29 Mimics

A microRNA mimic according to the disclosure comprises a first strand and a second strand, wherein the first strand comprises a mature miR-29a, miR-29b, or miR-29c sequence and the second strand comprises a sequence that is substantially complementary to the first strand and has at a plurality of modified nucleotides. Throughout the disclosure, the term “microRNA mimic” may be used interchangeably with the terms “promiR-29,” “miR-29 agonist,” “microRNA agonist,” “microRNA mimic,” “miRNA mimic,” or “miR-29 mimic;” the term “first strand” may be used interchangeably with the terms “antisense strand” or “guide strand”; the term “second strand” may be used interchangeably with the term “sense strand” or “passenger strand;” and the term “miR-29 antagonist” may be used interchangeably with the terms “polynucleotide inhibitor,” “antimiR-29,” “antisense polynucleotide,” “miR-29 antagomir” or “anti-microRNA polynucleotide.”

In some embodiments, the first strand of the microRNA mimic comprises from about 19 to about 28 nucleotides comprising a sequence of mature miR-29a, miR-29b, or miR-29c and the second strand comprises from about 19 to about 28 nucleotides comprising a sequence that is partially, substantially, or fully complementary to the first strand. In some embodiments, the first strand of the microRNA mimic comprises from about 23 to about 26 nucleotides comprising a sequence of mature miR-29a, miR-29b, or miR-29c and the second strand comprises from about 22 to about 24 nucleotides comprising a sequence that is partially, substantially, or fully complementary to the first strand. In various embodiments, the first strand may comprise about 23, 24, 25, or 26 nucleotides and the second strand may comprise about 22, 23 or 24 nucleotides.

The nucleotides that form the first and the second strand of the microRNA mimics may comprise ribonucleotides, deoxyribonucleotides, modified nucleotides, and combinations thereof. In certain embodiments, the first strand and the second strand of the microRNA mimic comprise ribonucleotides and/or modified ribonucleotides. The term “modified nucleotide” means a nucleotide where the nucleobase and/or the sugar moiety is modified relative to unmodified nucleotides.

In certain embodiments, the microRNA mimics have a first strand or an antisense strand, whose sequence is identical to all or part of a mature miR-29a, miR-29b, or miR-29c sequence, and a second strand or a sense strand whose sequence is about 70% to about 100% complementary to the sequence of the first strand. In some embodiments, the first strand of the miRNA mimic is at least about 75, 80, 85, 90, 95, or 100% identical, including all integers there between, to the entire sequence of a mature, naturally occurring miR-29a, miR-29b, or miR-29c sequence. In certain embodiments, the first strand is about or is at least about 90, 91, 92, 93, 94, 95, 96, 97, 98, 99, or 100% identical to the sequence of a mature, naturally-occurring miRNA, such as the mouse, human, or rat miR-29a, miR-29b, or miR-29c sequence. Alternatively, the first strand may comprise 20, 21, 22, or 23 nucleotide positions in common with a mature, naturally-occurring miRNA as compared by sequence alignment algorithms and methods well known in the art.

It is understood that the sequence of the first strand is considered to be identical to the sequence of a mature miR-29a, miR-29b, or miR-29c even if the first strand includes a modified nucleotide instead of a naturally-occurring nucleotide. For example, if a mature, naturally-occurring miRNA sequence comprises a cytidine nucleotide at a specific position, the first strand of the mimic may comprise a modified cytidine nucleotide, such as 2′-fluoro-cytidine, at the corresponding position or if a mature, naturally-occurring miRNA sequence comprises a uridine nucleotide at a specific position, the miRNA region of the first strand of the mimic may comprise a modified uridine nucleotide, such as 2′-fluoro-uridine, 2′-O-methyl-uridine, 5-fluorouracil, or 4-thiouracil at the corresponding position. Thus, as long as the modified nucleotide has the same base-pairing capability as the nucleotide present in the mature, naturally-occurring miRNA sequence, the sequence of the first strand is considered to be identical to the mature, naturally-occurring miRNA sequence. In some embodiments, the first strand may include a modification of the 5′-terminal residue. For example, the first strand may have a 5′-terminal monophosphate. In some other embodiments, the first strand does not contain a 5′-terminal monophosphate.

In some embodiments, the second strand of the microRNA mimic is partially complementary to the sequence of the first strand. For example, the sequence of the second strand is at least about 70%, 75%, 80%, 85%, 90%, 95%, 96%, 97%, 98%, or 99%, inclusive of all values therebetween, complementary to the sequence of the first strand. In some other embodiments, the second strand is substantially complementary to the sequence of the first strand. For example, the second strand is at least about 85%, 86%, 87%, 88%, 89%, 90%, 91%, 92%, 93%, 94%, 95%, 96%, 97%, 98%, or 99%, inclusive of all values therebetween, complementary to the sequence of the first strand. In yet some other embodiments, the sequence of the second strand may be fully complementary to the first strand. In certain embodiments, about 19, 20, 21, 22, or 23 nucleotides of the complementary region of the second strand may be complementary to the first strand.

It is understood that the sequence of the second strand is considered to be complementary to the first strand even if the second strand includes a modified nucleotide instead of a naturally-occurring nucleotide. For example, if the first strand sequence comprises a guanosine nucleotide at a specific position, the second strand may comprise a modified cytidine nucleotide, such as 2′-O-methyl-cytidine, at the corresponding position.

In some embodiments, the second strand comprises about 1, 2, 3, 4, 5, or 6 mismatches relative to the first strand. That is, up to 1, 2, 3, 4, 5, or 6 nucleotides between the first strand and the second strand may not be complementary. In some embodiments, the mismatches are not consecutive and are distributed throughout the second strand. In another embodiment, the mismatches are consecutive and may create a bulge. In some embodiments, the second strand contains 3 mismatches relative to the first strand. In certain embodiments, the second strand of a miR-29a mimic or a miR-29c mimic contains mismatches at positions 4, 13, and/or 16 from the 3′ end (of the second strand) relative to the first strand. In some embodiments, the second strand of a miR-29b mimic contains mismatches at positions 4, 13, and/or 16 from the 3′ end (of the second strand) relative to the first strand. In another embodiment, the second strand of a miR-29b mimic contains mismatches at positions 4, 9, 10, 11, 13 and/or 16 from the 3′ end (of the second strand) relative to the first strand.

In some embodiments, the first and/or the second strand of the mimic may comprise an overhang on the 5′ or 3′ end of the strands. In certain embodiments, the first strand comprises a 3′ overhang, i.e., a single-stranded region that extends beyond the duplex region, relative to the second strand. The 3′ overhang of the first strand may range from about one nucleotide to about four nucleotides. In certain embodiments, the 3′ overhang of the first strand may comprise 1 or 2 nucleotides. In some embodiments, the nucleotides comprising the 3′ overhang in the first strand are linked by phosphorothioate linkages. The nucleotides comprising the 3′ overhang in the first strand may include ribonucleotides, deoxyribonucleotides, modified nucleotides, or combinations thereof. In certain embodiments, the 3′ overhang in the first strand comprises two ribonucleotides. In some embodiments, the 3′ overhang of the first strand comprises two uridine nucleotides linked through a phosphorothioate linkage. In some embodiments, the first strand may not contain an overhang.

In some embodiments, the nucleotides in the second/sense strand of miR-29 mimics of the disclosure are linked by phosphodiester linkages and the nucleotides in the first/antisense strand are linked by phosphodiester linkages. In some embodiments all except for the last set of nucleotides at the 3′ end of the first strand are linked by phosphodiester linkage and the last set of nucleotides are linked to each other via phosphorothioate linkages.

The first and the second strand of microRNA mimics of the disclosure can also include backbone modifications, such as one or more phosphorothioate, phosphorodithioate, phosphotriester, boranophosphate, alkylphosphonates, phosphoramidates, phosphordiamidates, thionophosphoramidates, thionoalkylphosphonates, thionoalkylphosphotriesters, or phosphonocarboxylate linkages, where the linkage is the conventional 3′-5′ linkage, 2′-5′ linked analog or inverted linkages such as 3′-3′, 5′-5′ and 2′-2′.

The first and/or the second strand of microRNA mimics of the disclosure can also include modifications on the sugar residue, such as ribose modification/replacement. For instance, the first and/or the second strand of the microRNA mimics of the disclosure may include one or more morpholinos, peptide nucleic acids, serinol nucleic acids, locked nucleic acids (LNA), and unlocked nucleic acids.

In various embodiments, miR-29 mimics of the present disclosure comprise a plurality of modified nucleotides. For instance, in some embodiments, the first strand and/or the second strand of the mimic comprises two or more 2′-fluoro nucleotides. In some embodiments, the first strand and/or the second strand comprises two or more 2′-O-methyl modified nucleotides. In some embodiments, the first strand and/or the second strand comprises two or more deoxynucleotides.

The modified nucleotides that may be used in the microRNA mimics of the disclosure can include nucleotides with a base modification or substitution. The natural or unmodified bases in RNA are the purine bases adenine (A) and guanine (G), and the pyrimidine bases cytosine (C) and uracil (U) (DNA has thymine (T)). In contrast, modified bases, also referred to as heterocyclic base moieties, include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo (including 5-bromo, 5-trifluoromethyl and other 5-substituted uracils and cytosines), 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine.

In some embodiments, the microRNA mimics can have nucleotides with modified sugar moieties. Representative modified sugars include carbocyclic or acyclic sugars, sugars having substituent groups at one or more of their 2′, 3′ or 4′ positions and sugars having substituents in place of one or more hydrogen atoms of the sugar. In certain embodiments, the sugar is modified by having a substituent group at the 2′ position. In additional embodiments, the sugar is modified by having a substituent group at the 3′ position. In other embodiments, the sugar is modified by having a substituent group at the 4′ position. It is also contemplated that a sugar may have a modification at more than one of those positions, or that an RNA molecule may have one or more nucleotides with a sugar modification at one position and also one or more nucleotides with a sugar modification at a different position.

Sugar modifications contemplated in the miRNA mimics include, but are not limited to, a substituent group selected from: OH; F; O—, S—, or N-alkyl; O—, S—, or N-alkenyl; 0-, S- or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl.

In some embodiments, miRNA mimics have a sugar substituent group selected from the following: C₁ to C₁₀ lower alkyl, substituted lower alkyl, alkenyl, alkynyl, alkaryl, aralkyl, O-alkaryl or O-aralkyl, SH, SCH₃, Cl, Br, CN, OCN, CF₃, OCF₃, SOCH₃, SO₂CH₃, ONO₂, NO₂, N₃, NH₂, heterocycloalkyl, heterocycloalkaryl, aminoalkylamino, or similar substituents. In one embodiment, the modification includes 2′-methoxyethoxy (2′-O—CH₂CH₂OCH₃, which is also known as 2′-O-(2-methoxyethyl) or 2′-MOE), that is, an alkoxyalkoxy group. Another modification includes 2′-dimethylaminooxyethoxy, that is, a O(CH₂)₂ON(CH₃)₂ group, also known as 2′-DMAOE and 2′-dimethylaminoethoxyethoxy (also known in the art as 2′-O-dimethyl-amino-ethoxy-ethyl or 2′-DMAEOE), that is, 2′-O—CH₂—O—CH₂—N(CH₃)₂.

Sugar substituent groups on the 2′ position (2′-) may be in the arabino (up) position or ribo (down) position. One 2′-arabino modification is 2′-F. Other similar modifications may also be made at other positions on the sugar moiety, particularly the 3′ position of the sugar on the 3′ terminal nucleoside or in 2′-5′ linked polynucleotides and the 5′ position of 5′ terminal nucleotide.

In certain embodiments, the sugar modification is a 2′-O-alkyl (e.g. 2′-O-methyl, 2′-O-methoxyethyl), 2′-halo (e.g., 2′-fluoro, 2′-chloro, 2′-bromo), and 4′ thio modifications. For instance, in some embodiments, the first strand of the miR-29a, miR-29b, or miR-29c mimic comprises one or more 2′ fluoro nucleotides. In another embodiment, the first strand of the mimics has no modified nucleotides. In yet another embodiment, the second strand of miR-29a, miR-29b, or miR-29c mimic comprises one or more 2′-O-methyl modified nucleotides.

In some embodiments, at least 50% of the nucleotides in the first strand are modified. In some embodiments, at least 50% of the nucleotides in the second strand are modified. In some embodiments, the nucleotides of at least 50% of the first and at least 50% of the second strand are modified. In some embodiments, each nucleotide of the first strand, and each nucleotide of the second strand is modified.

Table 1 provides a guide to the abbreviations used herein to describe the plurality of modifications present on the miR29 mimics of the disclosure.

Tables 2-6 provide exemplary partially or fully modified miR-29a, miR-29b, and miR-29c mimics of the disclosure, wherein the mimics are conjugated to a fibrotic pathway agent of the disclosure as described in further detail below. As described herein, BPM refers to a mouse BPM or a human BPM.

TABLE 1
Definitions of Abbreviations

| Nucleotide unit or | | Nucleotide unit or | |
|---|---|---|---|
| modification | Abbreviation | modification | Abbreviation |
| ribo A | rA | ribo A P═S | rAs |
| ribo G | rG | ribo G P═S | rGs |
| ribo C | rC | ribo C P═S | rCs |
| ribo U | rU | ribo U P═S | rUs |
| O-methyl A | mA | O-methyl A P═S | mAs |
| O-methyl G | mG | O-methyl G P═S | mGs |
| O-methyl C | mC | O-methyl C P═S | mCs |
| O-methyl U | mU | O-methyl U P═S | mUs |
| fluoro C | fC | fluoro C P═S | fCs |
| fluoro U | fU | fluoro U P═S | fUs |
| deoxy A | dA | deoxy A P═S | dAs |
| deoxy G | dG | deoxy G P═S | dGs |
| deoxy C | dC | deoxy C P═S | dCs |
| deoxy T | dT | deoxy T P═S | dTs |
| monophosphate | p | | |
| Cholesterol | Chol6/C6 | | |
| conjugate with a 6 | chol | | |
| carbon linker | | | |
| Cholesterol | Chol9 | | |
| conjugate with a 9 | | | |
| carbon linker | | | |
| Docosanoic Acid | DCA | | |
| Retioic acid | PTA.RET | | |
| conjugated likned | | | |
| via a C3 amino | | | |
| linker | | | |
| BiPPB bicyclic | BPM.C6D | Mouse PDGF BiPPB | mBPM.C7Ds |
| peptide conjugate | | bicyclic peptide | |
| linked through a | | conjugate linked | |
| C6 diene | | through a C7 diene | |
| linker | | linker P═S | |
| BiPPB bicyclic | C7D-sup.BPM | Human PDFG BiPPB | hBPM.C7Ds |
| peptide conjugate | | bicyclic peptide | |
| linked through a | | conjugate linked | |
| C7 diene linker | | through a C7 diene | |
| | | linker P═S | |
| GalNac conjugate | GalNP5s | GalNac conjugate at | GalNP5 |
| linked via | | 3′ end | |
| phosphorothioate | | | |

TABLE 2
Exemplary miR-29a mimics

| | SEQ |
|---|---|
| Modified Sequence | ID NO: |
| Second/sense/passenger strands | |
| 5′-mU.mA.rA.rC.rC.rG.rA.rU.rU.rU.rC.rA.rG.rA.rU.rG.rG.rU.rG.rC.rU. | 1 |
| rA.rU.rU-3′ | |
| 5′- | 2 |
| mU.mA.rA.mC.mC.rG.mU.mU.mU.rA.mC.rA.rG.rA.mU.rG.rG.mU.mC.mC.mU.rA-3′ | |
| 5′- | 3 |
| mU.mA.rA.mC.mC.rG.mU.mU.mU.rA.mC.rA.rG.rA.mU.rG.rG.mU.mC.mC.mU. | |
| rA.chol6-3′ | |
| 5′- | 4 |
| mU.mA.rA.rC.rC.rG.rA.rU.rU.rU.rC.rA.rG.rA.rU.rG.rG.rU.rG.rC.rU.rAs.rUs. | |
| rUs.chol6-3′ | |
| 5′-mU.mA.rA.rC.rC.rG.rA.rU.rU.rU.rC.rA.rG.rA.rU.rG.rG.rU.rG.rC.rU.rA-3′ | 5 |
| First/antisense/guide strands | |
| 5′-p.rU.rA.rG.rC.rA.rC.rC.rA.rU.rC.rU.rG.rA.rA.rA.rU.rC.rG.rG.rU.rU.rA. | 6 |
| rU.rU-3′ | |
| 5′-p.fU.rA.rG.fC.rA.fC.fC.rA.fU.fC.fU.rG.rA.rA.rA.fU.fC.rG.rG.fU.fU.rAs. | 7 |
| rUs.rU-3′ | |
| 5′-fU.rA.rG.fC.rA.fC.fC.rA.fU.fC.fU.rG.rA.rA.rA.fU.fC.rG.rG.fU.fU.rAs. | 8 |
| rUs.rU-3′ | |
| 5′-rU.rA.rG.rC.rA.rC.rC.rA.rU.rC.rU.rG.rA.rA.rA.rU.rC.rG.rG.rU.rU.rA.rU. | 9 |
| rU-3′ | |

TABLE 3
Exemplary miR-29b mimics

| | SEQ |
|---|---|
| Modified Sequence | ID NO: |
| Second/sense/passenger strands | |
| 5′- | 10 |
| mA.mA.rC.rA.rC.rU.rG.rA.rU.rU.rU.rC.rA.rA.rA.rU.rG.rG.rU.rG.rC.rU.rA.rU.rU- | |
| 3′ | |
| 5′- | 11 |
| mA.mA.mC.rA.mC.mU.rG.rA.mU.mU.mU.mC.rA.rA.rA.mU.rG.rG.mU.rG.mC.mU. | |
| rA.chol6-3′ | |
| 5′- | 12 |
| mA.mA.rC.rA.rC.rU.rG.rA.rU.rU.rU.rC.rA.rA.rA.rU.rG.rG.rU.rG.rC.rU.rAs.rUs.rUs. | |
| chol6-3′ | |
| 5′- | 13 |
| mA.mA.mC.rA.mC.mU.rG.mU.mU.mU.rA.mC.rG.rG.rG.mU.rG.rG.mU.mC.mC.mU. | |
| rA-3′ | |
| 5′- | 14 |
| mA.mA.mC.rA.mC.mU.rG.mU.mU.mU.rA.mC.rG.rG.rG.mU.rG.rG.mU.mC.mC.mU. | |
| rA.chol6-3′ | |
| 5′- | 15 |
| mA.mA.mC.rA.mC.mU.rG.mU.mU.mU.rA.mC.rA.rA.rA.mU.rG.rG.mU.mC.mC.mU. | |
| rA.chol6-3′ | |
| 5′- | 16 |
| mA.mA.mC.rA.mC.mU.rG.mU.mU.mU.rA.mC.rA.rA.rA.mU.rG.rG.mU.mC.mC.mU. | |
| rA.dT.dT.chol6-3′ | |
| 5′- | 17 |
| C6Chol.dT.dT.mA.mA.mC.rA.mC.mU.rG.mU.mU.mU.rA.mC.rA.rA.rA.mU.rG.rG. | |
| mU.mC.mC.mU.rA-3′ | |
| 5′- | 18 |
| mA.mA.mC.rA.mC.mU.rG.mU.mU.mU.rA.mC.rA.rA.rA.mU.rG.rG.mU.mC.mC.mU. | |
| rA.chol9-3′ | |
| 5′- | 19 |
| rA.mA.rC.mA.rC.mU.rG.mA.rU.mU.rU.mC.rA.mA.rA.mU.rG.mG.rU.mG.rC.mU.rA. | |
| chol6-3′ | |
| 5′- | 20 |
| rA.mA.rC.mA.rC.mU.rG.mA.rU.mU.rU.mC.rA.mA.rA.mU.rG.mG.rU.mG.rC.mU.rAs. | |
| rUs.rU.chol6-3′ | |
| 5′-mA.mA.mC.rA.mC.mU.rG.mU.mU.mU.rA.mC.rA.rA.rA.mU.rG.rG.mU.mC. | 21 |
| mC.mU.rA.cholTEG-3′ | |
| 5′-mA.mA.rC.rA.rC.rU.rG.rA.rU.rU.rU.rC.rA.rA.rA.rU.rG.rG.rU.rG.rC.rU.rA-3′ | 22 |
| First/antisense/guidestrands | |
| 5′- | 23 |
| p.rU.rA.rG.rC.rA.rC.rC.rA.rU.rU.rU.rG.rA.rA.rA.rU.rC.rA.rG.rU.rG.rU.rU.rU.rU-3′ | |
| 5′- | 24 |
| p.fU.rA.rG.fC.rA.fC.fC.rA.fU.fU.fU.rG.rA.rA.rA.fU.fC.rA.rG.fU.rG.fU.fUs.rUs.rU- | |
| 3′ | |
| 5′- | 25 |
| fU.rA.rG.fC.rA.fC.fC.rA.fU.fU.fU.rG.rA.rA.rA.fU.fC.rA.rG.fU.rG.fU.fUs.rUs.rU-3′ | |
| 5′- | 26 |
| p.fU.rA.rG.fC.rA.fC.fC.rA.fC.fC.fC.rG.rA.rA.rA.fU.fC.rA.rG.fU.rG.fU.fUs.rUs.rU- | |
| 3′ | |
| 5′-rU.rA.rG.rC.rA.rC.rC.rA.rU.rU.rU.rG.rA.rA.rA.rU.rC.rA.rG.rU.rG.rU.rU.rU.rU-3′ | 27 |
| 5′- | 28 |
| mU.rA.mG.rC.mA.rC.mC.rA.mU.rU.mU.rG.mA.rA.mA.rU.mC.rA.mG.rU.mG.rU.mU-3′ | |
| 5′ | 29 |
| mU.rA.mG.rC.mA.rC.mC.rA.mU.rU.mU.rG.mA.rA.mA.rU.mC.rA.mG.rU.mG.rU.mUs. | |
| rUs.rU-3′ | |
| 5′-mU.rA.rG.mC.rA.mC.mC.rA.mU.mU.mU.rG.rA.rA.rA.mU.mC.rA.rG.mU.rG. | 30 |
| mU.mUs.rUs.rU-3′ | |
| 5′-fU.rA.rG.fC.rA.fC.fC.rA.fU.fU.fU.rG.rA.rA.rA.fU.fC.rA.rG.fU.rG.fU.fU-3′ | 31 |
| 5′-rU.rA.rG.rC.rA.rC.rC.rA.rU.rU.rU.rG.rA.rA.rA.rU.rC.rA.rG.rU.rG.rU.rU.rU.rU- | 32 |
| 3′ | |

TABLE 4
Exemplary miR-29c mimics

| | SEQ |
|---|---|
| Modified Sequence | ID NO: |
| Second/sense/passenger strands | |
| 5′- | 33 |
| mU.mA.rA.rC.rC.rG.rA.rU.rU.rU.rC.rA.rA.rA.rU.rG.rG.rU.rG.rC.rU.rA.rU.rU- | |
| 3′ | |
| 5′- | 34 |
| mU.mA.rA.mC.mC.rG.mU.mU.mU.rA.mC.rA.rA.rA.mU.rG.rG.mU.mC.mC.mU. | |
| rA-3′ | |
| 5′- | 35 |
| mU.mA.rA.mC.mC.rG.mU.mU.mU.rA.mC.rA.rA.rA.mU.rG.rG.mU.mC.mC.mU. | |
| rA.chol6-3′ | |
| 5′- | 36 |
| mU.mA.rA.rC.rC.rG.rA.rU.rU.rU.rC.rA.rA.rA.rU.rG.rG.rU.rG.rC.rU.rAs.rUs. | |
| rUs.chol6-3′ | |
| 5′-mU.mA.rA.rC.rC.rG.rA.rU.rU.rU.rC.rA.rA.rA.rU.rG.rG.rU.rG.rC.rU.rA-3′ | 37 |
| First/antisense/guide strands | |
| 5′- | 38 |
| p.rU.rA.rG.rC.rA.rC.rC.rA.rU.rU.rU.rG.rA.rA.rA.rU.rC.rG.rG.rU.rU.rA.rU. | |
| rU-3′ | |
| 5′- | 39 |
| p.fU.rA.rG.fC.rA.fC.fC.rA.fU.fU.fU.rG.rA.rA.rA.fU.fC.rG.rG.fU.fU.rAs.rUs. | |
| rU-3′ | |
| 5′-fU.rA.rG.fC.rA.fC.fC.rA.fU.fU.fU.rG.rA.rA.rA.fU.fC.rG.rG.fU.fU.rAs.rUs. | 40 |
| rU-3′ | |
| 5′-rU.rA.rG.rC.rA.rC.rC.rA.rU.rU.rU.rG.rA.rA.rA.rU.rC.rG.rG.rU.rU.rA.rU. | 41 |
| rU-3′ | |

TABLE 5
Sense/Passenger/Second strands of exemplary miR-29 mimics, with exemplary duplex
combinations

| | Oligo# | | Duplexed |
|---|---|---|---|
| Description | (SEQ ID) | Sequence | with |
| Fully modified, stretch | SEQ ID NO: 42 | 5′-fAs.mAs.fC.mA.fC.mU.fG.mU. | SEQ ID |
| of 3 consecutive F- | | fU.fU.fA.mC.fA.mA.fA.mU.fG.mG. | NO: 60 |
| modified nucleotides | | fU.mC.fC.mU.fAs.GalNP5s. | |
| | | GalNP5s.GalNP5-3′ | |
| Fully modified, stretch | SEQ ID NO: 43 | 5′-fAs.mAs.fC.mA.fC.mU.fG.mU. | SEQ ID |
| of 3 consecutive F- | | fU.mU.fA.fC.fA.mA.fA.mU.fG.mG. | NO: 55 |
| modified nucleotides | | fU.mC.fC.mU.fAs.GalNP5s. | |
| | | GalNP5s.GalNP5-3′ | |
| Fully modified, stretch | SEQ ID NO: 44 | 5′-fAs.mAs.fC.mA.fC.mU.fG.mU. | SEQ ID |
| of 3 consecutive F- | | fU.fU.fA.mC.fA.mA.fA.mU.fG.mG. | NO: 60 |
| modified nucleotides | | fU.mC.fC.mU.fA-3′ | |
| Fully modified, stretch | SEQ ID NO: 45 | 5′-fAs.mAs.fC.mA.fC.mU.fG.mU. | SEQ ID |
| of 3 consecutive F- | | fU.mU.fA.fC.fA.mA.fA.mU.fG.mG. | NO: 55, |
| modified nucleotides | | fU.mC.fC.mU.fA-3′ | SEQ ID |
| | | | NO: 56, or |
| | | | SEQ ID |
| | | | NO: 57 |
| Fully modified, stretch | SEQ ID NO: 46 | 5′-fAs.mAs.fC.mA.fC.mU.fG.mU. | SEQ ID |
| of 3 consecutive F- | | fU.mU.fA.fC.fA.mA.fA.mU.fG.mG. | NO: 55 |
| modified nucleotides | | fU.mC.fC.mU.fAs.GalNP5s. | |
| | | GalNP5s.GalNP5-3′ | |
| Fully modified, stretch | SEQ ID NO: 47 | 5′-fAs.mAs.fC.mA.fC.mU.fG.mU. | SEQ ID |
| of 3 consecutive F- | | fU.mU.fA.mC.fA.fA.fA.mU.fG.mG. | NO: 61 |
| modified nucleotides | | fU.mC.fC.mU.fA-3′ | |
| Fully modified, stretch | SEQ ID NO: 48 | 5′-fAs.mAs.fC.mA.fC.mU.fG.fU.fU. | SEQ ID |
| of 3 consecutive F- | | mU.fA.mC.fA.fA.fA.mU.fG.mG.fU. | NO: 62 |
| modified nucleotides | | mC.fC.mU.fA-3′ | |
| Contains unmodified | SEQ ID NO: 49 | 5′-dAs.mAs.fC.mA.fC.mU.dG.mU. | SEQ ID |
| deoxy nucleotides | | fU.mU.dA.fC.dA.mA.dA.mU.dG. | NO: 63 |
| | | mG.fU.mC.fC.mUs.dA-3′ | |
| Contains unmodified | SEQ ID NO: 50 | 5′-mAs.dAs.mC.dA.mC.fU.mG.fU. | SEQ ID |
| deoxy nucleotides | | mU.fU.mA.fC.dA.dA.mA.fU.mG. | NO: 64 |
| | | dG.mU.fC.mC.fUs.dA-3′ | |
| Contains unmodified | SEQ ID NO: 51 | 5′-mAs.dAs.mC.dA.mC.fU.mG.fU. | SEQ ID |
| deoxy nucleotides | | mU.fU.mAs.fCs.dAs.dA.mA.fU. | NO: 65 |
| | | mG.dG.mU.fC.mC.fUs.dA-3′ | |
| Fully modified, stretch | SEQ ID NO: 52 | 5′-fAs.mAs.fC.mA.fC.mU.fG.mU. | SEQ ID |
| of 3 consecutive F- | | fU.mU.fA.fC.fA.mA.fA.mU.fG.mG. | NO: 55 |
| modified nucleotides | | fU.mC.fC.mU.fA.chol6-3′ | |
| Fully modified, stretch | SEQ ID NO: 53 | 5′-fAs.mAs.fC.mA.fC.mU.fG.mU. | SEQ ID |
| of 3 consecutive F- | | fU.mU.fA.fC.fA.mA.fA.mU.fG.mG. | NO: 55 |
| modified nucleotides | | fU.mC.fC.mU.fAs.chol6-3′ | |
| Partially modified | SEQ ID NO: 54 | 5′-mA.mA.mC.rA.mC.mU.rG.mU. | SEQ ID |
| | | mU.mU.rA.mC.rA.rA.rA.mU.rG.rG. | NO: 58 |
| | | mU.mC.mC.mU.rA.chol6-3′ | |

TABLE 6
Antisense/Guide/First strands of exemplary miR-29 mimics, with exemplary duplex
combinations

| | Oligo# | | Duplexed |
|---|---|---|---|
| Description | (SEQ ID) | Sequence | with |
| Fully modified | SEQ ID NO: 55 | 5′-mUs.fAs.mG.fC.mA.fC.mC.fA. | SEQ ID |
| | | mU.fU.mU.mG.mA.fA.mA.fU.mC. | NO: 45 |
| | | fA.mG.fU.mG.fU.mUs.mUs.mU-3′ | |
| Fully modified | SEQ ID NO: 56 | 5′-mUs.fAs.mG.lC.mA.fC.mC.fA. | SEQ ID |
| | | mU.fU.mU.mG.mA.fA.mA.fU.mC. | NO: 45 |
| | | fA.mG.fU.mG.fU.mUs.mUs.mU-3′ | |
| Fully modified | SEQ ID NO: 57 | 5′-mUs.fAs.mG.lC.mA.fC.mC.fA. | SEQ ID |
| | | mU.fU.mU.mG.mA.fA.mA.lT.mC. | NO: 45 |
| | | fA.mG.fU.mG.fU.mUs.mUs.mU-3′ | |
| Partially modified | SEQ ID NO: 58 | 5′-p.fU.rA.rG.fC.rA.fC.fC.rA.fU.fU. | SEQ ID |
| | | fU.rG.rA.rA.rA.fU.fC.rA.rG.fU.rG. | NO: 54 |
| | | fU.fUs.rUs.rUs.Fluor4-3′ | |
| Fully modified | SEQ ID NO: 59 | 5′-mUs.fAs.mG.fC.mA.fC.mC.fA. | SEQ ID |
| | | mU.fU.mU.mG.mA.fA.mA.fU.mC. | NO: 53 |
| | | fA.mG.fU.mG.fU.mUs.mUs.mUs. | |
| | | Fluor4-3′ | |
| Fully Modified | SEQ ID NO: 60 | 5′-mUs.fAs.mG.fC.mA.fC.mC.fA. | SEQ ID |
| | | mU.fU.mU.fG.mA.mA.mA.fU.mC. | NO: 44 |
| | | fA.mG.fU.mG.fU.mUs.mUs.mU-3′ | |
| Fully modified | SEQ ID NO: 61 | 5′-mUs.fAs.mG.fC.mA.fC.mC.fA. | SEQ ID |
| | | mU.mU.mU.fG.mA.fA.mA.fU.mC. | NO: 47 |
| | | fA.mG.fU.mG.fU.mUs.mUs.mU-3′ | |
| Fully modified | SEQ ID NO: 62 | 5′-mUs.fAs.mG.fC.mA.fC.mC.fA. | SEQ ID |
| | | mU.fU.mU.fG.mA.fA.mA.mU.mC. | NO: 48 |
| | | fA.mG.fU.mG.fU.mUs.mUs.mU-3′ | |
| Contains unmodified | SEQ ID NO: 63 | 5′-mUs.dAs.mG.fC.mA.fC.mC.dA. | SEQ ID |
| deoxy nucleotides | | mU.fU.mU.mG.mA.dA.mA.fU.mC. | NO: 49 |
| | | dA.mG.fU.mG.fU.mUs.mUs.mU-3′ | |
| Contains unmodified | SEQ ID NO: 64 | 5′-mUs.mAs.dG.mC.dA.mC.fC.mA. | SEQ ID |
| deoxy nucleotides | | fU.dT.dT.dG.dA.mA.dA.mU.fC. | NO: 50 |
| | | mA.dG.mU.dG.mU.fUs.mUs.mU-3′ | |
| Contains unmodified | SEQ ID NO: 65 | 5′-mUs.mAs.dG.mC.dA.mC.fC.mA. | SEQ ID |
| deoxy nucleotides | | fU.dTs.dTs.dGs.dA.mA.dA.mU.fC. | NO: 51 |
| | | mA.dG.mU.dG.mU.fUs.mUs.mU-3′ | |

### II. Agents

As provided herein, the polynucleotides of the disclosure are conjugated to an agent, wherein the agent facilitates the delivery of the polynucleotide to a cell type or tissue type. In some embodiments, the cell type or tissue type is involved in fibrosis or inflammation.

In some embodiments, the miR-29 mimics of the disclosure are conjugated to a fibrotic pathway agent.

As used herein, a “fibrotic pathway agent” refers to an agent that binds to a target in a fibrosis-related signal transduction pathway. A fibrotic pathway agent can be any type of molecule, for example a peptide, a small molecule, a fatty acid, or a polynucleotide. In some embodiments, the fibrotic pathway agent is a fibrotic pathway targeting agent, in that it binds to a target involved in a fibrosis-related signal transduction pathway, but does not necessarily exert any effect upon binding, e.g. does not necessarily transduce intracellular signal transduction effect upon binding. In some embodiments, the fibrotic pathway agent is a fibrotic pathway activating agent, in that it binds a target involved in a fibrosis-related signal transduction pathway, and sets off/activates signal transduction through the target. In some embodiments, the fibrotic pathway agent is a fibrotic pathway inhibiting agent, in that it binds a target involved in a fibrosis-related signal transduction pathway, and inhibits signal transduction through the target.

In some embodiments, the miR-29 mimics that are conjugated to the fibrotic pathway agent have a sense strand comprising or consisting of SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, or SEQ ID NO: 54. In exemplary embodiments, a miR-29 mimic that is conjugated to the fibrotic pathway agent has a sense strand comprising or consisting of SEQ ID NO: 42. In exemplary embodiments, a miR-29 mimic that is conjugated to the fibrotic pathway agent has a sense strand comprising or consisting of SEQ ID NO: 44. In exemplary embodiments, a miR-29 mimic that is conjugated to the fibrotic pathway agent has a sense strand comprising or consisting of SEQ ID NO: 45. In exemplary embodiments, a miR-29 mimic that is conjugated to the fibrotic pathway agent has a sense strand comprising or consisting of SEQ ID NO: 50.

In some embodiments, the agent is a peptide. In some embodiments, the peptide is a cyclic peptide. In some embodiments, the peptide is a monocyclic peptide. In some embodiments, the peptide is a bicyclic peptide.

In some embodiments, the agent is a peptide, and the peptide comprises one at least one copy of the amino acid sequence SRZLID, wherein Z is either an asparagine (N) or an arginine (R). In some embodiments, the peptide is monocyclic, in some embodiments, the peptide in bicyclic. In some embodiments, the miR-29 mimics that are conjugated to the monocyclic or bicyclic SRZLID peptide, wherein Z is either an asparagine (N) or arginine (R), have a sense strand comprising or consisting of SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, or SEQ ID NO: 54. In exemplary embodiments, a miR-29 mimic that is conjugated to the monocyclic or bicyclic SRZLID peptide, wherein Z is either an asparagine (N) or arginine (R), has a sense strand comprising or consisting of SEQ ID NO: 42. In exemplary embodiments, a miR-29 mimic that is conjugated to the monocyclic or bicyclic SRZLID peptide, wherein Z is either an asparagine (N) or arginine (R), has a sense strand comprising or consisting of SEQ ID NO: 44. In exemplary embodiments, a miR-29 mimic that is conjugated to the monocyclic or bicyclic SRZLID peptide, wherein Z is either an asparagine (N) or arginine (R), has a sense strand comprising or consisting of SEQ ID NO: 45. In exemplary embodiments, a miR-29 mimic that is conjugated to the monocyclic or bicyclic SRZLID peptide, wherein Z is either an asparagine (N) or arginine (R), has a sense strand comprising or consisting of SEQ ID NO: 50.

In some embodiments, the agent is a peptide, and the peptide comprises the amino acid sequence X1SRZLIDX2-linker-X3SRZLIDX4, wherein Z is either an asparagine (N) or an arginine (R), wherein the pair of X1 and X2, and the pair of X3 and X4 can form a bond (e.g. a peptidic bond), such that a bicyclic structure is formed, and wherein the sequences SRZLID are each part of a ring. In some embodiments, the linker of size n, wherein n is 1-20.

In some embodiments, the agent comprises a bicyclic peptide derived from the human PDGF-β protein sequence. In some embodiments, the peptide comprises the amino acid sequence X1SRRLIDX2-linker-X3SRRLIDX4, wherein the pair of X1 and X2, and the pair of X3 and X4 can form a bond (e.g. a peptidic bond), such that a bicyclic structure is formed, and wherein the sequences SRRLID are each part of a ring. In some embodiments, X1, X2, X3 and X4 are cysteine (C). In some embodiments, the bonds formed between the pair of X1 and X2 and between the pair of X3 and X4 are disulfide bonds.

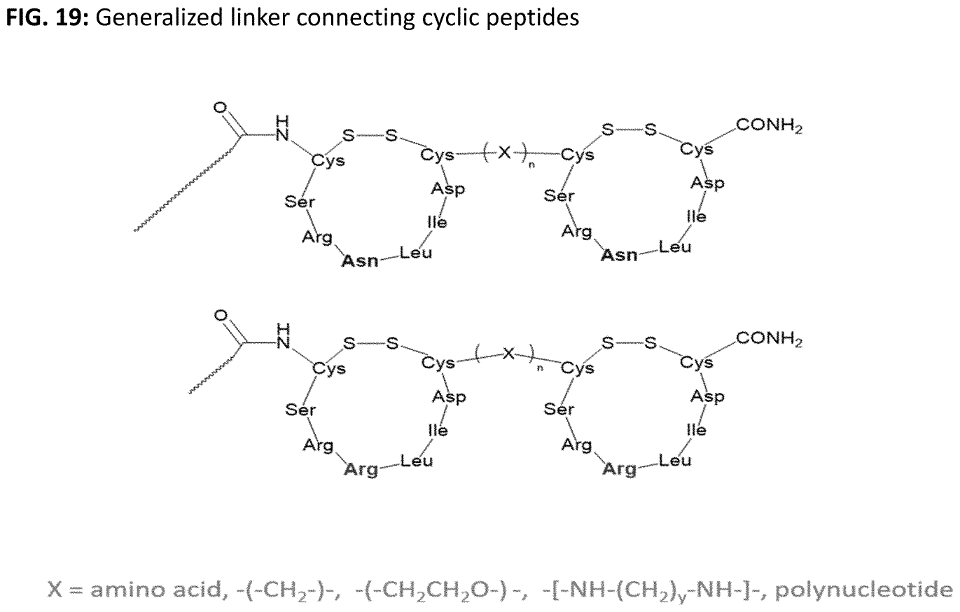

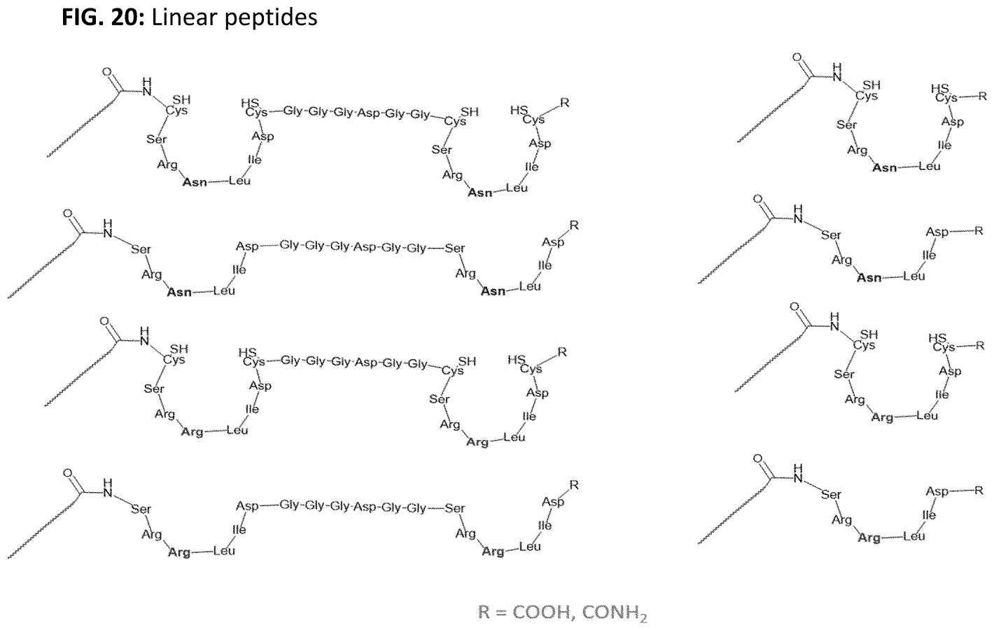

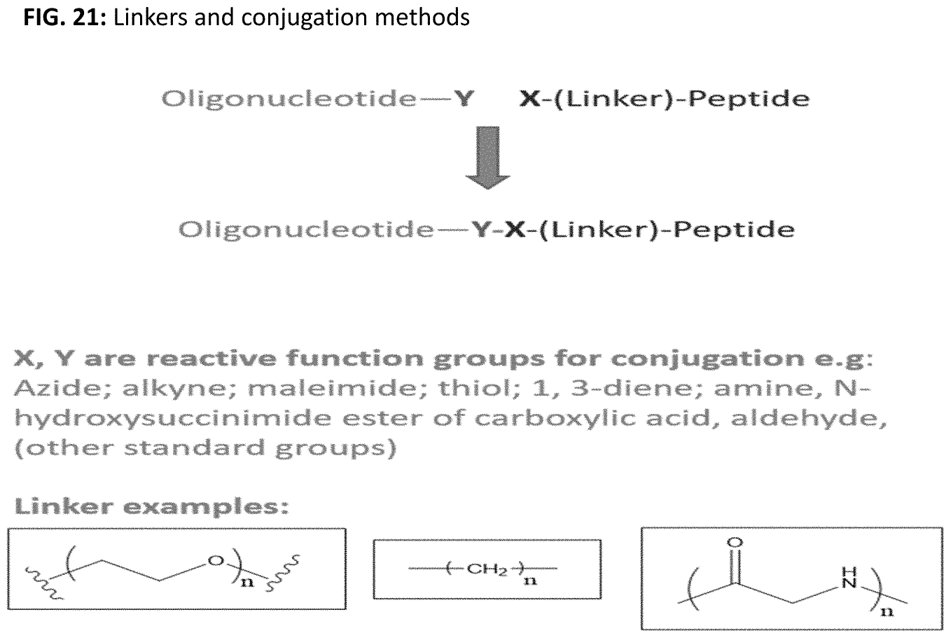

The middle linker in the bicyclic peptide moiety can be any linker as described in the present disclosure, including but not limited to those listed in FIG. 19. In some embodiments, the middle linker is of size n, wherein n is 1-20. In some embodiments, the middle linker is a peptide with amino acid sequence GGGDGG. In some embodiments, the middle linker is conjugated to a cysteine in each of the cyclic peptide moiety.

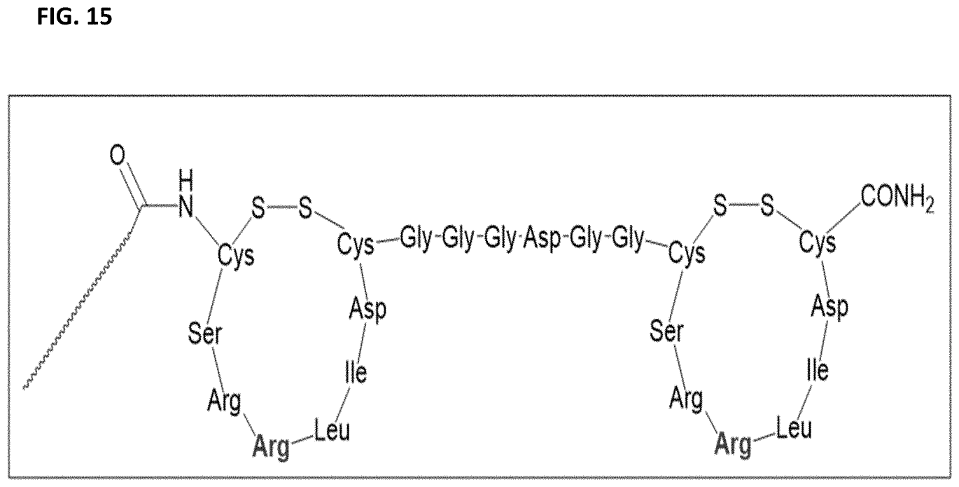

In some embodiments, the bicyclic peptide moiety is conjugated to the rest of the agent by forming an amide bond via the amine group on the backbone. In some embodiments, the bicyclic peptide moiety is conjugated to the rest of the agent via a functional group in the middle linker. In some embodiments, the carboxylate end of the bicyclic peptide is capped by formation of an amide (e.g., —C(O)NH2). In some embodiments, the bicyclic peptide moiety and its conjugation to the rest of the compound are the same as illustrated in FIG. 15.

In some embodiments, the agent is connected to the polynucleotide via a linker between the bicyclic peptide moiety and the polynucleotide. The linker can be any linker as described in the present disclosure. In some embodiments, the linker comprises ethylene glycol of the formula —(CH₂CH₂O)ₙ—, wherein n is 1-100, 1-60, 1-40, 1-30, 1-20, 1-10, 1-5, 10-20, 20-30, 20-25, 22-24. In some embodiments, n is 23 or 24. In some embodiments, the BiPPB is conjugated to the polynucleotide via a Diels-Alder coupling reaction between a free maleimide group linked to BiPPB and a diene group linked to the polynucleotide. In some embodiments, the diene group is linked to the 5′ end of the polynucleotide. In some embodiments, the diene group is linked to the 3′ end of the polynucleotide. In some embodiments, the diene group is linked to the polynucleotide via a terminal phosphate group. In some embodiments, the diene group is linked to the polynucleotide via a terminal phosphorothioate group. In some embodiments, the diene group is a Cm diene, wherein m is 4-10. In some embodiments, the diene group is a C₇ diene. In some embodiments, the diene group is a C₆ diene. In some embodiments, the agent is linked to the sense strand. In some embodiments, the agent is linked to the antisense strand. In some embodiments, the agent is linked to the sense strand comprising or consisting of SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, or SEQ ID NO: 54.

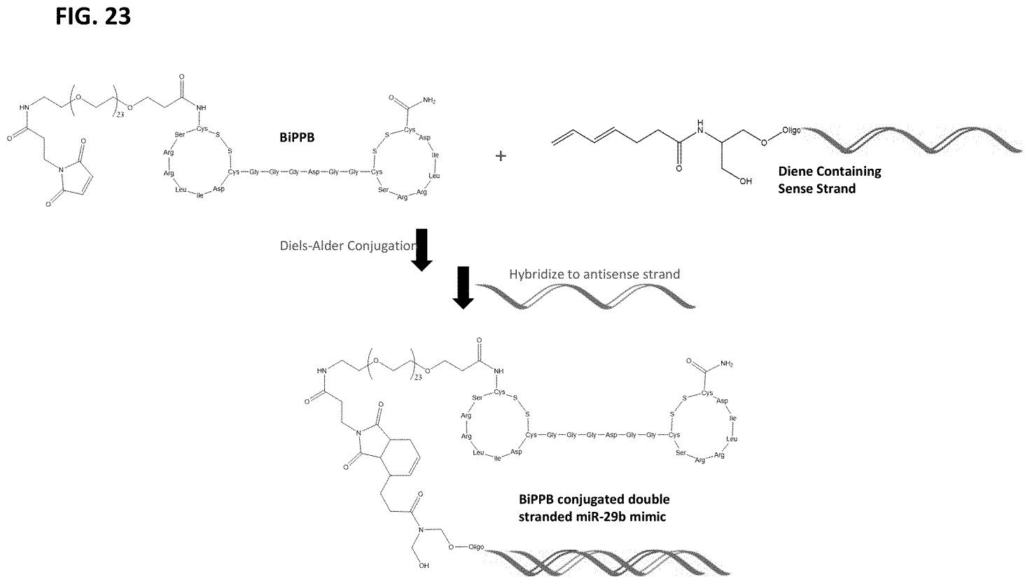

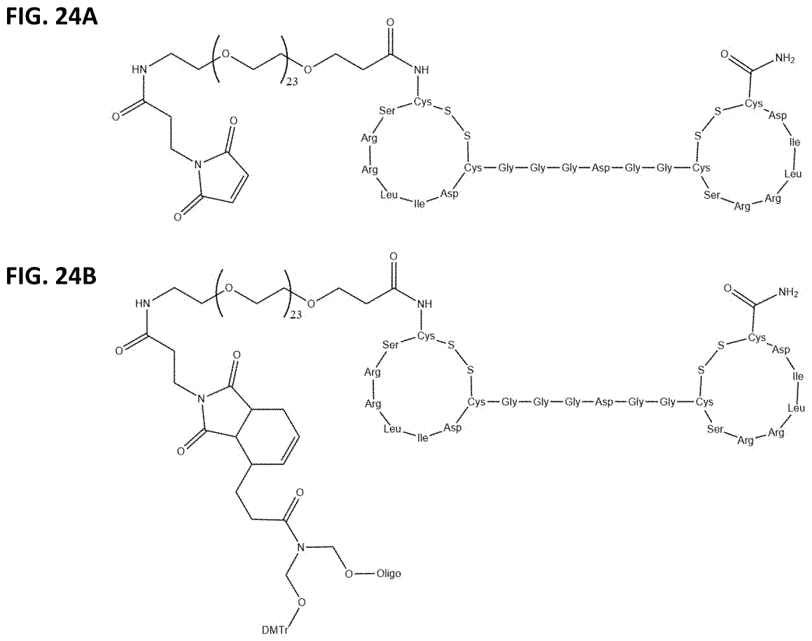

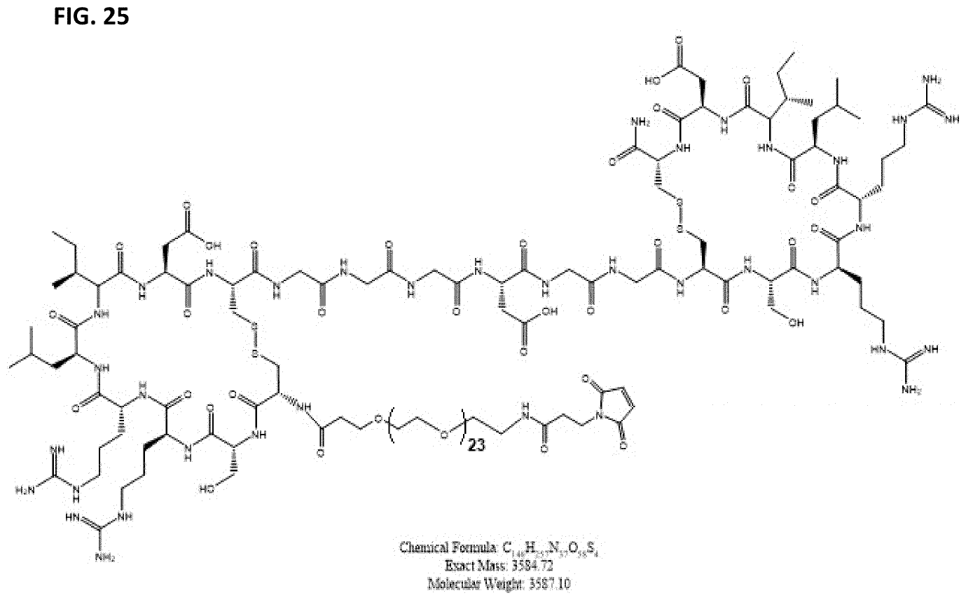

In some embodiments, the miR-29 mimic comprises an agent comprising a BiPPB with amino acid sequence CSRRLIDCGGGDGGCSRRLIDC, a sense strand polynucleotide comprising or consisting of SEQ ID NO: 42, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, SEQ ID NO: 53, or SEQ ID NO: 54, and a linker in between. In some embodiments, the BiPPB and part of the linker that undergo Diels-Alder coupling reaction are the same as those illustrated in FIG. 24A and/or FIG. 25. In some embodiments, the agent and linker and their conjugation to the polynucleotide are the same as those illustrated in FIG. 23 and/or FIG. 24B. In some embodiments, the linker has a chemical structure that is the same as or similar to the one illustrated in FIGS. 23 and 24A-B. In some embodiments, the linker is conjugated to the 5′ end of the polynucleotide via a phosphorothioate group. In some embodiments, the miR-29 mimic comprises an agent linked to a second strand as in SEQ ID NO: 66, SEQ ID NO: 67, SEQ ID NO: 68, SEQ ID NO: 69, SEQ ID NO: 71, SEQ ID NO: 97, or SEQ ID NO: 98. In some embodiments, the miR-29 mimic further comprises an antisense strand comprising or consisting of SEQ ID NO: 55, SEQ ID NO: 74, or SEQ ID NO: 75. In some embodiments, the miR-29 mimic comprises an agent linked to a second strand as in SEQ ID NO: 69, and a first strand comprising or consisting of a polynucleotide sequence of SEQ ID NO: 55. In some embodiments, the miR-29 mimic comprises an agent linked to a second strand as in SEQ ID NO: 71, and a first strand comprising or consisting of a polynucleotide sequence of SEQ ID NO: 55. In some embodiments, the miR-29 mimic comprises an agent linked to a second strand as in SEQ ID NO: 97, and a first strand comprising or consisting of a polynucleotide sequence of SEQ ID NO: 55. In some embodiments, the miR-29 mimic comprises an agent linked to a second strand as in SEQ ID NO: 98, and a first strand comprising or consisting of a polynucleotide sequence of SEQ ID NO: 55. In some embodiments, the miR-29 mimic comprises an agent linked to a second strand as in SEQ ID NO: 66, and a first strand comprising or consisting of a polynucleotide sequence of SEQ ID NO: 74. In some embodiments, the miR-29 mimic comprises an agent linked to a second strand as in SEQ ID NO: 67, and a first strand comprising or consisting of a polynucleotide sequence of SEQ ID NO: 74. In some embodiments, the miR-29 mimic comprises an agent linked to a second strand as in SEQ ID NO: 68, and a first strand comprising or consisting of a polynucleotide sequence of SEQ ID NO: 75.

In some embodiments, the peptide is a PDGF-R binding bicyclic peptide (BiPPB). In some embodiment, the BiPPB is a peptide as in FIG. 25.

In some embodiments, the peptide is a receptor-binding peptide. In some embodiments, the peptide binds a receptor involved in inflammation,

In some embodiments, the peptide is a PDGF receptor-binding peptide, for example wherein the PDGF receptor-binding peptide is derived from PDGF-beta.

In some embodiments, the agent is a small molecule. In some embodiments, the small molecule is a retinoid. In some embodiments, the retinoid is an all trans retinoid. In some embodiments, the retinoid is retinoic acid.

In some embodiments, the agent is a fatty acid. In some embodiments, the fatty acid is a long chain fatty acid. In some embodiments, the long fatty acid comprises 8-30 carbons. In some embodiments, the long fatty acid is a long-chain docosanoic acid (DCA)

In some embodiments, the agent is conjugated to the first strand. In some embodiments, the agent is conjugated to the 5′-end of the first strand. In some embodiments, the agent is conjugated to the 3′-end of the first strand. In some embodiments, the agent is conjugated to the second strand. In some embodiments, the agent is conjugated to the 5′-end of the second strand. In some embodiments, the agent is conjugated to the 3′-end of the second strand.

In some embodiments, the agent is conjugated to the miR-29 mimic via a linker. As used herein, a linker is a linear or branched molecule that covalently connects two or more chemical moieties such as a polynucleotide and a peptide, a polynucleotide and a small molecule and a polynucleotide and a fatty acid.

In some embodiments, linkers also may contain functional groups at two or more locations to enable the covalent attachment of chemical moieties.

Linkers include, but are not limited to, polyethyleneglycol (PEG), ethylene glycol and defined oligomers of ethylene glycol of the formula —(CH₂CH₂O)ₙ— where n is a defined integer, peptides, poly amino acids, oligomers of amino acids of defined lengths, polyamines such as polyethylenimine, spermine, spermidine, dedrimeric polyamines, carbon chain linker of the general formula —(CH₂)ₘ— where m is a defined integer, DNA, RNA thioethers and combinations of these.

Linkers also include glycine polymers (G)n, glycine-serine polymers (including, for example, (GS)n, (GSGGS)n and (GGGS)n, where n is an integer of 1-20, glycine-alanine polymers, alanine-serine polymers, and other flexible linkers known in the art.

In some embodiments, the agent is conjugated to the polynucleotide via cholesterol. In some embodiments the agent is conjugated at an internal position of the first strand. In some embodiments the agent is conjugated at an internal position of the second strand.

In some embodiments, ligand can be conjugated to nucleobases, sugar moieties, or internucleosidic linkages of nucleic acid molecules. Conjugation to purine nucleobases or derivatives thereof can occur at any position including, endocyclic and exocyclic atoms. In some embodiments, the 2-, 6-, 7-, or 8-positions of a purine nucleobase are attached to a conjugate moiety. Conjugation to pyrimidine nucleobases or derivatives thereof can also occur at any position. In some embodiments, the 2-, 5-, and 6-positions of a pyrimidine nucleobase can be substituted with a conjugate moiety. Conjugation to sugar moieties of nucleosides can occur at any carbon atom. Example carbon atoms of a sugar moiety that can be attached to a conjugate moiety include the 2′, 3′, and 5′ carbon atoms. The 1′ position can also be attached to a conjugate moiety, such as in an abasic residue. Intemucleosidic linkages can also bear conjugate moieties. For phosphorus-containing linkages (e.g., phosphodiester, phosphorothioate, phosphorodithioate, phosphoroamidate, and the like), the conjugate moiety can be attached directly to the phosphorus atom or to an O, N, or S atom bound to the phosphorus atom. For amine- or amide-containing intemucleosidic linkages (e.g., PNA), the conjugate moiety can be attached to the nitrogen atom of the amine or amide or to an adjacent carbon atom.

Table 7 provides exemplary polynucleotide conjugates of the disclosure, wherein the polynucleotide comprises a synthetic double stranded miR-29 mimic and the agent comprises an agent of the disclosure.

TABLE 7
Exemplary synthetic microRNA-29b mimics and non-targeting control sequences

| Mimic | Mimic | Sense/second/passenger strand | Antisense/first/guide strand |
|---|---|---|---|
| # | Name | SEQ ID NO | (SEQ ID NO) |
| 1 | 5′ | SEQ ID NO: 66: 5′-BPM.C6D. | SEQ ID NO: 74: 5′-p.fU.rA.rG.fC.rA. |
| | BiPPB- | mA.mA.mC.rA.mC.mU.rG.mU. | fC.fC.rA.fU.fU.fU.rG.rA.rA.rA.fU.fC.rA. |
| | Lightly | mU.mU.rA.mC.rA.rA.rA.mU.rG. | rG.fU.rG.fU.fUs.rUs.rU-3′ |
| | Modified | rG.mU.mC.mC.mU.rA-3′ | |
| 2 | 3′ | SEQ ID NO: 67: 5′-mA.mA.mC. | SEQ ID NO: 74: 5′-p.fU.rA.rG.fC.rA.fC. |
| | BiPPB- | rA.mC.mU.rG.mU.mU.mU.rA. | fC.rA.fU.fU.fU.rG.rA.rA.rA.fU.fC.rA.rG. |
| | Lightly | mC.rA.rA.rA.mU.rG.rG.mU.mC. | fU.rG.fU.fUs.rUs.rU-3′ |
| | Modified | mC.mU.rAs.C6D-sup.BPM-3′ | |
| 3 | 5′BiPPB- | SEQ ID NO: 68: 5′-BPM.C6D. | SEQ ID NO: 75: 5′-p.mUs.fAs.mG.fC. |
| | Fully | fAs.mAs.fC.mA.fC.mU.fG.mU. | mA.fC.mC.fA.mU.fU.mU.mG.mA.fA. |
| | modified- | fU.mU.fA.fC.fA.mA.fA.mU.fG. | mA.fU.mC.fA.mG.fU.mG.fU.mUs.mUs. |
| | 1 | mG.fU.mC.fC.mU.fA-3′ | mU-3′ |
| 4 | 5′BiPPB- | SEQ ID NO: 69: 5′-BPM.C6D. | SEQ ID NO: 55: 5′-mUs.fAs.mG.fC.mA. |
| | Fully | fAs.mAs.fC.mA.fC.mU.fG.mU. | fC.mC.fA.mU.fU.mU.mG.mA.fA.mA.fU. |
| | modified- | fU.mU.fA.fC.fA.mA.fA.mU.fG. | mC.fA.mG.fU.mG.fU.mUs.mUs.mU-3′ |
| | 2 | mG.fU.mC.fC.mU.fA-3′ | |
| 5 | 5′BiPPB- | SEQ ID NO: 70: 5′- | SEQ ID NO: 76: 5′- |
| | Non- | BPM.C6D.fAs.mUs.fG.mU.fA.mC. | p.mUs.fUs.mG.fA.mG.fC.mA.fA.mU.fU. |
| | Targeting | fC.mU.fG.mA.fA.mU.fU.mG.fC. | mC.fA.mC.fG.mU.fU.mC.fA.mUs.mUs. |
| | Control | mA.fC.mA.fA | mU |
| 6 | 3′BiPPB- | SEQ ID NO: 71: 5′-fAs.mAs.fC. | SEQ ID NO: 55: 5′-mUs.fAs.mG.fC.mA. |
| | Fully | mA.fC.mU.fG.mU.fU.mU.fA.fC. | fC.mC.fA.mU.fU.mU.mG.mA.fA.mA.fU. |
| | modified | fA.mA.fA.mU.fG.mG.fU.mC.fC. | mC.fA.mG.fU.mG.fU.mUs.mUs.mU-3′ |
| | | mU.fAs.C7D-sup.BPM-3′ | |
| 12 | 5′BiPPB- | SEQ ID NO: 97: 5′-hBPM.C7Ds. | SEQ ID NO: 55: 5′-mUs.fAs.mG.fC.mA. |
| | Fully | fAs.mAs.fC.mA.fC.mU.fG.mU. | fC.mC.fA.mU.fU.mU.mG.mA.fA.mA.fU. |
| | modified- | fU.mU.fA.fC.fA.mA.fA.mU.fG. | mC.fA.mG.fU.mG.fU.mUs.mUs.mU-3′ |
| | 3 | mG.fU.mC.fC.mU.fA-3′ | |
| 13 | 5′BiPPB- | SEQ ID NO: 98: 5′-mBPM.C7Ds. | SEQ ID NO: 55: 5′-mUs.fAs.mG.fC.mA. |
| | Fully | fAs.mAs.fC.mA.fC.mU.fG.mU. | fC.mC.fA.mU.fU.mU.mG.mA.fA.mA.fU. |
| | modified- | fU.mU.fA.fC.fA.mA.fA.mU.fG. | mC.fA.mG.fU.mG.fU.mUs.mUs.mU-3′ |
| | 4 | mG.fU.mC.fC.mU.fA-3′ | |
| 8 | 3′chol- | SEQ ID NO: 54: 5′-mA.mA.mC. | SEQ ID NO: 77: 5′-fU.rA.rG.fC.rA.fC. |
| | lightly | rA.mC.mU.rG.mU.mU.mU.rA. | fC.rA.fU.fU.fU.rG.rA.rA.rA.fU.fC.rA.rG. |
| | modified | mC.rA.rA.rA.mU.rG.rG.mU.mC. | fU.rG.fU.fUs.rUs.rU-3′ |
| | | mC.mU.rA.chol6-3′ | |
| 7 | RA-Fully | SEQ ID NO: 72: 5′-fAs.mAs.fC. | SEQ ID NO: 55: 5′-mUs.fAs.mG.fC.mA. |
| | modified | mA.fC.mU.fG.mU.fU.mU.fA.fC. | fC.mC.fA.mU.fU.mU.mG.mA.fA.mA.fU. |
| | | fA.mA.fA.mU.fG.mG.fU.mC.fC. | mC.fA.mG.fU.mG.fU.mUs.mUs.mU-3′ |
| | | mU.fAs.PTA.RET-3′ | |
| 9 | 3′chol- | SEQ ID NO: 53: 5′-fAs.mAs.fC. | SEQ ID NO: 55: 5′-mUs.fAs.mG.fC.mA. |
| | fully | mA.fC.mU.fG.mU.fU.mU.fA.fC. | fC.mC.fA.mU.fU.mU.mG.mA.fA.mA.fU. |
| | modified | fA.mA.fA.mU.fG.mG.fU.mC.fC. | mC.fA.mG.fU.mG.fU.mUs.mUs.mU-3′ |
| | | mU.fAs.chol6-3′ | |
| 10 | Un- | SEQ ID NO: 45: 5′-fAs.mAs.fC. | SEQ ID NO: 55: 5′-mUs.fAs.mG.fC.mA. |
| | congujated | mA.fC.mU.fG.mU.fU.mU.fA.fC. | fC.mC.fA.mU.fU.mU.mG.mA.fA.mA.fU. |
| | fully | fA.mA.fA.mU.fG.mG.fU.mC.fC. | mC.fA.mG.fU.mG.fU.mUs.mUs.mU-3′ |
| | modified | mU.fA-3′ | |
| 11 | DCA- | SEQ ID NO: 73: 5′-fAs.mAs.fC. | SEQ ID NO: 55: 5′-mUs.fAs.mG.fC.mA. |
| | Fully | mA.fC.mU.fG.mU.fU.mU.fA.fC. | fC.mC.fA.mU.fU.mU.mG.mA.fA.mA.fU. |
| | Modified | fA.mA.fA.mU.fG.mG.fU.mC.fC. | mC.fA.mG.fU.mG.fU.mUs.mUs.mU-3′ |
| | | mU.fAs.DCA-3′ | |

### III. Methods of Making Polynucleotide Conjugates

Polynucleotide conjugates can be produced by solution phase reaction of a conjugate with a polynucleotide where each of them contain a functional group that react with each other under appropriate conditions to produce a covalent linkage. Examples of complementary pairs of reaction group include: an activated carboxylic acid (e.g an active ester) and an amine group; an Amine and an isothiocyanate; an aldehyde and an amine; an aldehyde and a hydrazine; an azide and a phosphine; azide and an alkyne; a diene and a dienophile; a thiol and a maleimide; a thiol and a thiol

Polynucleotide conjugates can also produce using polynucleotide solid phase synthesis protocols. For incorporation into the 3′ end of the polynucleotide an appropriately protected succinate of the conjugate can be used to derivatize a solid support such controlled pore glass (CPG) containing long chain alkylamine group. This derivatized CPG can then be used for synthesis of the polynucleotide conjugate. Alternately, the conjugate can be synthesized as an appropriately protected amidite that can be coupled during the solid phase synthesis of polynucleotides to incorporate the conjugate at the 3′ end, the 5′ end or an internal position. Such amidites includes conjugate covalently attached to an abasic backbone such as a serinol group as well as a nucleoside wherein the conjugate is covalently attached to the ribose, the nucleobase or to the backbone phosphate groups.

### IV. Methods of Use of Polynucleotide Conjugates

Provided herein are methods of use of the polynucleotide conjugates provided herein.

As used herein, the term “subject” refers to any vertebrate including, without limitation, humans and other primates (e.g., chimpanzees, cynomologous monkeys, and other apes and monkey species), farm animals (e.g., cattle, sheep, pigs, goats and horses), domestic mammals (e.g., dogs and cats), laboratory animals (e.g., rabbits, rodents such as mice, rats, and guinea pigs), and birds (e.g., domestic, wild and game birds such as chickens, turkeys and other gallinaceous birds, ducks, geese, and the like). In some embodiments, the subject is a mammal. In exemplary embodiments, the subject is a human.

A. Therapeutic Indications

The present disclosure provides methods of treating, ameliorating, or preventing one or more conditions in a subject in need thereof comprising administering to the subject a therapeutically effective amount of at least one of the polynucleotide conjugates described herein.

In some embodiments, the present disclosure provides methods of treating, ameliorating, or preventing fibrotic conditions in a subject in need thereof comprising administering to the subject a therapeutically effective amount of at least one of the polynucleotide conjugates described herein. Fibrotic conditions that may be treated using polynucleotide conjugates of the disclosure include, but are not limited to, skeletal muscle fibrosis, diabetic fibrosis, pulmonary (lung) fibrosis, cardiac fibrosis, cutaneous fibrosis (skin, dermal), liver fibrosis, renal fibrosis, and ocular fibrosis.

In some embodiments, pulmonary fibrosis includes or may be caused by idiopathic pulmonary fibrosis, scleroderma ILD, rheumatoid arthritis ILD, bronchiolitis obliterans syndrome, chronic obstructive pulmonary disease, bronchopulmonary dysplasia, or any combination thereof.

In some embodiments, cutaneous fibrosis includes or may be caused by cutaneous sclerosis, systemic sclerosis (scleroderma), dystrophic epidermolysis bullosa, keloid scar, keloids, hypertrophic scar, hand/joint/tendon fibrosis, and Peyronie's disease, or any combination thereof.

In some embodiments, cardiac fibrosis includes or may be caused by myocardial infarction, congestive heart failure, myocardial fibrosis, or any combination thereof.

In some embodiments, Liver fibrosis includes or may be caused by NASH, Cirrhosis, 1Viral (HBV/HCV), or any combination thereof.

In some embodiments, renal fibrosis may include but is not limited to diabetic nephropathy, IgA nephropathy, lupus nephitis, Non-lupus chronic kidney disease, or any combination thereof.

In some embodiments, ocular fibrosis includes or may be caused by fibrosis of the cornea, retina, trabecular meshwork and/or pterygium, Fuch's endothelial corneal dystrophy, glaucoma/trabeculectomy bleb, age related macular degeneration, dieabetic retinopathy, or any combination thereof.

In some embodiments, the present disclosure provides methods of treating, ameliorating, or preventing any one or more of the following diseases/indications: muscular dystrophy, Dupuytren's contractures, tendinopathies, osteoarthritis, inflammatory bowel disease, or any combination thereof, comprising administering to the subject a therapeutically effective amount of at least one of the polynucleotide conjugates described herein.

In some embodiments, the present disclosure provides methods of treating, ameliorating, or preventing inflammation in a subject in need thereof comprising administering to the subject a therapeutically effective amount of at least one of the polynucleotide conjugates described herein. In some embodiments, the present disclosure provides methods of treating, ameliorating, or preventing inflammatory bowel disease.

B. Regulation of Gene Expression

The polynucleotide conjugates of the disclosure are also useful for regulating the expression of genes, e.g. extracellular matrix genes in a cell. In some embodiments, the present disclosure provides methods of regulating at least one gene in a cell comprising contacting the cell with one or more polynucleotide conjugates disclosed herein.

In some embodiments, the present disclosure provides methods of downregulating the expression of the gene associated with fibrosis in a cell comprising contacting the cell with one or more polynucleotide conjugates disclosed herein. In some embodiments, the regulated gene is Col1a1, Col1a2, Col3a1, Eln, Tgfb2, Tgfb3, Mfap 2, Igf1, Ctgf, Il13, Ccr2, Itga1, Cdh1, Col4a5, Smad3, Itgb6, Wnt11, Mfap2, Sparc, Acta2, Plau, Ccr2, Col4a1, Col2a1, Plat, Col4a2, Egf, Eln, Col5a2, Ctgf, Thbs2, Ccl2, Plau, Fstl1, Col5a1, Fbn1, or any combination thereof.

In some embodiments, the present disclosure provides methods of downregulating the expression of at least one gene associated with collagen synthesis in a cell comprising contacting the cell with one or more polynucleotide conjugates disclosed herein.

In some embodiments, the present disclosure provides methods of downregulating the expression of the growth factor gene in a cell comprising contacting the cell with one or more polynucleotide conjugates disclosed herein. In some embodiments, the regulated gene is TGF-b2, TGF-b3, EGF, IGF1, IGF2, IGFBP5, PDGFA, PDGFC, or any combination thereof.

In some embodiments, the present disclosure provides methods of downregulating the expression of the collagen gene (regulating collagen transcription/translation) in a cell comprising contacting the cell with one or more polynucleotide conjugates disclosed herein. In some embodiments, the regulated gene is COL1A1, 1A2, 2A1, 3A1, 4A1, 4A2, 4A5, 5A1, 5A2, 5A3, 6A4, 6A5, 6A6, 8A1, 8A2, 9A1, 11A1, 12A1, 14A1, 22A1, 28A1, or any combination thereof.

In some embodiments, the present disclosure provides methods of downregulating the expression of the gene associated with post-translational modification and/or triple helix formation in a cell comprising contacting the cell with one or more polynucleotide conjugates disclosed herein. In some embodiments, the regulated gene associated with post-translational modification and triple helix formation is HSP47, P4HA2, P4HA3, PLOD2, or any combination thereof.

In some embodiments, the present disclosure provides methods of downregulating the expression of the gene associated with N- and C-terminal cleavage and secretion in a cell comprising contacting the cell with one or more polynucleotide conjugates disclosed herein. In some embodiments, the regulated gene associated with N- and C-terminal cleavage and secretion is PCOLCE, PCOLCE2, or any combination thereof.

In some embodiments, the present disclosure provides methods of downregulating the expression of the associated with fibril cross-linking in a cell comprising contacting the cell with one or more polynucleotide conjugates disclosed herein. In some embodiments, the regulated gene with fibril cross-linking is LOX, LOXL2, or any combination thereof.

In some embodiments, the present disclosure provides methods of downregulating mature collagen fibrils in a cell, comprising contacting the cell with one or more polynucleotide conjugates disclosed herein.

In some embodiments, administration of any one of the polynucleotide conjugates of the present disclosure to a subject reduces the expression or activity one or more extracellular matrix genes in cells of the subject. In other embodiments, administration of any one of the polynucleotide conjugates of the present disclosure to a subject reduces the expression or activity one or more collagen synthesis genes in cells of the subject. In some embodiments, administration of any one of the polynucleotide conjugates of the present disclosure to a subject reduces the expression or activity of at least one gene associated with collagen synthesis in cells of the subject. In some embodiments, administration of any one of the polynucleotide conjugates of the present disclosure to a subject reduces collagen transcription/translation in cells of the subject.

In some embodiments, administration of any one of the polynucleotide conjugates of the present disclosure to a subject reduces the expression of at least one growth factor gene in cells of the subject. In particular embodiments, the at least one growth factor gene is TGF-b2, TGF-b3, EGF, IGF1, IGF2, IGFBP5, PDGFA or PDGFC.

In some embodiments, administration of any one of the polynucleotide conjugates of the present disclosure to a subject reduces the expression of the collagen gene (e.g. collagen transcription/translation) in cells of the subject. In some embodiments, the regulated collagen gene is COL1A1, 1A2, 2A1, 3A1, 4A1, 4A2, 4A5, 5A1, 5A2, 5A3, 6A4, 6A5, 6A6, 8A1, 8A2, 9A1, 11A1, 12A1, 14A1, 22A1, 28A1, or any combination thereof.

In some embodiments, administration of any one of the polynucleotide conjugates of the present disclosure to a subject reduces the expression of the gene associated with post-translational modification and/or triple helix formation in cells of the subject. In particular embodiments, the post-translational modification and triple helix formation is HSP47, P4HA2, P4HA3, PLOD2, or any combination thereof.

In some embodiments, administration of any one of the polynucleotide conjugates of the present disclosure to a subject reduces the expression of the gene associated with N- and C-terminal cleavage and secretion in cells of the subject. In particular embodiments, the gene associated with N- and C-terminal cleavage and secretion is PCOLCE, PCOLCE2, or any combination thereof.

In some embodiments, administration of any one of the polynucleotide conjugates of the present disclosure to a subject reduces the expression of the gene associated with fibril cross-linking in cells of the subject. In particular embodiments, the gene associated with fibril cross-linking is LOX, LOXL2, or any combination thereof.

In some embodiments, administration of any one of the polynucleotide conjugates of the present disclosure to a subject downregulates mature collagen fibrils in cells and/or genes associated with fibril cross-linking of the subject.

In yet other embodiments, administration of polynucleotide conjugates to a subject up-regulates the expression or activity one or more genes involved in the skin development, epidermis development, ectoderm development and cellular homeostasis. Cells of the subject where the expression or activity of various genes is regulated by polynucleotide conjugates of the disclosure include fibroblasts and epidermal cells. In some embodiments, administration of polynucleotide conjugates down-regulates inflammatory responses associated with fibrosis. For example, administration of polynucleotide conjugates reduces the levels of pro-inflammatory cytokines such as IL-12, IL-4, GCSF, and TNF-α in fibrosis patients. Administration of polynucleotide conjugates may also reduce infiltration of immune effector cells such as neutrophils, lymphocytes, monocytes, and macrophages in fibrotic tissues or organs.

In certain embodiments, the present disclosure provides methods of regulating the expression of one or more extracellular matrix genes in a cell comprising contacting the cell with a polynucleotide conjugate of the present disclosure. In certain embodiments, the present disclosure provides methods of regulating the expression of one or more extracellular matrix genes in a subject comprising administering to the subject a polynucleotide conjugate of the present disclosure. In some embodiments, the extracellular matrix genes is elastin (ELN), fibrillin 1 (FBN1), collagen type I α1 (COL1A1), collagen type I α2 (COL1A2), collagen type III α1 (COL3A1), collagen type IV α4 (COL4A4), collagen type V α3 (COL5A3), collagen type XI α1 (COL11A1), collagen type V α1 (COL5A1), or collagen type IV α5 (COL4A5).

### V. Pharmaceutical Compositions

The present disclosure also provides pharmaceutical compositions comprising a therapeutically effective amount of one or more polynucleotide conjugates according to the disclosure or a pharmaceutically acceptable salt thereof, and a pharmaceutically acceptable carrier or excipient. In some embodiments, the pharmaceutical compositions of the disclosure comprise a therapeutically effective amount of at least two polynucleotide conjugates of the disclosure and a pharmaceutically acceptable carrier or excipient.

### VI. Routes of Administration

The disclosure also encompasses embodiments where additional therapeutic agents may be administered along with polynucleotide conjugates. The additional therapeutic agents may be administered concurrently but in separate formulations or sequentially. In other embodiments, additional therapeutic agents may be administered at different times prior to after administration of polynucleotide conjugates.

The polynucleotide conjugates of the disclosure, or the pharmaceutical compositions thereof may be administered via one or more of the following routes of administration: intravenous, intraocular, intravitreal intramuscular, subcutaneous, topical, oral, transdermal, intraperitoneal, intraorbital, by implantation, by inhalation, intrathecal, intraventricular, via the ear, or intranasal.

### VII. Kits and Articles of Manufacture

The present disclosure provides kits comprising any one or more of the polynucleotide conjugates described herein, or pharmaceutical compositions thereof.

The present disclosure also provides articles of manufacture comprising any one of the polynucleotide conjugates, pharmaceutical compositions or kits described herein.

The following examples are included for illustrative purposes and are not intend to limit the scope of the disclosure.

All patent and non-patent documents referenced throughout this disclosure are incorporated by reference herein in their entirety for all purposes

### EXAMPLES

The miR-29 mimics referred to in the examples are provided in Table 7 above.

### Example 1—Synthesis of miR-29b Mimics Conjugated to PDGFR-Binding Bicyclic Peptide

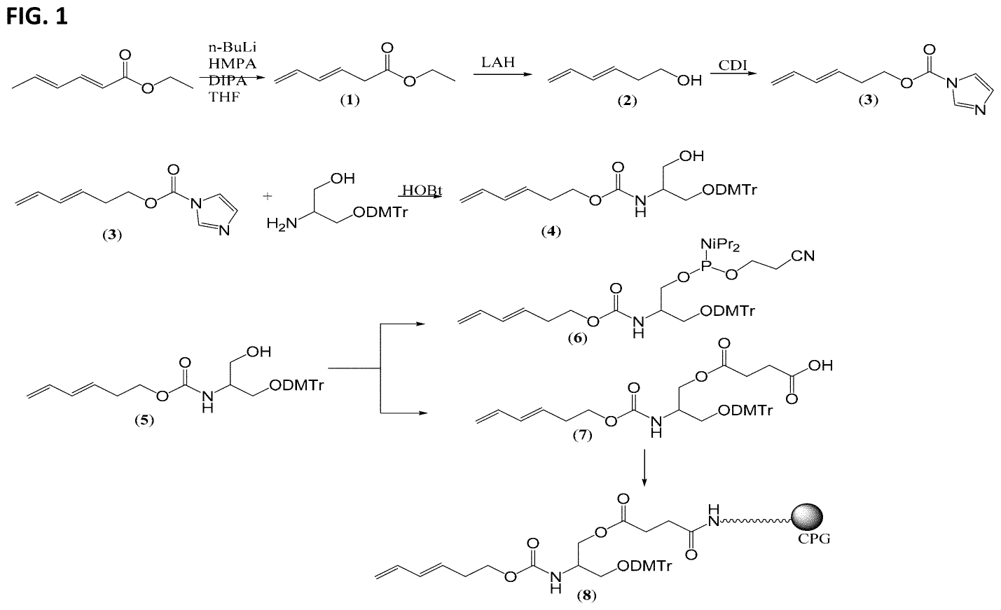

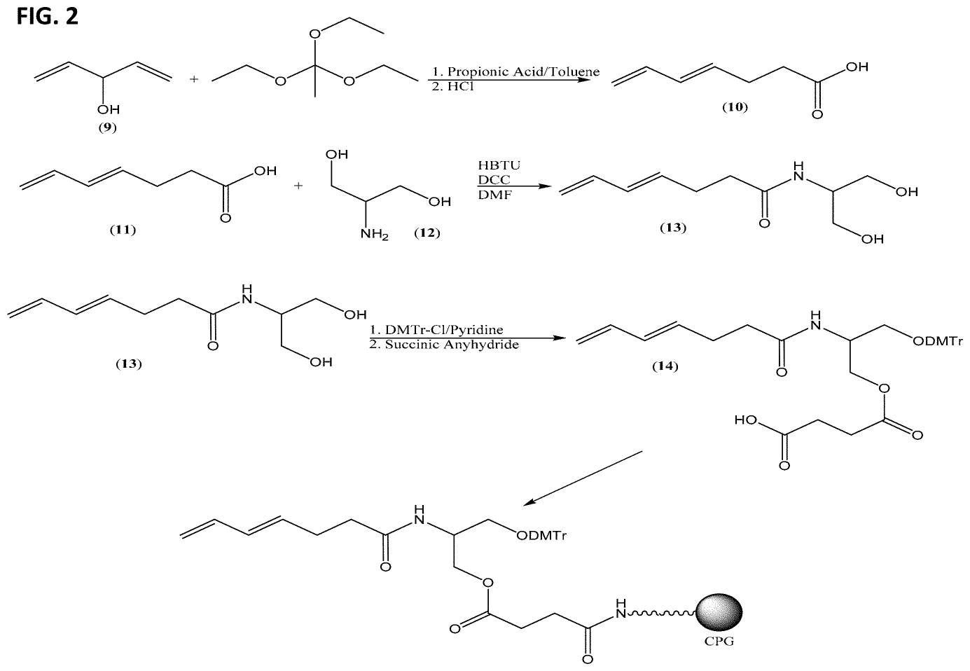

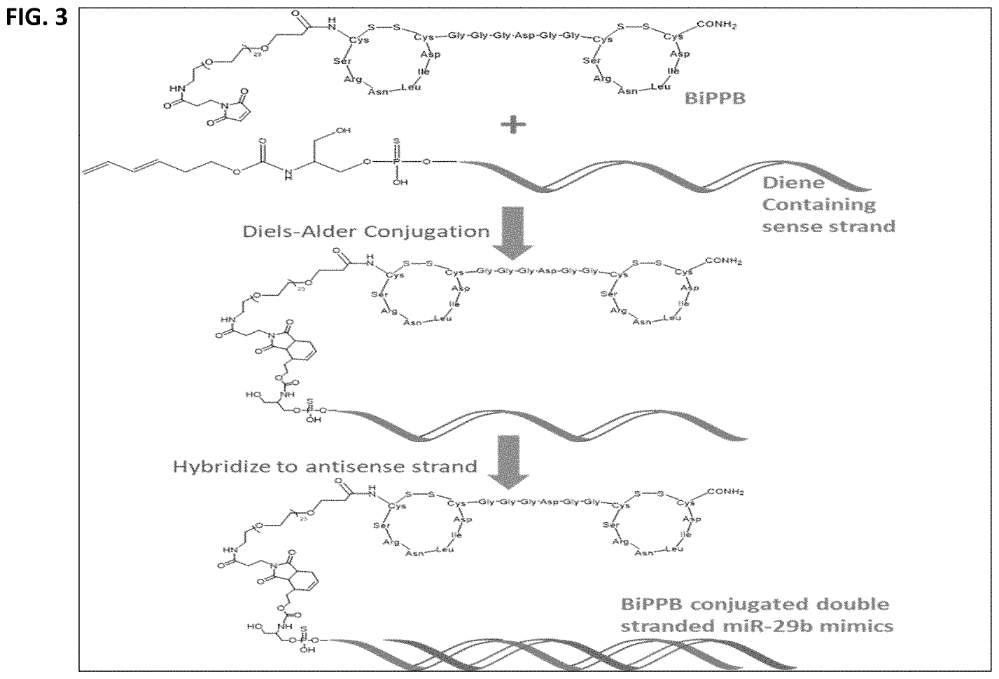

It was tested whether a PDGF-R binding bicyclic peptide (BiPPB) conjugated to miR-29b mimics could be used to deliver polynucleotide cargoes to target fibrotic tissues in idiopathic pulmonary fibrosis (IPF) and liver fibrosis mouse models. To prepare these synthetic conjugates, linkers containing a diene moiety were synthesized as shown in FIGS. 1 and 2. For incorporation at the 3′ end of polynucleotides, succinate derivatives (7 and 14) were loaded onto controlled pore glass (CPG) derivatized with long chain amine groups. For incorporation on the 5′ end of the sense strand of miR-29b mimic, molecule (5) was converted to an amidite (6) which was then incorporated as the last residue during solid phase synthesis of the polynucleotides. After HPLC purification, the diene containing polynucleotides were conjugated to BiPPB functionalized with a heterobifunctional linker containing a free maleimide group (purchased from Anaspec) via a Diels-Alder coupling reaction (FIG. 3). The conjugated polynucleotide was purified a second time by HPLC and hybridized to the antisense strand to produce the final constructs.

### Example 2—Synthesis of miR-29b Mimic Conjugated to Retinoic Acid

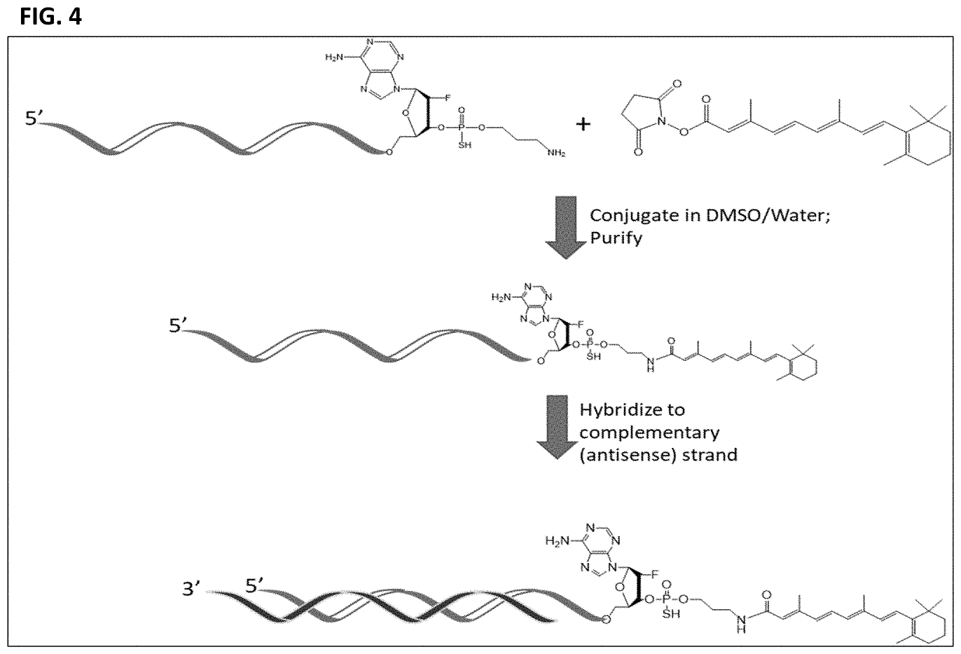

To produce a retinoic acid conjugated sense strand of a miR-29b mimic of the disclosure (M13373), the polynucleotide was first synthesized with a 3′ amine functional group (FIG. 4) using standard methods of polynucleotide synthesis and commercially available amidites and solid support. After HPLC purification, this polynucleotide was reacted with an excess of the NHS-ester derivative of retinoic acid (synthesized in-house), in a phosphate buffered solution. After completion of the reaction, as monitored by HPLC, the excess retinoic-NHS ester was removed using a 3000 Da MWCO filter. Retinoids demonstrate chemical instability towards oxidizing and acid conditions. The conjugation was carried out post-solid phase polynucleotide synthesis to avoid the oxidation or isomerization of the retinoic acid derivative. All steps were carried out in the dark to avoid photochemical degradation of retinoic acid. All materials were stored dry under an inert atmosphere prior to use. The polynucleotide was further purified by HPLC and hybridized with the antisense strand to produce the conjugated miR-29 mimic.

### Example 3—In Vivo Evaluation of BiPPB-Conjugated miR-29b Mimics in Idiopathic Pulmonary Fibrosis (IPF) Mouse Model

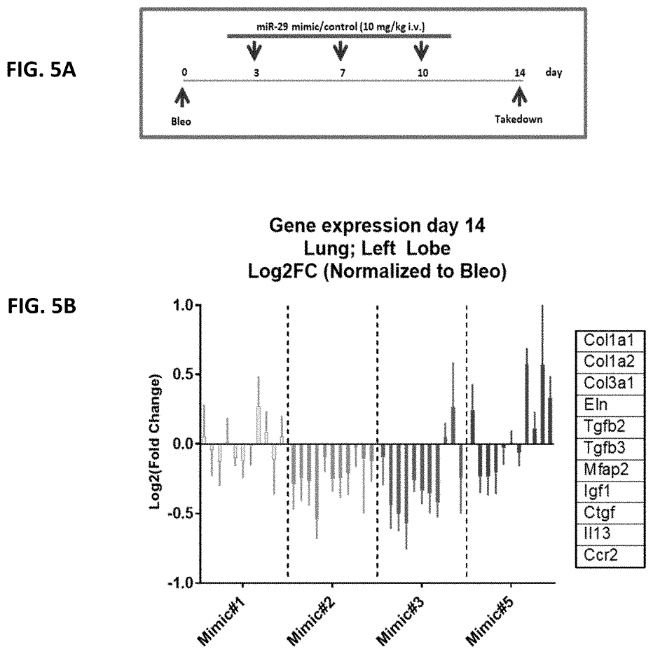

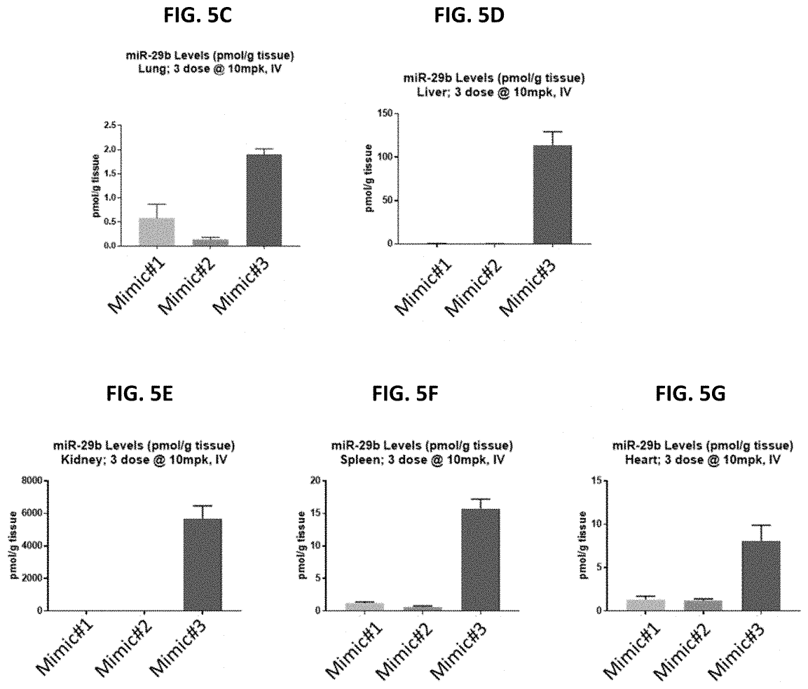

An initial study was carried out in a bleomycin-treated mouse model of IPF to evaluate the delivery of BiPPB conjugated synthetic miR-29b mimics to fibrotic tissues in the lung, and measure the subsequent down-regulation of miR-29b target genes. In this study, Male C5BL6 mouse were anesthetized with dexmedetomidine, at 1 mg/kg IP and subsequently given either one intra-tracheal dose of Bleomycin at 1.25 mg/kg in 50 mL saline or an equivalent volume of saline. Three miR-29b mimics with differing patterns of chemical modifications and points of BiPPB conjugation, along with a non-targeting control compound also conjugated to BiPPB were evaluated (Table 7, Mimic #1, Mimic #2, Mimic #3, Mimic #5). On days 3, 7 and 10 post-bleomycin treatment, animals were treated with miR-29b mimics at 10 mg/kg delivered intravenously. The animals were euthanized on day 14 for analyses (FIG. 5A).

FIG. 5B shows the expression levels of several miR-29b target genes in lung tissues that are associated with fibrosis. The levels are shown normalized to the control group which was treated with only saline, post-bleomycin. Interestingly, for the lightly modified mimics, while the 5′ BiPPB conjugate (Mimic #1) showed nearly no activity, the 3′ version (Mimic #2) demonstrated consistent downregulation of all the genes measured in this study. The fully modified mimic with a 5′ BiPPB conjugate (Mimic #3) also showed significant downregulation activity. The non-targeting control (Mimic #5), did not yield a consistent signature of gene regulation, thus indicating that the BiPPB peptide alone is not sufficient for down regulation of these genes.

The biodistribution of the miR-29b mimics was also measured in several tissue types using a hybridization based assay, as shown in FIGS. 5C-5G. Among these, the fully modified compound (Mimic #3) was detected in the lung (FIG. 5C), liver (FIG. 5D), kidney (FIG. 5E), heart (FIG. 5G) and spleen (FIG. 5F). The lightly modified compounds (Mimic #1 and Mimic #2) were detected in measurable amounts in the lung, kidney, heart and spleen, with their amounts being lower than Mimic #3.

### Example 4—Different Dosage Schedules of BiPPB-Conjugated miR-29b Mimics in IPF Model

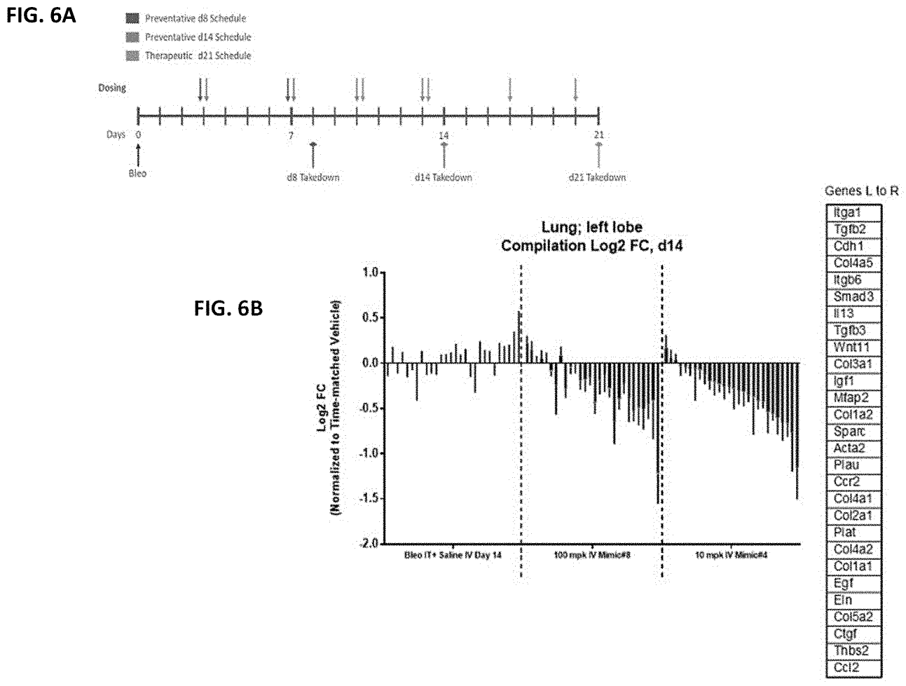

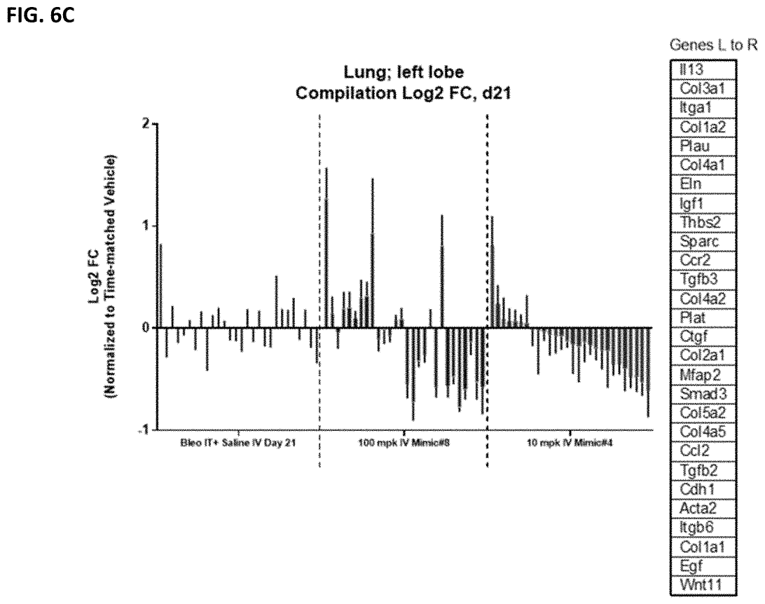

In a follow-up study, the BiPPB conjugated miR-29b mimics was re-evaluated in a Bleomycin mouse model with three different dosage schedules, as shown in FIG. 6A. These included two ‘preventive’ dosage paradigms, starting treatment with miR-29b mimic at day 3 post bleomycin administration and ending at days 7 and 13 with takedown of animals at days 8 and 14 respectively. These also corresponded to two and three doses of miR-29b mimic treatments respectively. A third dosage schedule, a ‘therapeutic’ dosage paradigm involved administration of four doses starting at day 10 post bleomycin treatment. The last dose was administered on day 20 followed by takedown at day 21 for analysis. In this experiment, a modified version of Mimic #3 lacking a phosphate group on the 5′ of the antisense strand was used (Mimic #4; Table 7). For this compound, doses of 10 mg/kg was used while Mimic #8, a cholesterol conjugated, lightly modified miR-29b mimic was used as a comparator at a 100 mg/kg dose level.

Downregulation of genes regulated by miR-29b was seen both with Mimic #4 and Mimic #8 with the day 14 dosage schedule (FIG. 6B). The day 21 dosage schedule also showed downregulation for a majority of the targets analyzed, particularly for Mimic #4 (FIG. 6C). However some genes were unchanged or slightly upregulated with respect to the untreated control group. For Mimic #8, the changes were more variable across the genes measured. In contrast, the day 8 dosage schedule showed no significant changes in the level of miR-29b targets (data not shown).

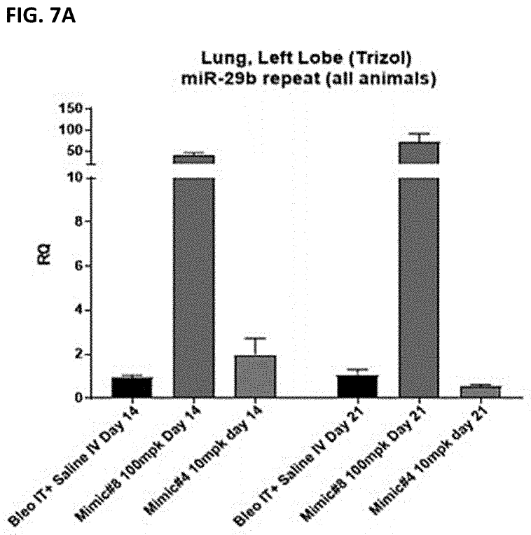

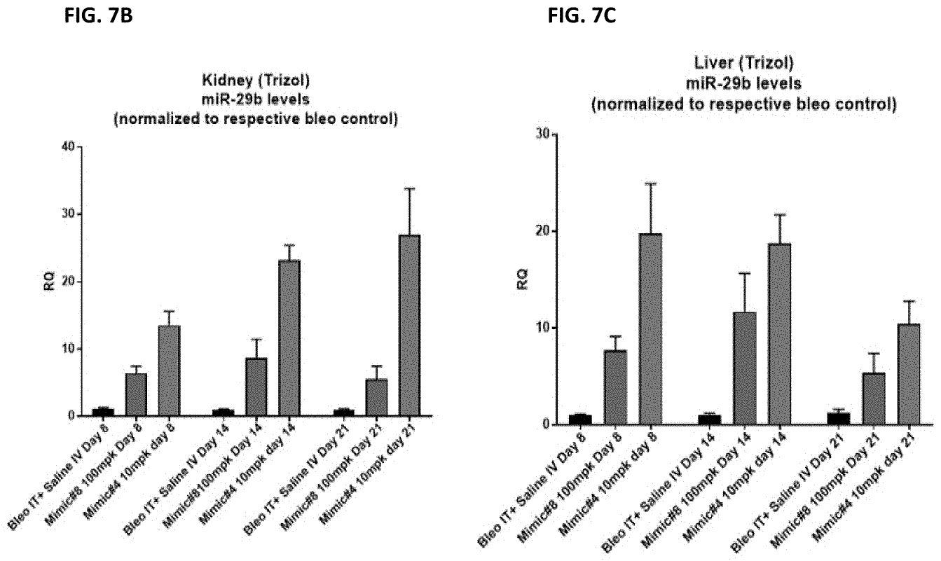

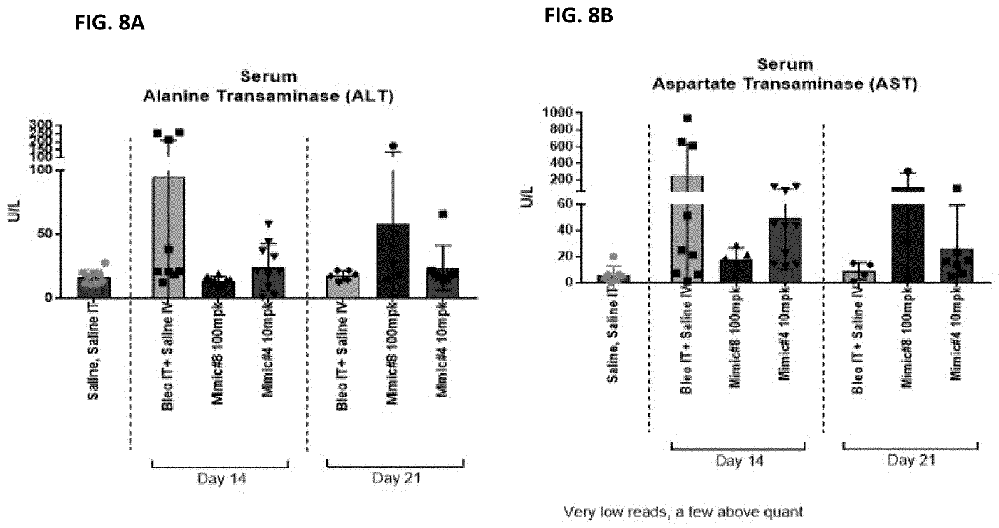

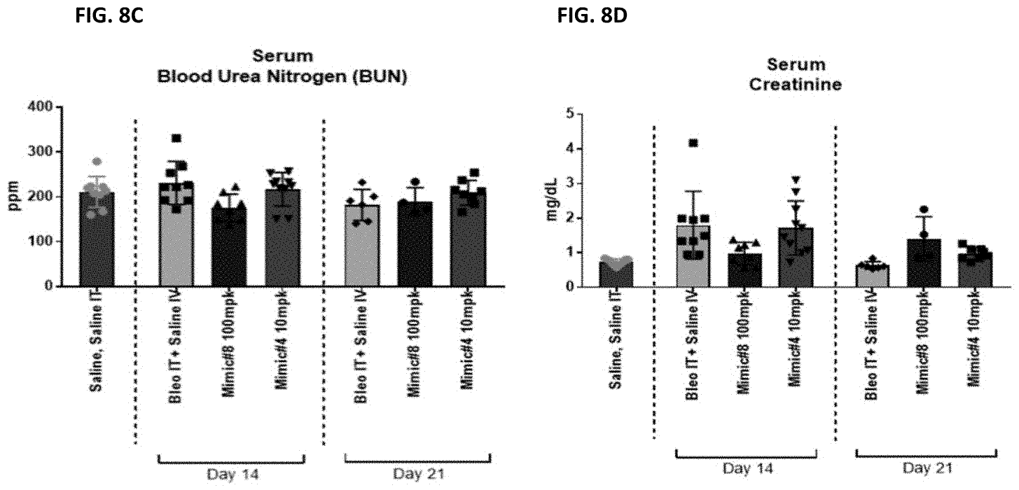

Measurement of miR-29b levels using a PCR based method of detection once again confirmed the presence of both Mimic #8 and Mimic #4 in the lung as well as much higher levels in the kidney and the liver (FIG. 7). However, despite high levels of accumulation of the compound in these organs no associated liver or kidney toxicity was found (FIG. 8). The liver enzymes, alanine transaminase and aspartate transaminase as well as the blood urea nitrogen and creatinine levels were found to be either similar or lower for the animal cohorts treated with Mimic #8 and Mimic #4 when compared to the Bleoymycin only group. Furthermore, comparison of these levels with those measured for a non-bleomycin treated, healthy animals group from a different study did not reveal any elevation in the levels of these toxicity parameters.

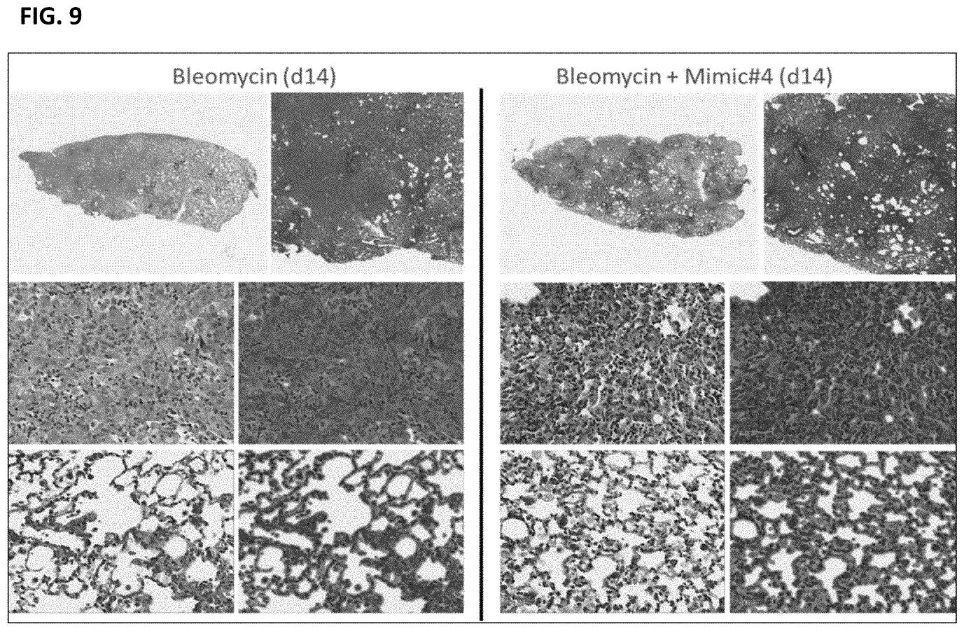

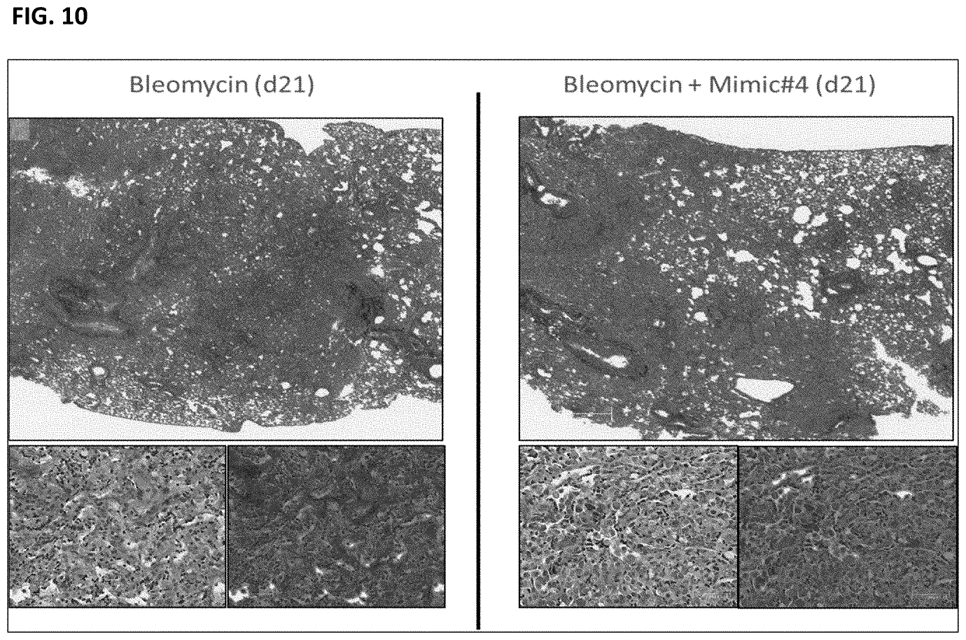

Histological analysis was performed on lung sections taken from animals in this study. Whole lungs were cut and trichrome stained to assess collagen deposition. Slides were then scanned, and collagen content was quantified as a percentage of total lung tissue. FIGS. 9 and 10 show representative images of the stained sections from day 14 and day 21 dosage schedules respectively for bleomycin only and bleomycin+Mimic #4 treated animals. The difference in airways between the two groups both at day 14 as well as day 21 is striking. Dense cellular areas associated with robust fibrosis can be seen in both groups from the blue staining. In both the preventative dosing paradigm (FIG. 9, day 14) and the therapeutic dosing paradigm (FIG. 10, day 21), the amount of area stained in blue is reduced, indicating that collagen deposition is lower, and therefore, that fibrosis is blunted.

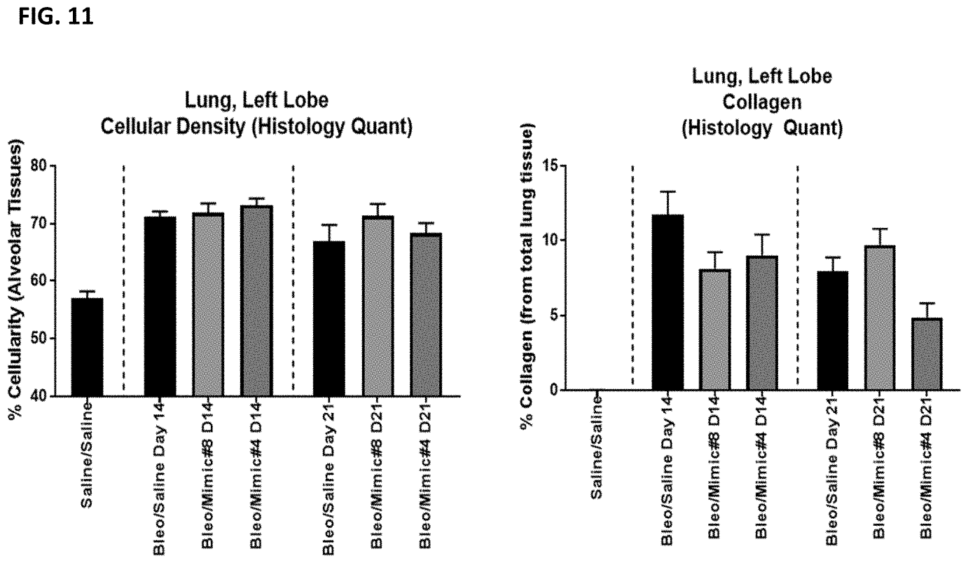

An automated algorithm was further used to obtain quantitative estimation of collagen and cellularity from these lung tissue sections. The results show that no difference in cellularity was observed upon treatment with either Mimic #8 or Mimic #4 (FIG. 11, left panel). However while bleomycin induced significant deposition of collagen in the lungs, total collagen was reduced for all the treated groups of animals with both compounds with the day 14 schedule (FIG. 11, right panel). Interestingly for the day 21 therapeutic dosage schedule, significant reduction in collagen was observed only for the Mimic #4 treated group.

### Example 5—In Vivo Evaluation of BiPPB-Conjugated and Retinoic Acid-Conjugated miR-29b Mimics in Liver Fibrosis Mouse Model

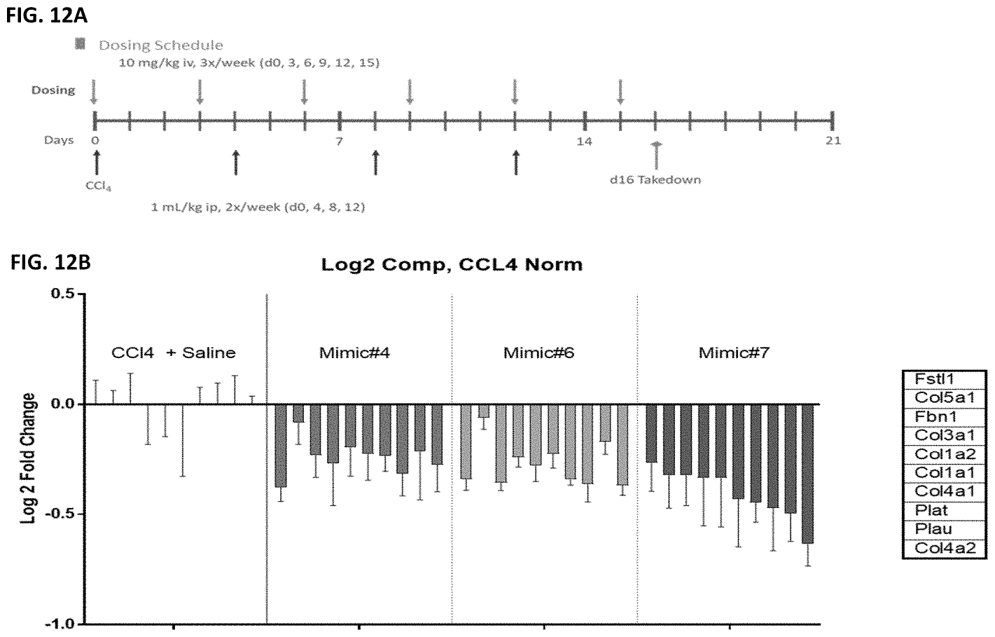

In this study the performance of BiPPB and RA conjugated miR-29b mimics (Mimic #4, Mimic #6 and Mimic #7) were evaluated in an acute mouse CCl₄-induced mouse model of liver fibrosis. Mice were treated with either CCl₄ or oil at 1 ml/kg by intraperitoneal (IP) injection twice a week for two weeks. The miR-29b mimics (at a dosage of 10 mg/kg) or an equal volume of saline (control) were administered by intravenous (IV) injection three times a week for two weeks (FIG. 12A). After takedown at day 16, liver tissues were analyzed for pharmacodymanics (target engagement/activity/efficacy). RT-PCR for a panel of genes known to be regulated by miR-29b showed the down regulation by all three mimics (FIG. 12B).

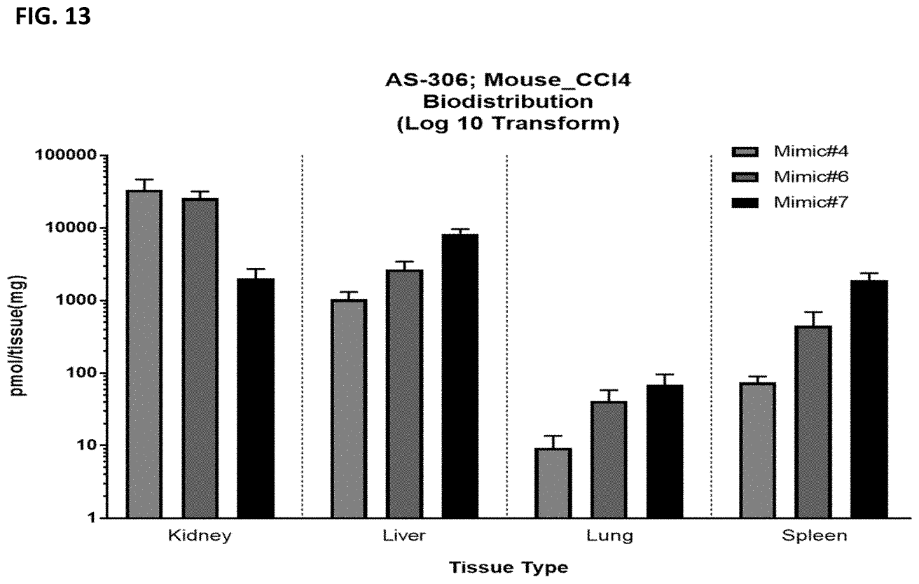

Biodistribution of the compounds in this animal model was also determined using a hybridization based assay (FIG. 13). Interestingly, the three compounds demonstrated different tissue specific accumulation profiles. Mimic #7 was present in the highest levels in the liver, lung and spleen, followed by Mimic #6 and Mimic #4. In contrast the relative levels of compounds in the kidney followed the order Mimic #4>Mimic #6>Mimic #7.

### Example 6—Synthesis and In-Vivo Evaluation of miR-29b Mimic Conjugated to Docosanoic Acid (DCA)

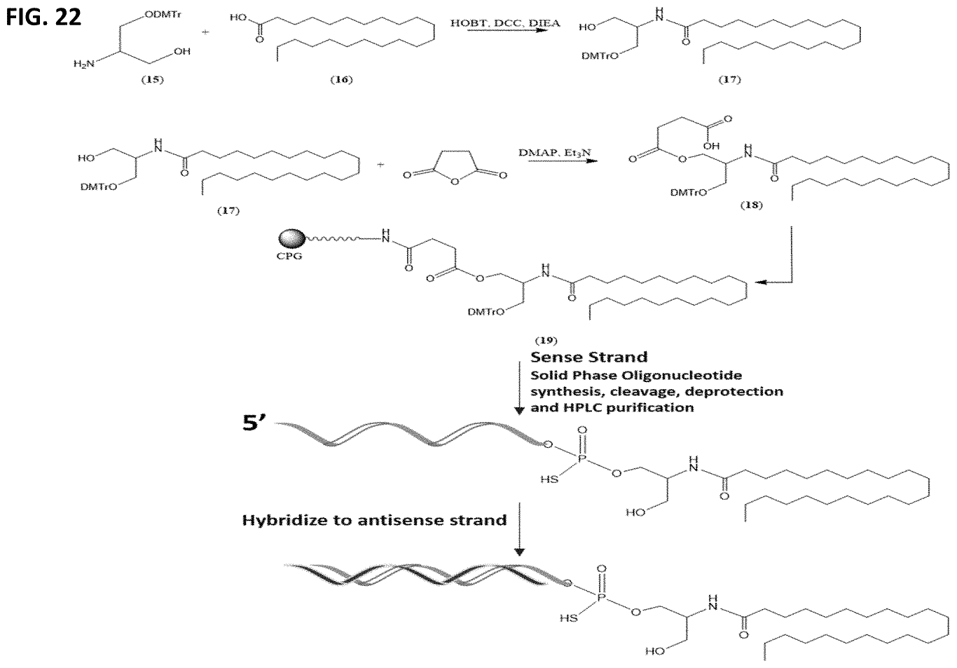

Controlled pore glass (CPG) solid support loaded with docosanoic acid (19) was prepared as described in FIG. 1. Subsequently, the sense strand was synthesized using 19 as the solid support and utilizing standard solid phase oligonucleotide synthesis protocols. See FIG. 22. After cleavage from the solid support, deprotection and purification, the strand was hybridized to the antisense strand to produce the miR-29b mimic. Alternately, 18 can be converted to the corresponding amidite and incorporated into 5′ end of the sense as the last coupling during the solid phase synthesis process. Similar to above, after cleavage, deprotection and purification, hybridization to the antisense strand can produce the desired double stranded microRNA mimic.

Studies will be carried out in a bleomycin-treated mouse model of IPF, a liver fibrosis mouse model and a kidney fibrosis mouse model to evaluate the delivery of the DCA-conjugated miR-29b mimics to fibrotic tissues, and measure the subsequent down-regulation of miR-29b target genes, per the experimental methods described in Examples 3-5.

### Example 7—Synthesis and In Vivo Evaluation of Bis-Monocyclic Peptide-Conjugated miR29-Mimics

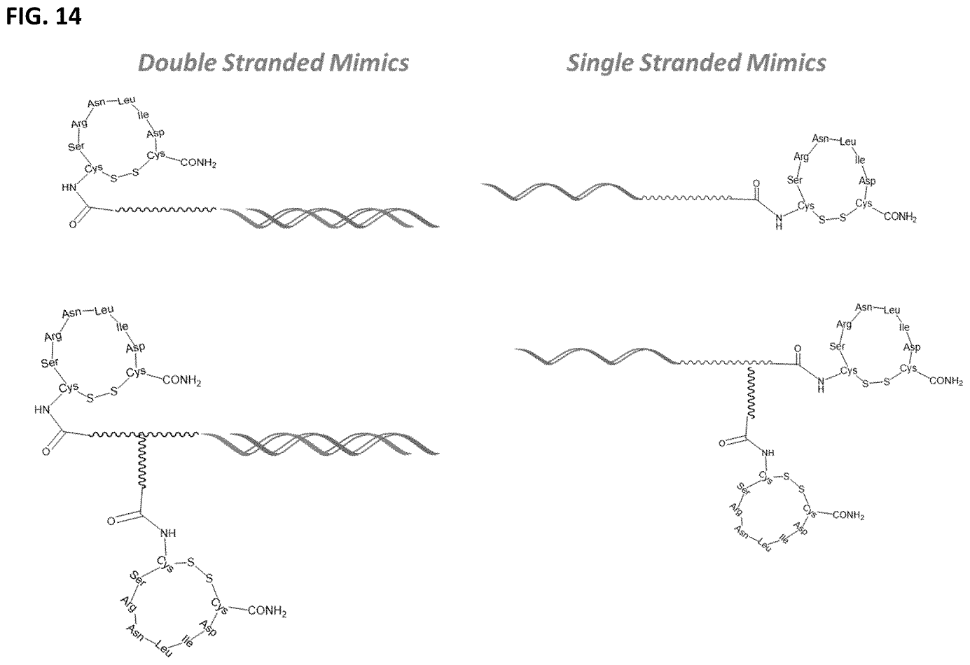

PDGFβ-R targeting peptide-miR-29b mimic conjugates having alternate configurations shown in FIG. 14 will be generated. Further, bicyclic peptide derived from the human PDGF-β protein sequence will also be conjugated to the miR-29b mimics disclosed here. Additionally, miR-29b mimics conjugated with bis-monocyclic peptides will be generated as described below.

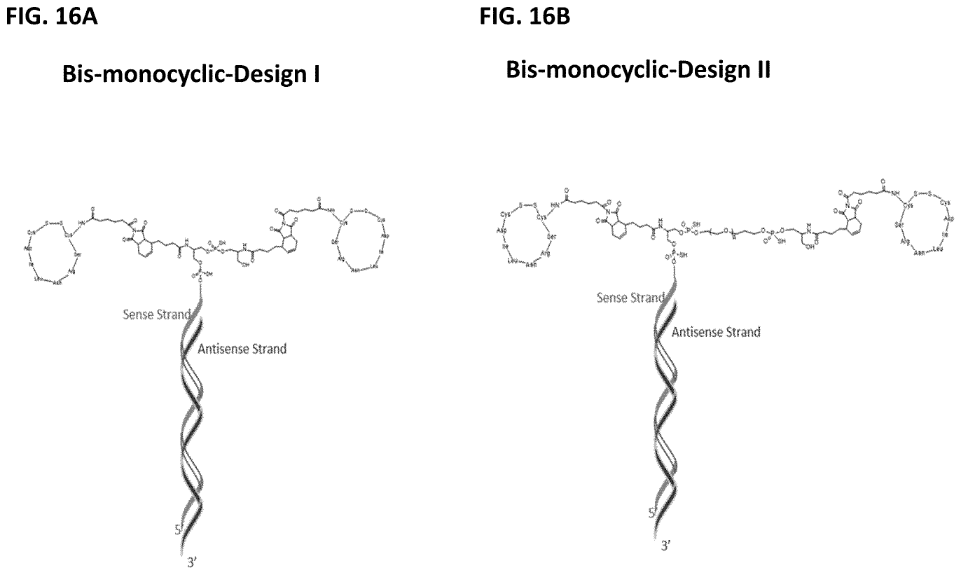

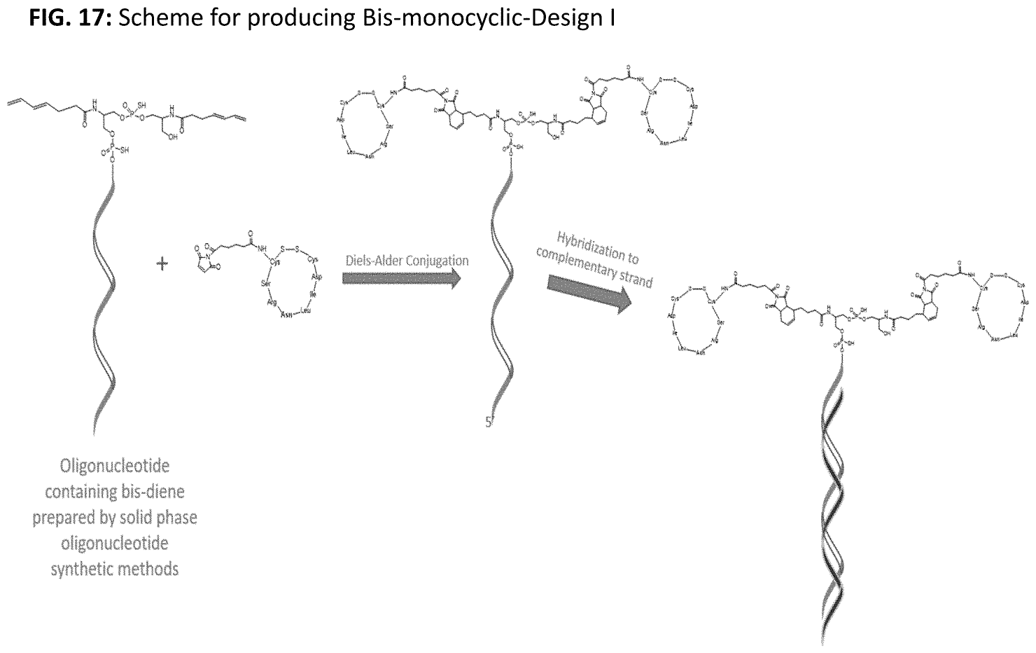

Structural designs of bis-monocylic peptides are shown in FIG. 16. A sense strand of miR-29b mimic having bis-diene on the 3′ end will be prepared by solid phase polynucleotide synthetic methods and then, conjugated to monocyclic peptides by Diels-Alder conjugation, as shown in FIG. 17. In the next step, the bis-monocyclic peptide-conjugated sense strand of miR-29b mimic will be hybridized with its complementary strand to arrive at a double stranded miR-29b mimic conjugated to a bis-monocyclic peptide, as shown in FIG. 16A.

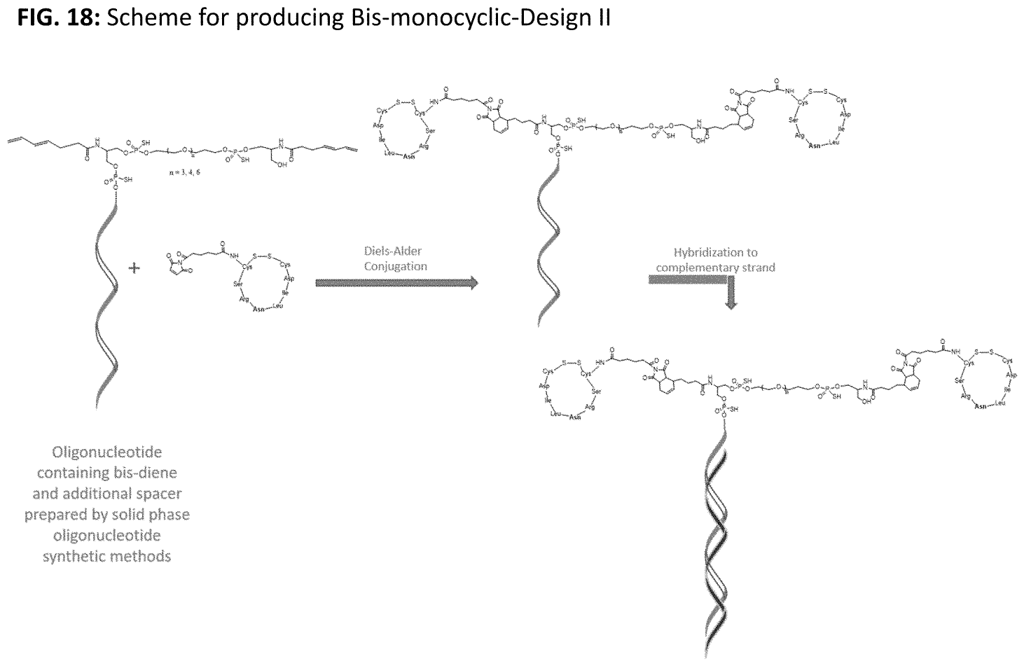

In another method, sense strand of miR-29b mimic containing bis-diene and an additional spacer on the 3′ end will be prepared by solid phase polynucleotide synthetic methods and then, conjugated by Diels-Alder conjugation to a monocyclic peptide, as shown in FIG. 18. The bis-monocyclic peptide-conjugated polynucleotides will then be hybridized with its complementary strand to arrive at a double stranded miR-29b mimic conjugated to a bis-monocyclic peptide, as shown in FIG. 16B.

Studies will be carried out in a bleomycin-treated mouse model of IPF, a liver fibrosis mouse model and a kidney fibrosis mouse model to evaluate the delivery of all the conjugated miR-29b mimics described above to fibrotic tissues, and measure the subsequent down-regulation of miR-29b target genes, per the experimental methods described in Examples 3-5.

### Example 8—In Vitro and In Vivo Evaluation of Human PDGF BiPPB Conjugated miR-29 Mimics

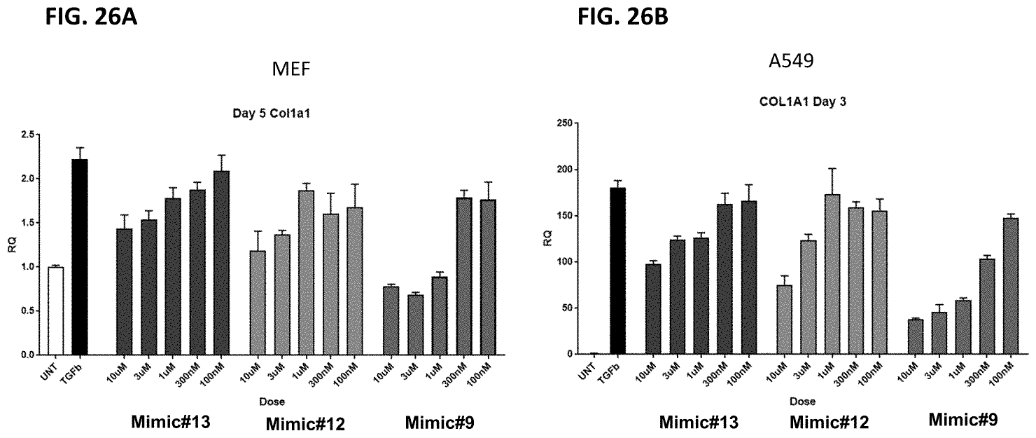

An in vitro study was carried out to assess the efficacy of human PDGF BiPPB conjugated miR-29 mimics and mouse PDGF BiPPB conjugated miR-29 mimics. Two different cell lines, mouse embryonic fibroblasts (MEFs) and a human lung epithelial cell line (A549), were used in the in vitro study (FIGS. 26A-26B). Treatment of either cell-line with TGFb induced Col1a1 expression. Three miR-29 mimics were used in this study: a human PDGF BiPPB conjugated miR-29 mimic (Mimic #12), a mouse PDGF BiPPB conjugated miR-29 mimic (Mimic #13), and a cholesterol-conjugated miR-29 control compound (Mimic #9). Upon application of any of these miR-29 mimics, both cell lines displayed a dose-dependent decrease in Col1a1 expression, demonstrating that both mouse and human PDGF BiPPB conjugated miR-29 mimics are effective for down-regulating collagen related genes.

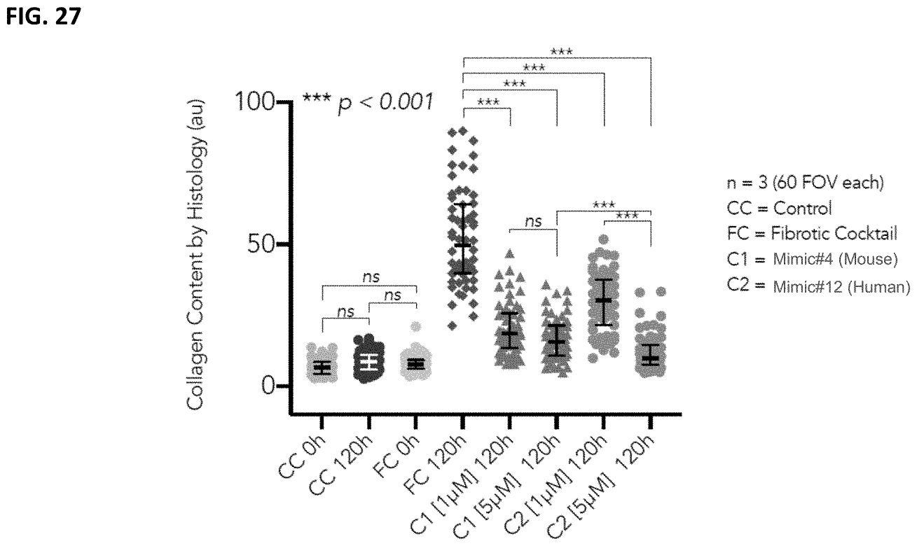

To further assess the functional effects of these BiPPB conjugated miR-29 mimics, an ex vivo study was performed using human precision-cut lung slices (FIG. 27). Human lung tissue was treated with a control cocktail (CC) or fibrotic cocktail (FC). The FC comprises 5 ug TGFb, 50 ug PDGF-AB, 10 ng TNFa, and 10 mg LPA which can induce the fibrotic response. Upon application of either mouse or human PDGF BiPPB conjugated miR-29 mimic, the human lung tissue samples displayed significant down-regulation of collagen as assessed by quantitative imaging of collagen.

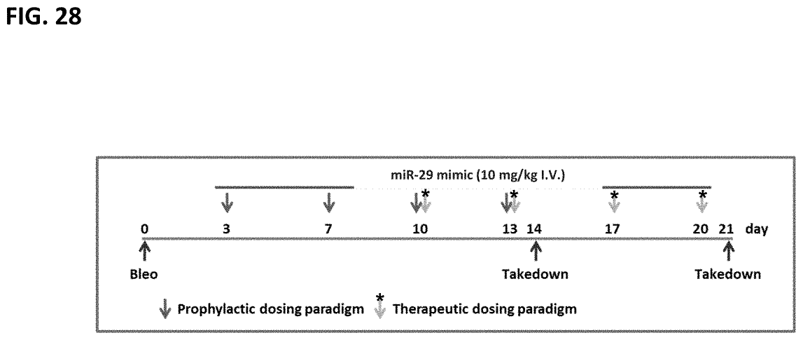

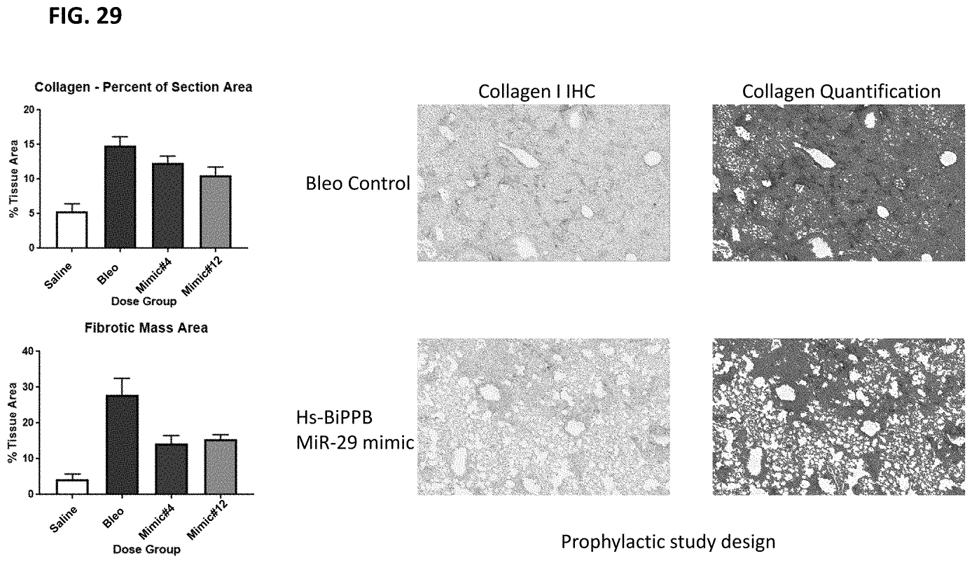

To further determine the efficacy of these peptide conjugated miR-29 mimics, an in vivo study was performed using bleomycin-induced fibrotic mice (FIGS. 28-29). As shown in FIG. 28, mice were given bleomycin at day 0 to induce the fibrotic response. At days 3, 7, 10, and 13, mice were treated intravenously with either a mouse PDGF BiPPB conjugated miR-29 mimic (Mimic #4) or a human PDGF BiPPB conjugated miR-29 mimic (Mimic #12) at 10 mg/kg dosage. Tissue was harvested at day 14 and collagen immunohistochemistry was performed on the lungs to assess fibrosis and to evaluate collagen deposition (FIG. 29). Application of either mouse or human PDGF BiPPB conjugated miR-29 mimic blunts the collagen deposition following bleomycin treatment, demonstrating their efficacy in vivo.

1. A polynucleotide conjugate comprising a polynucleotide and an agent, wherein the polynucleotide comprises a double stranded miR-29 mimic, each strand comprising a plurality of nucleotide modifications, and wherein the agent facilitates the delivery of the polynucleotide to a cell type or tissue type involved in fibrosis or inflammation.

2. A polynucleotide conjugate comprising a polynucleotide and an agent, wherein the polynucleotide comprises a double stranded miR-29 mimic, each strand comprising a plurality of nucleotide modifications, and wherein the agent comprises a fibrotic pathway agent.

3. The polynucleotide conjugate of claim 1, wherein the agent comprises a fibrotic pathway agent.

4. The polynucleotide conjugate of claim 1, wherein the agent is selected from a peptide, a small molecule, and a fatty acid.

5. The polynucleotide conjugate of claim 3, wherein the fibrotic pathway agent is a fibrotic pathway targeting agent.

6. The polynucleotide conjugate of claim 3, wherein the fibrotic pathway agent is a fibrotic pathway activating agent.

7. The polynucleotide conjugate of claim 3, wherein the fibrotic pathway agent is a fibrotic pathway inhibiting agent.

8. The polynucleotide conjugate of claim 4, wherein the agent is a peptide.

9. The polynucleotide conjugate of claim 8, wherein the peptide is a cyclic peptide.

10. The polynucleotide conjugate of claim 8, wherein the peptide comprises at least one copy of the amino acid sequence SRZLID, wherein Z is either an asparagine (N) or an arginine (R).

11. The polynucleotide conjugate of claim 9, wherein the cyclic peptide is a bicyclic peptide

12. The polynucleotide conjugate of claim 9, wherein the cyclic peptide comprises the amino acid sequence X1SRZLIDX2-linker-X3SRZLIDX4, wherein Z is either an asparagine (N) or an arginine (R), wherein the pair of X1 and X2, and the pair of X3 and X4 can form a peptidic bond to form a bicyclic structure, and wherein the sequences SRZLID are each part of a ring, and optionally wherein the linker is of size n, wherein n is 1-20.

13. The polynucleotide conjugate of claim 8, wherein the peptide is a receptor-binding peptide.

14. The polynucleotide conjugate of claim 13, wherein the peptide is a PDGF receptor-binding peptide.

15. The polynucleotide conjugate of claim 14, wherein the PDGF receptor-binding peptide is derived from PDGF-beta.

16. The polynucleotide conjugate of claim 4, wherein the fibrotic pathway agent is a small molecule.

17. The polynucleotide conjugate of claim 16, wherein the small molecule is a retinoid.

18. The polynucleotide conjugate of claim 17, wherein the retinoid is an all trans retinoid.

19. The polynucleotide conjugate of claim 17, wherein the retinoid is retinoic acid.

20. The polynucleotide conjugate of claim 4, wherein the fibrotic pathway agent is a fatty acid.

21. The polynucleotide conjugate of claim 20, wherein the fatty acid is a long chain fatty acid.

22. The polynucleotide conjugate of claim 21, wherein the long fatty acid comprises 8-30 carbons.

23. The polynucleotide conjugate of claim 21, wherein the long fatty acid is a long-chain dicarboxylic acid.

24. The polynucleotide conjugate of claim 21, wherein the long fatty acid is docosanoic acid.

25. The polynucleotide conjugate of claim 1, wherein the miR29 mimic comprises a first strand comprising a mature miR-29a, miR-29b, or miR-29c sequence, and a second strand comprising a sequence that is substantially complementary to the first strand.

26. The polynucleotide conjugate of claim 25, wherein the first strand comprises a mature miR-29a.

27. The polynucleotide conjugate of claim 25, wherein the first strand comprises a mature miR-29b.

28. The polynucleotide conjugate of claim 25, wherein the first strand comprises a mature miR-29c.

29. The polynucleotide conjugate of claim 25, wherein the agent is conjugated to the first strand.

30. The polynucleotide conjugate of claim 29, wherein the agent is conjugated to the 5′-end of the first strand.

31. The polynucleotide conjugate of claim 29, wherein the agent is conjugated to the 3′-end of the first strand.

32. The polynucleotide conjugate of claim 25, wherein the agent is conjugated to the second strand.

33. The polynucleotide conjugate of claim 32, wherein the agent is conjugated to the 5′-end of the second strand.

34. The polynucleotide conjugate of claim 32, wherein the agent is conjugated to the 3′-end of the second strand.

35. The polynucleotide conjugate of claim 28, wherein the agent is conjugated to an internal position of the second strand.

36. The polynucleotide conjugate of claim 1, wherein the agent is conjugated to the miR-29 mimic via a linker.

37. The polynucleotide conjugate of claim 1, wherein the agent is conjugated to the miR-29 mimic via cholesterol.

38. The polynucleotide conjugate of claim 1, wherein each nucleotide of the first strand is modified.

39. The polynucleotide conjugate of claim 1, wherein each nucleotide of the second strand is modified.

40. The polynucleotide conjugate of claim 1, wherein at least 50% of the nucleotides in the first strand are modified.

41. The polynucleotide conjugate of claim 1, wherein at least 50% of the nucleotides in the second strand are modified.

42. The polynucleotide conjugate of claim 1, wherein the nucleotides of at least 50% of the first and at least 50% of the second strand are modified.

43. The polynucleotide conjugate of claim 1, wherein each nucleotide of the first strand, and each nucleotide of the second strand is modified.

44. The polynucleotide conjugate of claim 1, wherein the first strand comprises a polynucleotide sequence selected from SEQ ID Nos. 6-9, 23-32, 38-41, 55-65 and 74-77.

45. The polynucleotide conjugate of claim 1, wherein the second strand comprises a polynucleotide sequence selected from SEQ ID Nos. 1-5, 10-22, 33-37, 42-54, 66-73, and 97-98.

46. The polynucleotide conjugate of claim 1, wherein the first strand comprises a polynucleotide sequence selected from SEQ ID Nos. 74-77 and 55.

47. The polynucleotide conjugate of claim 1, wherein the second strand comprises a polynucleotide sequence selected from SEQ ID Nos. 66-73, 97, 98, 45, 53 and 54.

48. The polynucleotide conjugate of claim 1, wherein the second strand comprises a polynucleotide sequence of SEQ ID NO: 68, and the first strand comprises a polynucleotide sequence of SEQ ID NO: 75.

49. The polynucleotide conjugate of claim 1, wherein the second strand comprises a polynucleotide sequence of SEQ ID NO: 69, and the first strand comprises a polynucleotide sequence of SEQ ID NO: 55.

50. The polynucleotide conjugate of claim 1, wherein the second strand comprises a polynucleotide sequence of SEQ ID NO: 71, and the first strand comprises a polynucleotide sequence of SEQ ID NO: 55.

51. The polynucleotide conjugate of claim 1, wherein the second strand comprises a polynucleotide sequence of SEQ ID NO: 72, and the first strand comprises a polynucleotide sequence of SEQ ID NO: 55.

52. The polynucleotide conjugate of claim 1, wherein the second strand comprises a polynucleotide sequence of SEQ ID NO: 97, and the first strand comprises a polynucleotide sequence of SEQ ID NO: 55.

53. The polynucleotide conjugate of claim 1, wherein the second strand comprises a polynucleotide sequence of SEQ ID NO: 98, and the first strand comprises a polynucleotide sequence of SEQ ID NO: 55.

54. The polynucleotide conjugate of claim 1, wherein the second strand comprises a polynucleotide sequence of SEQ ID NO: 53, and the first strand comprises a polynucleotide sequence of SEQ ID NO: 55.

55. The polynucleotide conjugate of claim 1, wherein the second strand comprises a polynucleotide sequence of SEQ ID NO: 42, and the first strand comprises a polynucleotide sequence of SEQ ID NO: 60.

56. The polynucleotide conjugate of claim 1, wherein the second strand comprises a polynucleotide sequence of SEQ ID NO: 43, and the first strand comprises a polynucleotide sequence of SEQ ID NO: 55.

57. The polynucleotide conjugate of claim 1, wherein the second strand comprises a polynucleotide sequence of SEQ ID NO: 44, and the first strand comprises a polynucleotide sequence of SEQ ID NO: 60.

58. The polynucleotide conjugate of claim 1, wherein the second strand comprises a polynucleotide sequence of SEQ ID NO: 45, and the first strand comprises a polynucleotide sequence selected from SEQ ID NOs: 55-57.

59. The polynucleotide conjugate of claim 1, wherein the second strand comprises a polynucleotide sequence of SEQ ID NO: 46, and the first strand comprises a polynucleotide sequence of SEQ ID NO: 55.

60. The polynucleotide conjugate of claim 1, wherein the second strand comprises a polynucleotide sequence of SEQ ID NO: 47, and the first strand comprises a polynucleotide sequence of SEQ ID NO: 61.

61. The polynucleotide conjugate of claim 1, wherein the second strand comprises a polynucleotide sequence of SEQ ID NO: 48, and the first strand comprises a polynucleotide sequence of SEQ ID NO: 62.

62. A pharmaceutical composition comprising the polynucleotide conjugates of claim 1.

63. A method of treating fibrosis or a fibrosis-related condition comprising administering to a subject in need a therapeutically effective amount of: a polynucleotide conjugate comprising a polynucleotide and an agent, wherein the polynucleotide comprises a double stranded miR-29 mimic, each strand comprising a plurality of nucleotide modifications, and wherein the agent facilitates the delivery of the polynucleotide to a cell type or tissue type involved in fibrosis or inflammation, or
a pharmaceutical composition comprising a polynucleotide conjugate comprising a polynucleotide and an agent, wherein the polynucleotide comprises a double stranded miR-29 mimic, each strand comprising a plurality of nucleotide modifications, and wherein the agent facilitates the delivery of the polynucleotide to a cell type or tissue type involved in fibrosis or inflammation.
